(19) United States
(12) Patent Application Publication
(10) Pub. No.: US 2024/0263161 A1
(43) Pub. Date: Aug. 8, 2024

(54) PEPTIDES WITH MUCIN-BINDING PROPERTIES

(71) Applicant: University of Copenhagen, København (DK)

(72) Inventors: Yoshiki Narimatsu, København (DK); Christian BÜLL; Rebecca NASON, København (DK); Bernard Henrissat, København (DK); Henrik Clausen, København (DK)

(21) Appl. No.: 18/566,290

(22) PCT Filed: Jun. 3, 2022
(86) PCT No.: PCT/EP2022/065169

§ 371 Date: Dec. 1, 2023

(30) Foreign Application Priority Data
Jun. 4, 2021 (EP) 21177857.6

(51) Int. Cl. C12N 9/52; A61K 38/00; C07K 14/195

(52) U.S. Cl. CPC C12N 9/52; C07K 14/195; C12Y 304/24; A61K 38/00; C07K 2319/33

(57) ABSTRACT

There are provided compositions and mucin-binding targeting agents derived from microbial proteins that have selective binding properties for densely glycosylated mucins as well as such compositions and targeting agents comprising a binding moiety and/or a pay load which may be attached to the binding moiety or directly to a targeting agent. The compositions and targeting agents may be used as medicaments in the treatment of a disease, illness, or disorders.

### FIELD OF THE INVENTION

The present invention relates to mucin-binding targeting agents comprising peptides derived from microbial proteins that have selective binding properties for densely glycosylated mucins. Furthermore, the invention relates to use of such targeting agents to bind mucin layers covering mucosal surfaces such as gastric and colonic mucous and mucosal lining epithelia. The provided mucin-binding agents are useful for detecting and binding mucins and mucosal surfaces for biomarker and therapeutic purposes. The invention also relates to methods for the use of mucin-binding agents for targeting and delivery of therapeutic agents to mucosal surfaces.

### BACKGROUND OF THE INVENTION

### Mucins

Mucins are a large family of heavily glycosylated proteins that line all mucosal surfaces and represent the major macromolecules in body fluids¹. Mucins clear, contain, feed, direct, and continuously replenish our microbiomes, limiting unwanted co-habitation and repressing harmful pathogenic microorganisms². Mucins in the gut constitute the primary barrier as well as the ecological niche for the microbiome. Dynamic replenishment of mucin layers provides constant selection of the resident microbiome through adhesive interactions, and degradation of mucin O-glycans by members of the microbiota supply nutrients^{3,4,2,5}. Mucin O-glycans present the essential binding opportunities and informational cues for microorganisms via adhesins, however, our understanding of these features is essentially limited to results from studies with simple oligosaccharides without the protein context of mucins and the higher order features presented by dense O-glycan motifs. Mucins are notoriously difficult to isolate due to their size and heterogeneity, and production by recombinant expression in cell lines is impeded due to difficulties with the assembly of full coding expression constructs often resulting in heterogeneous products⁶.There are at least 18 distinct mucin genes encoding membrane or secreted mucins in the human genome⁷. The large gel-forming secreted mucins may form oligomeric networks or extended bundles through inter- and intramolecular disulfide bridges in the C- and N-terminal cysteine-rich regions¹. A common characteristic of all mucins is that the major part of their extracellular region is comprised of variable number of imperfect tandem repeated (TR) sequences that carry dense O-glycans, with the notable exception of MUC16 that contains a large, densely O-glycosylated N-terminal region without TRs^{8,9}. These TR regions appear poorly conserved throughout evolution in contrast to the flanking regions of the large mucins¹⁰, and this is generally interpreted to reflect that the TR regions simply need to carry dense O-glycans without specific patterns or functional consequences.

Mucins line all mucosal surfaces and represent one of the most abundant components of body fluids including saliva^{5,11}. Gel or polymer-forming secreted mucins (MUC2, MUC5AC, MUC5B, and MUC6) cover the gastrointestinal tract. In the stomach a net-like layer of MUC5AC polymers forms a diffusion barrier protecting the surface lining epithelium¹². In the small intestine a non-attached mucus layer exists, mainly comprised of MUC2. The large intestine, however, is covered by a thick two-layered MUC2 network with a dense inner layer that serves as a molecular sieve impenetrable by e.g. bacteria, and an outer loose MUC2 network that contains the microbiome at a distance from the surface epithelium. These mucin layers cover and protect the lining mucosa and present an obstacle for delivery of therapeutics through mucosal surfaces¹³.

Mucins arguably represent a last frontier in analytics of glycoproteins. Most mucins are extremely large and heterogenous glycoproteins that are resistant to conventional glycoproteomics strategies that are dependent on proteolytic fragmentation and sequencing¹⁴⁻¹⁶, and despite increasing knowledge of O-glycosites⁹, identification of actual sites of glycosylation in mucins is essentially limited to MUC1^{17,18,19}, lubricin²⁰, and the large N-terminal mucin-like region of MUC16⁹. Current understanding of mucins, their glycosylation, and their functions is therefore still highly limited.

### Glycomucinases—the Glycoprotease StcE

Secreted protease of C1 esterase inhibitor (StcE) from Enterohemorrhagic Escherichia coli (EHEC) O157:H7 is a zinc metalloprotease with remarkable ability for cleaving densely O-glycosylated mucins and mucin-like glycoproteins. StcE is thought to serve in colonic mucin degradation facilitating EHEC adherence to the epithelium and subsequent infection^{21,22}. Recent studies demonstrated that StcE has selective substrate specificity for S/T-X-S/T motifs with a requirement for O-glycans at the first S/T residue²³. The StcE glycoprotease was demonstrated to cleave a wide array of isolated mucins or cell-membrane mucins endogenously expressed by cancer cell lines, including cell surface mucins, such as MUC1 and MUC16, and mucin-like O-glycoproteins such as CD43 and CD45, revealing its broad substrate specificity with mucins and mucin-like glycoproteins²³.

Moreover, a catalytically inactive mutant of StcE (StcE^{E447D}) has glycan-binding properties for the dST core1 O-glycan as evaluated by printed glycan arrays²⁴, but also exhibit general binding properties for mucins^{23,25-27}. It has been proposed that StcE plays a role in adherence of EHEC to the intestinal epithelium by binding to mucins^{24,25,28}. More recently, Bertozzi and colleagues took advantage of a classical strategy to employ the catalytic unit of an enzyme for use as a binding molecule²⁹. This involves inactivating the catalytic function of the enzyme and retaining the substrate binding properties of the catalytic unit of the enzyme. Thus, the catalytically inactive mutant of StcE (StcE^{E447D}) was used for binding studies with tissue sections and binding to mucin secreting cells by use of histological tissue sections was demonstrated²⁷ [PCT/US2019/060346]. StcE has also been suggested to bind O-glycans on a glycan array and in particular to the core1 disialyl-T (dST) O-glycan²⁷. Moreover, a recent report described use of the StcE^{E447D} mutant for affinity isolation of O-glycoproteins demonstrating that the enzyme can be used to enrich mucins and a variety of other O-glycoproteins many of which are not mucins or mucin-like such as LRP8³⁰.

### Cell-based Glycan Arrays

State-of-the-art technologies to capture the informational content of mucins are confined to studies with synthetic and isolated O-glycans³¹, synthetic and chemoenzymatically produced short glycopeptides³², and synthetic glycopeptides as well as non-natural polymers^{33,34}; all of which are rare commodities that do not reflect the complex information captured in distinct human mucins by their display of patterns and structures of O-glycans. With the advent of the facile nuclease-based gene engineering technologies it has become possible to engineer mammalian cells with combinatorial knockout (KO) and knockin (KI) of glycosylation genes to display subsets and distinct features of the glycome on the cell surface or on secreted reporter proteins in order to probe biological interactions dependent on glycans³⁵⁻³⁷ [US patent applications US20210087587 and US20190330601]. The genetic engineering provides opportunities for interpretation and dissection of the glycosylation genes, biosynthetic pathways, and structural features required for identifiable interactions with the cell library³⁸. The cell-based glycan array is a sustainable platform to display the human glycome and enabling wide surveying of the informational content of human glycans. Use of the cell-based glycan display is fundamentally different from printed glycan arrays in that the primary read-out is consequences of loss/gain of glycosyltransferase genes that are used to predict structural glycan features. Printed glycan arrays provide direct information of glycan haptens involved in interactions, while the cell-based array provides comprehensive knowledge of genes regulating the expression of glycan features.

The cell-based glycan array strategy was previously used to express designed protein reporter constructs containing the high density O-glycan regions (O-glycodomains) derived from the stem region of GP1bα and TRs from several different mucins³⁶. The reporters were transiently expressed in HEK293^{WT} cells and used to demonstrate that bacterial adhesins from Streptococcus show differential binding to cells displaying different mucin reporters. These studies suggested that the adhesins recognize specific O-glycan structures in ways influenced by their presentation on the protein or mucin backbone. Analysis of the sequences used for the reporter constructs derived from the mucin TRs and GP1bα did not reveal simple common sequence motifs shared among those providing binding for the Siglec-like adhesins, and thus, the data could not be used to define the recognition motifs in further detail. Recognition of “clustered saccharide patches” orchestrated by positions, spacing and direct interactions of multiple glycans in a protein was earlier proposed to provide expanded binding specificities and high affinity interactions, and evidence in support of this has been found with all types of glycoconjugates^{39,40}.

Printed glycan arrays have transformed the field of glycosciences and served as essential tools for exploring the interactome of glycans and proteins⁴¹; however, studies have also resulted in the emergence of an interesting conundrum in explaining diversity of pathogen interactions and their host tropism in nature^{39,42}. Results from printed glycan arrays indicate that relatively few distinct glycan motifs serve as common ligands for many microbial adhesins and glycan-binding proteins⁴². The core structural motifs recognized, typically only 3-5 monosaccharides, are even more limited since glycans are built on common scaffolds with units such as N-Acetyllactosamine (LacNAc)^{42,43} Many host-pathogen interactions identified involve terminal sialic acid residues on common structural motifs, and while the glycosidic linkages, the underlying core structures, and numerous modifications of sialic acids⁴⁴, do vary to a degree, the overall structural permutations are still somewhat limited.

Current knowledge of the molecular basis for host-pathogen interactions are therefore incomplete, and essentially limited to binding and recognition of different simple oligosaccharide structures by lectins, adhesins, agglutinins and other carbohydrate-binding modules^{42,43}. Such oligosaccharides may be widely found on glycolipids, glycoproteins including mucins, proteoglycans, other types of glycoconjugates, and as free oligosaccharides found widely on cells and in body fluids throughout the body⁴⁵. Binding to such glycan epitopes by microbial glycan-binding molecules therefore have limited cell-type and organ specificity.

### SUMMARY OF THE INVENTION

The present invention relates to microbial peptide modules, that bind to select human and/or other mammalian mucins and do not bind to simple oligosaccharides. Microbial peptide modules of the invention bind to the tandem repeat regions of human mucins when these have clusters of O-glycans attached, as found on mucins in normal and diseased mucosa. The present invention also relates to methods of use of such microbial peptide modules in mucin-binding targeting agents for binding to mucins found in the mucous layers of human lining epithelia. Moreover, the present invention provides the use of such binding modules to detect mucins and mucous layers, and use of these modules to deliver pharmacological agents to mucosal surfaces.

The mucin-binding targeting agents present several advantages, including high selectivity for preferred mucins, such as MUC5Ac, and preference for binding non-truncated sugars over truncated sugars. Moreover, the mucin-binding targeting agents bind selected mucins with high affinity, such as in the nanomolar range.

The nanomolar range binding affinities of the mucin-binding targeting agents are improved markedly compared to other lectin-based systems (micromolar) and on par with binding affinities achieved by antibodies. Moreover, the high selectivity ensures that any attached payload can be delivered with high precision to the intended tissue expressing the targeted mucin(s), while the preference for non-truncated sugars limits off-target binding to truncated sugars, e.g. STn and Tn, which are associated with shed degraded mucins from the mucus.

### BRIEF DESCRIPTION OF THE DRAWINGS

FIG. 1 Illustrates design of the human mucin tandem repeat (TR) display platform. Illustration of the mucin TR display approach with membrane bound and secreted mucin reporters expressed in KO/KI glycoengineered isogenic HEK293 cell lines. HEK293 wild type (WT) cells are predicted to produce a mixture of mSTa, dST and sialylated core2 structures, and through stable genetic engineering a library of isogenic HEK293 cells with different O-glycosylation capacities were developed. These cells enable display of mucin TRs with different O-glycan structures as indicated (glycan symbols and genetic design shown) as well as tunable site occupancy by engineering of the GALNT isoenzyme gene repertoire (left part). The secreted mucin reporter construct design contains an N-terminal 6xHis and FLAG tag and GFP followed by different mucin TR domains of ca. 200 amino acids (single TR domains used for MUC3, MUC5B, MUC13, MUC6 and GP1bα) and a second C-terminal 6xHis tag. The membrane bound mucin reporter constructs contains a N-terminal FLAG tag and GFP followed by the mucin TR domain and further includes the SEA and transmembrane domain of human MUC1 in the C-terminal for membrane retention. The most characteristic TR sequence for each construct is illustrated with the number of TRs included (right part). Full sequences and glycosylation patterns of the TRs are shown in FIG. 2 and recited in Table 1. Transient or stable expression of the mucin TR reporters in the glycoengineered isogenic HEK293 cell library enables display of cell surface mucin TRs as well as production of secreted mucin TRs with distinct O-glycan structures. Structures of glycans are shown with symbols drawn according to the Symbol Nomenclature for Glycans (SNFG) format^{46.} Note that colors are omitted, but illustrated HexNAc, Hex, and sialic acid represent GalNAc, Gal and NeuAc.

FIG. 2 represents a schematic presentation of the imperfect TR amino acid sequences selected for design of the human mucin TR reporters. All Ser/Thr residues are highlighted as potential O-glycosites by glycan symbols (mSTa O-glycans shown for simplicity) to illustrate the characteristic patterning generated with all Ser/Thr residues O-glycosylated. The representation is to highlight the spacing of glycans on the TR sequence, and the actual sequences used for each of the human mucin TR constructs shown are found in Table 1.

FIG. 3 illustrates analysis of O-glycosylation of the mucin TR reporters expressed in glycoengineered HEK293 cells with antibodies and lectins. Panel a Flow cytometry analysis of binding of lectins and anti-carbohydrate mAbs to engineered HEK293 cells transiently expressing membrane-bound mucin TR reporters (GFP and FLAG-tagged) as indicated. Primary specificities illustrated with glycan symbols. GFP negative cells (non-transfected) or GFP positive cells (transfected) were analyzed by flow cytometry and mean fluorescent intensity (MFI) values presented as a heat map. Surface expression of mucin TR reporters was confirmed by anti-FLAG antibody labelling. Panel b Flow cytometry analysis of binding of mucin-specific mAbs to HEK293WT and HEK293KO C1GALT1 cells transiently expressing mucin TR reporters. Panel c Flow cytometry analysis of binding of MUC1 glycoform-specific mAbs to glycoengineered HEK293 cells stably expressing the MUC1 TR reporter. MFI values from representative experiments are shown (greytones indicate high to low MFI values).

FIG. 4 Illustrates SDS-PAGE Coomassie analysis of purified secreted mucin TR reporters. Panel a Analysis of TR reporters expressed in HEK293WT with heterogeneous core1/2 O-glycans. Panel b Analysis of TR reporters expressed in HEK293KO C1GALT1 with homogenous Tn O-glycans.

FIG. 5 Shows ELISA analysis of purified secreted MUC1 and MUC7 TR reporters produced in glycoengineered HEK293 cells. Panel a SDS-PAGE analysis of MUC1 TR reporters (left) and corresponding ELISA antigen titrations with lectins and anti-carbohydrate mAbs (right) as indicated. Anti-Flag mAb was included to evaluate comparable coating efficiencies. Panel b The same analysis with MUC7 TR reporters. Samples loaded for SDS-PAGE analysis corresponded to symbol key (top-to low illustrated right) as follows: Lane 1 HEK293^{WT}, lane 2 HEK293^{KO GCNT1}, lane HEK293^{KO GCNT1/ST3GAL1/2}, lane 4 HEK293^{KO GCNT1/ST3GAL1/2 ST6GALNAC2,3,4}, lane 5 HEK293^{KO C1GALT1}, lane 6 HEK293^{KO COSMC KI ST6GALNAC}, lane 7 HEK293^{KO COSMC KI B3GNT6}. Structures of glycans are shown with symbols drawn according to the Symbol Nomenclature for Glycans (SNFG) format⁴⁴. Note that colors are omitted, but illustrated HexNAc (open/grey), Hex, and sialic acid represent GalNAc/GlcNAc, Gal and NeuAc.

FIG. 6 Illustrates HPLC isolation of the O-glycodomains from secreted MUC1 TR reporters for intact mass analysis. Panel a C4 HPLC isolation of the undigested Tn-glycosylated MUC1 TR reporter with GFP expressed in HEK293^{KO CIGALT1} cells. Panel b C8 HPLC separation of the corresponding LysC digested TR reporter with the O-glycodomain eluting in fractions 22-23 and the intact GFP module in fraction 33 as verified by VVA lectin ELISA. Panel c C4-HPLC of LysC digested T-MUC1 with the O-glycodomain eluting in fractions 22-26 and the intact GFP module in fraction 35 as verified by PNA lectin ELISA, and Panel d further digested by AspN. The intact GFP-tagged reporter eluted at ˜60% acetonitrile, while the released TR O-glycodomains eluted at ˜35% and the digested GFP-tag at ˜55%.

FIG. 7 Illustrates mass spectrometry analysis of secreted MUC1 TR reporter O-glycoforms. Panel a Deconvoluted intact mass spectra of secreted, purified the MUC1 reporter produced in HEK293^{KO C1GALT1}, HEK293^{KI ST6GALNAC1}, HEK293^{KO GCNT1, ST3GAL1/2} and HEK293^{KO GCNT1} cells. Reporters were treated with neuraminidase to remove sialic acids and reduce complexity, and digested by Lys-C followed by HPLC C4 isolation yielding the 157 amino acid MUC1 TR O-glycodomain fragment. All MUC1 O-glycoforms (Tn and T) showed a rather homogeneous mass comprising 32-35 HexNAc/HexHexNAc residues, and with 33 or 34 HexNAc/HexHexNAc being the most abundant peak. Panel b Site-specific O-glycopeptide LC-MS/MS analysis of MUC1 reporters after AspN digestion. Panel c MALDI-TOF profiling of released O-glycans. Structures of glycans are shown with symbols drawn according to the Symbol Nomenclature for Glycans (SNFG) format44. Note that colors are omitted, but illustrated HexNAc (open/grey), Hex, and sialic acid represent GalNAc/GlcNAc, Gal and NeuAc.

FIG. 8 Illustrates intact mass spectra of secreted mucin TR reporters, of which are classified as mucins (MUC2TR1, MUC2TR2mucinTR reporters designed from secreted mucins (MUC2TR#1, MUC2TR2MUC2#2, MUC5AC and MUC7) and classified as membrane-bound mucins (MUC13 and MUC21) produced in HEK293^{KO CIGALT1} (Tn).

FIG. 9 Shows that the glycoprotease StcE cleaves selective mucin TRs and O-glycoforms. Panel a Graphic depiction of the assay designs for enzyme assays with isolated and cell membrane bound mucin TR reporters. Panel b SDS-PAGE analysis of StcE digestion (dose titration) of isolated MUC2#1 (upper panel) and MUC5AC (lower panel) reporters produced in glycoengineered HEK293 cells (core2, Tn and STn O-glycoforms). Purified reporters (0.5 μg) were incubated for 2 h at 37° C., and gels visualized with Krypton fluorescent protein stain. Representative gels of three independent experiments are shown. Panel c Flow cytometry analysis of StcE cleavage of different TR reporters expressed stably as membrane bound proteins on the surface of HEK293^{WT} cells and detected by anti-FLAG antibody binding. Bar diagram shows average mean fluorescent intensities (MFI)±SEM. An artificially designed TR reporter with a single O-glycosite was used to serve as a control (ctrl) for the clusters and patterns of O-glycans found in human mucin TRs. Data from three independent experiments are shown. Panel d Flow cytometry analysis of StcE cleavage (dose titration) of MUC2#1 and MUC5AC membrane reporters stably expressed on glycoengineered HEK293 cells (core2, dST, mSTa, T, Tn, core3 and STn glycoforms). Note that core3 is represented as the core disaccharide (GlcNAcβ1-3GalNAcα1-O-Ser/Thr), but this is likely galactosylated and sialylated. Data are presented as average MFI±SEM of three independent experiments. Structures of glycans are shown with symbols drawn according to the Symbol Nomenclature for Glycans (SNFG) format⁴⁴. Note that colors are omitted, but illustrated HexNAc (open/grey), Hex, and sialic acid represent GalNAc/GlcNAc, Gal and NeuAc.

FIG. 10 Illustrates analysis of StcE activity with isolated secreted and cell membrane-bound mucin TR reporters. Panel a SDS-PAGE analysis of StcE (dose titration) digestion of secreted purified MUC7 TR reporters with different glycoforms. Panel b SDS-PAGE analysis of StcE digestion of MUC1 TR reporters with 1:10 ratio enzyme to substrate of core2 and Tn glycoforms. Panel c Flow cytometry analysis of membrane bound reporters illustrating the gating strategy for transiently expressed GFP-tagged mucin TR reporters in HEK293 cells. Gating for GFP positive cells correlates well with the population of cells labelled by the anti-FLAG mAb detecting surface located mucin TR reporters. Panel d Representative histograms of membrane MUC2#1, MUC5AC, MUC7 and MUC1 TR reporters expressed in HEK293WT cells by increasing concentrations of StcE as determined by staining with anti-FLAG mAb. Panel e Representative histograms show StcE-mediated cleavage of MUC2#1, MUC5AC, MUC7 and MUC1 TR reporters expressed by HEK293 cells with core2, diST, mSTa, T, Tn, core3 or STn glycosylation. Mock transfected cells and transfected, untreated cells are shown as control.

FIG. 11 Illustrates that the mucin binding properties of StcE is mediated by the X409 domain. Panel a Graphic depiction of the design of enzyme activity and binding assays performed. Panel b Schematic representation of expression constructs for full coding StcE^{WT}, StcE^{E447D}, X409 domain truncated StcE (StcE^{ΔX409}), and the isolated X409 domains±GFP (left). Flow cytometry analysis of cleavage (dose titration) of membrane bound MUC2#1 expressed in HEK293^{WT} cells by StcE^{WT}, StcE^{E447D}, Stc^{EΔΔX409}, and the X409 domain detected by anti-FLAG staining and flow cytometry (right). Representative data of three independent experiments with similar results are shown. Panel c Immunofluorescence staining of representative sections of normal colon stained with StcE, StcE^{E447D}, Stc^{EΔX409}, X409, and anti-MUC2 mAb (PMH1). Counterstained with DAPI (blue). Panel d Flow cytometry analysis of X409-GFP binding to HEK293WT cells stably expressing mucin TR reporters tagged with CFP (instead of GFP).

Average MFI±SEM values from three independent experiments are shown. Panel e Flow cytometry analysis of X409-GFP binding (1 μg/ml) to MUC2#1, MUC5AC, MUC7 and MUC1 reporters expressed on glycoengineered HEK293 cells (core2, diST, mSTa, T, Tn, core3, and STn). Structures of glycans are shown with symbols drawn according to the Symbol Nomenclature for Glycans (SNFG) format44. Note that colors are omitted but illustrated HexNAc (open/grey), Hex, and sialic acid represent GalNAc/GlcNAc, Gal and NeuAc.

FIG. 12 Illustrates that the StcE X409 domain binds mucins in situ. Panel a SDS-PAGE analysis of StcE and StcEΔX409 digestion (time course 1:200 ratio) of the MUC2#1 TR reporter expressed in HEK293WT. Panel b Representative fluorescence images of sections from normal colon (pretreated with neuraminidase) reacted with X409-GFP and anti-Tn-MUC2 (PMH1) mAb. Panel c Images of normal and neoplastic tissue microarray sections reacted with X409-GFP. Fluorescence is shown as white/light grey.

FIG. 13 Illustrates ELISA analysis of X409 binding to animal mucins (PSM, BSM, OSM) and select human mucin TRs. ELISA analysis with purified, secreted mucin reporters derived from the cell-based mucin display platform showing selective binding of X409 to purified porcine submaxillary mucin (PSM), Tn MUC5AC and Tn MUC21. No binding was observed for purified ovine submaxillary gland mucin (OSM/AOSM), bovine submaxillary mucin (BSM) and Tn/STn MUC1 (right).

FIG. 14 Illustrates the 3-D structure of StcE (PDB 3UJZ) and StcE protein sequence. Panel a The catalytic domain is shown (top) with the catalytic zinc shown (small circle). The X409 distinct module is shown in opposite the catalytic domain (bottom). Panel b Illustrates the amino acid sequence of Escherichia coli O157:H7 StcE with the signal peptide (not underlined), the catalytic domain (underlined), and the X409 module (underlined 2×). Panel c shows the nucleic acid sequence of Escherichia coli O157:H7 StcE with translation into amino acid sequence

FIG. 15 illustrates a phylogenic tree of the genes identified with related X409 binding modules including accession numbers and strain origin. The X409 module sequence of Escherichia coli O157:H7 StcE exhibits between 100% and 65% amino acid sequence identity to related modules found in Zn-metalloproteases with Pfam10462 domains. The X409 module sequence of Escherichia coli O157:H7 StcE exhibits 35-100% amino acid sequence identity with X409 modules found in other bacteria.

FIG. 16 Illustrates the natural sequence variation of the X409 binding module in different bacteria. Multiple sequence alignment of a sampling of over 60 sequences of related X409 binding modules with high conservation are shown with GenBank accession numbers and bacterial strain origin. Multiple sequence alignments were computed using Muscle⁴⁶ using default parameters. Rendering of the multiple alignment was done using ESPRIPT⁴⁷. The alignment is shown in FIGS. 16-1/2. The alignment shows that residue 3 (Cys/C) is the first fully conserved residue of the X409 sequence, and residue 96 (Val/V) is the last fully conserved, strongly suggesting that these residues confine the minimum binding module of X409. Residues 1-2 (Glu-Gly/EG) are semi-conserved among the 60 sequences and thus may contribute to the properties of X409. Similarly, residue 97 (Val/V) is semi-conserved, while residues 98-99 (Tyr-Lys/YK) are less conserved.

FIG. 17 Illustrates 456 amino acid sequences of X409 related protein modules with protein sequence accession and domain limits for each. StcE is indicated and a consensus sequence for all sequences is shown. The residues conserved across the multiple alignment are important for the fold and the binding function of these X409 related binding modules. The Figure includes five consecutive Figures split into two. Multiple sequence alignments were computed using Muscle46 using default parameters. Rendering of the multiple alignment was done using ESPRIPT⁴⁷. The alignment shows the same pattern of conserved X409 residues 1-3 and 96-97.

FIG. 18 illustrates the novel mucin-binding module designated HC1 CBM51 found in Clostridium perfringens LLY_N11 (GenBank accession number ATD49073.1), a sequence unrelated to the X409 mucin-binding module. Panel a The novel mucin-binding module is derived from the Clostridium perfringens gene accession no. ATD49073.1. Schematic representation of the expression construct for the coding protein is shown with the full coding sequence where the sequence of the CBM51 module is underlined. Panel b Fluorescence immunohistology shows selective binding of the CBM51 module to stomach mucin-producing cells and no binding to cells in colon sections. Panel c shows flow cytometry analysis of the CBM51 module to HEK293WT cells transiently expressing mucin TR reporters as indicated.

FIG. 19 illustrates the novel mucin-binding module designated HC7 X408-FN3-CBM5 found in Bacillus cereus K8 (GenBank accession number ASJ51756.1), a sequence unrelated to the X409 mucin-binding module. Panel a The novel mucin-binding module is derived from the Bacillus cereus K8 (Gene Bank accession number ASJ51756.1). Schematic representation of the expression construct for the full length protein is shown with the full coding sequence where the sequence of the X408-FN3-CBM5 module is underlined. Panel b Fluorescence immunohistology shows selective binding of the X408-FN3-CBM5 module to stomach mucin-producing cells and no binding to cells in colon sections. Panel c shows flow cytometry analysis of the X408-FN3-CBM5 module to HEK293WT cells transiently expressing mucin TR reporters as indicated.

FIG. 20 illustrates the novel mucin-binding module designated HC11 Bacon-Bacon-CBM32 from Bacteroides fragilis (GenBank accession number ASJ51756.1), a sequence unrelated to the X409 mucin-binding module. Panel a The novel mucin-binding module is derived from Bacteroides fragilis (GenBank accession number ASJ51756.1). Schematic representation of the expression construct for the full coding protein is shown with the full coding sequence where the sequence of the Bacon-Bacon-CBM32 module is underlined. Panel b Fluorescence immunohistology shows selective binding of the Bacon-Bacon-CBM32 module to stomach mucin-producing cells and no binding to cells in colon sections. Panel c shows flow cytometry analysis of the Bacon-Bacon-CBM32 module to HEK293WT cells transiently expressing mucin TR reporters as indicated.

FIG. 21 illustrates the novel mucin-binding module designated HC12 Bacteroides thetaiotaomicron (GenBank AAO79349 or WP_008764444), a sequence unrelated to the X409 mucin-binding module. Panel a The novel mucin-binding module is derived from Bacteroides thetaiotaomicron (GenBank AAO79349 or WP_008764444). Schematic representation of the expression construct for the full coding protein is shown with the full coding sequence where the sequence of the Bacon-CBM32 module is underlined. Panel b Fluorescence immunohistology shows selective binding of the Bacon-CBM32 module to stomach mucin-producing cells as well as cells in colon sections. Panel c shows flow cytometry analysis of the Bacon-CBM32 module to HEK293WT cells transiently expressing mucin TR reporters as indicated.

FIG. 22 illustrates determination of binding affinities of X409 towards mucin TRs and glycoforms by microscale thermophoresis (MST) assay. Panel a Schematic representation of MST assay with AlexaFluoro647 labelled MBP-X409. Panel b MST spectrum data with data fitting curve for different MUC5AC TR reporter and glycoforms as indicated. Tn; GalNAcα1-O-Ser/Thr, WT; wild type core2 sialylated O-glycans as produced in WT HEK293 cells. Panel c Calculated dissociation constants from Panel b. Two constants indicate biphasic binding states.

FIG. 23 illustrates binding properties of four representative X409 variant sequences from FIG. 16/17 by flow cytometry analysis. Panel a shows binding of X409 (E. coli 0157) and four related X409 modules from Vibrio anaquillarum (Gene Bank accession number AZS25716), Aeromonas hydrophilia(accession number QBX76946), Shewanella baltica OS223 (accession number ACK48812), and E. coli (AUM10835) towards MUC5Ac expressed on glycoengineered HEK293 cells with different O-glycans. E. coli (AUM10835) exhibits narrower glycan specificity without binding to In and STn glycoforms. Panel b shows binding of the same X409 modules to HEK293 core1 cells (expressing T O-glycans) transiently expressing 8 different mucin TR reporters as indicated.

FIG. 24 illustrates analysis binding properties of novel mucin-binding modules to different glycoforms of 12 human mucin TRs by flow cytometry. HC1 (CBM51 derived from Clostridium perfringens LLY_N11, access number ATD49073.1, see FIG. 18) binds all mucins with complex O-glycans but not Tn compared to X409 (StcE control).

FIG. 25 illustrates analysis binding properties of X409 mucin-binding modules from StcE to different glycoforms of 12 human mucin TRs by flow cytometry. HC7 (X408/FN3/CBM5 from Bacillus cereus K8, access number ASJ51756.1, see FIG. 19) binds selective mucins and complex O-glycans with improved selectivity compared to X409 (StcE control).

FIG. 26 illustrates analysis binding properties of X409 mucin-binding modules from StcE to different glycoforms of 12 human mucin TRs by flow cytometry. HC11 (2×Bacon/CBM32 from Bacteroides fragilis, access number QCT79445.1, see FIG. 20) binds mucins selectively with core1 T O-glycans but not Tn compared to X409 (StcE control).

FIG. 27 illustrates analysis binding properties of X409 mucin-binding modules from StcE to different glycoforms of 12 human mucin TRs by flow cytometry. HC12 (Bacon/CBM32 from Bacteroides thetaiotaomicron, access number AAO79349 or WP_008764444, see FIG. 21) binds mucins selectively with Tn O-glycans but not core1 O-glycans compared to X409 (StcE control).

FIG. 28 illustrates analysis binding properties of X409 mucin-binding modules from StcE to different glycoforms of 12 human mucin TRs by flow cytometry. HC5 (from Pedobacter steynii DX4, access number AOM76365.1) binds mucins selectively with Tn O-glycans but not core1 O-glycans compared to X409 (StcE control).

### DETAILED DESCRIPTION

It is an object of the present invention to provide novel mucin-binding peptides that exhibit select binding properties for mucins and O-glycodomains in glycoproteins and to provide mucin-binding targeting agents comprising such peptides.

It is an object of the present invention to provide novel mucin-binding peptides that exhibit select binding properties for mucins and mucin tandem repeat regions, which binding is not dependent on the structures of O-glycans attached to these.

It is an object of the present invention to provide novel mucin-binding peptides that exhibit select binding properties for mucins and mucin tandem repeat regions which binding is independent on the structures of the O-glycans attached to these mucins.

An object of the present invention relates to the mucin-binding properties of the X409 peptide module and related sequences and use of these to bind mucins for diagnosis of disease.

An object of the present invention relates to the mucin-binding properties of the X409 peptide module and related sequences and use of these to bind mucins for therapeutic purposes.

An object of the present invention relates to the mucin-binding properties of the X409 peptide module and related sequences and use of these for delivery of pharmacological agents to mucosal surfaces.

An object of the present invention relates to methods of using the X409 peptide module and other peptides and peptide modules to obtain mucin-binding properties for pharmaceutical formulations.

The present invention relates to a peptide or peptide module (designated X409) found in the Secreted Protease of C1 Esterase Inhibitor (StcE) bacterial protease and other bacterial proteins and peptide modules that have select mucin-binding properties.

It is an object of the present invention to provide related X409 binding modules with high sequence similarity within bacteria.

It is an object of the present invention to provide related X409 binding modules with lower sequence similarity within bacteria, such as 65% sequence identity or more, e.g. such as such as 70% or more, such as 80% or more, such as 85% or more, such as 90 percent or more, such as 95% or more, such as 96% or more, such as 97% or more, such as 98% or more, such 99% or more, such as 99.5% or more thereto.

The present invention provides solutions to the objects above.

The present invention provides the unique binding specificity of a small peptide module X409 with highly select binding to mucins with clusters of O-glycans attached.

The present invention provides the unique binding specificity of the small peptide module X409 with binding to mucins with clusters of O-glycans and with binding to such mucins with different types of O-glycan structures attached.

The present invention provides multiple X409-related mucin-binding peptide and peptide modules, and mucin-binding peptides and peptide modules that are not related to X409.

The present invention provides small peptide sequence modules with binding to select mucin tandem repeat regions and containing clusters of different types of O-glycans.

It is contemplated that mucin-binding targeting agents are for use in a mammal such as e.g. a human.

The mucin-binding targeting agents may be used as a medicament in a human or on an animal, such as in veterinary care or animal health.

### Definitions

The mucin-binding modules or polypeptides of the invention bind to densely O-glycosylated mucins and mucin-like glycoproteins, i.e. to clusters of O-glycans with 2,3,4,5,6,7, or 8 consecutive O-glycans attached to adjacent Ser/Thr residues often arranged in multiple consecutive patterns.

The term “isolated” as used herein in relation to peptides refers to amino acid sequences which have been taken out of their native environment. Thus, an isolated peptide is a non-native peptide which may be a part (or sub-sequence) of a larger peptide or protein. Isolated peptides are identified and selected based on their affinity for preferred mucins and used in mucin-binding targeting agents, which can be chimeric construct that may comprise also a payload. The chimeric constructs forming the mucin-binding targeting agents may be produced by recombinant expression in a host cell, such as a bacteria.

The term “binding affinity” is used herein to describe the strength of interaction between to binding partners, such as the mucin-binding targeting agent (or the isolated peptide) and a mucin, such as MUC5AC or MUC1. The binding affinity may be quantified by determination of the dissociation constant of said interaction. A low dissociation constant indicates a strong interaction (or binding).

The term payload as used herein refers to a moiety that is intended to be delivered to a tissue by the binding of a mucin-binding targeting agent as provided herein. The payload is thus attached to said mucin-binding targeting agent by a binding moiety.

The binding moiety may be e.g. a peptide linker, an ester, a lipid anchor, avidin, streptavidin, biotin, or another binding moiety such as an antibody, or a nanobody.

The binding moiety may be able to undergo in vivo acid hydrolysis or may comprise a protease site that can undergo cleavage to ensure the mucin-binding-domain is not delivered e.g. with a bioactive peptide to be taken up systematically.

The term payload may refer to an entire complex, such as a nanoparticle, liposome, vesicle, which contains a bioactive compound to be delivered or it may refer to a bioactive compound, stain, or e.g. a detectable marker. It is contemplated that when the payload is a liposome or another vesicle it may be attached to the mucin-binding targeting agent by a binding moiety in the form of a lipid anchor, or by a different moiety inserted into the liposome or vesicle. In the latter case the moiety is then bound or attached to mucin-binding targeting agent via the binding moiety.

When the payload is a liposome a bioactive peptide or protein, oligonucleotide, or other therapeutic agents such as a therapeutic peptide may be inside the liposome.

Therapeutic peptides may also be attached to the binding moiety, or may form chimeric proteins with the mucin-binding targeting agent, in which the binding moiety may be a peptide bond or a peptide linker.

Therapeutic peptides and proteins for use in or as payload may be selected from the group comprising:

Neuroendocrine protein 7B2, Acyl-CoA-binding domain-containing protein, Adrenomedullin, Proadrenomedullin NApelin-13 , Apelin, Gastrin-releasing peptide, Neuromedin-C, Neuromedin-B, Bradykinin, T-kinin, Calcitonin, Katacalcin, Calcitonin gene-related peptide 1, Calcitonin gene-related peptide 2, Islet amyloid polypeptide, CART Cocaine- and amphetamine-regulated, Cerebellin -4, Cerebellin-1, Cerebellin-2, Cerebellin-3, AL-11, Chromogranin-A, EA-92, ER-37, ES-43, GR-44, GV-19, LF-19, Pancreastatin, SS-18, Vasostatin, WA-8, WE-14, CCB peptide, GAWK peptide, Secretogranin, Secretoneurin, Kininogen, Big endothelin-1, Endothelin, Neuropeptide AF, Neuropeptide FF, Neuropeptide SF, Neuropeptide NPSF, Neuropeptide NPVF, Neuropeptide RFRP Prolactin-releasing peptide, Galanin, Galanin message-associated peptide, Galanin-like peptide, Cholecystokinin, Big gastrin, Gastrin, Gastric inhibitory polypeptide, Glicentin, Glicentin-related polypeptide, Glucagon, Glucagon-like peptide 1, Glucagon-like peptide 2, Oxyntomodulin, PACAP-related peptide, Pituitary adenylate cyclase-activating peptide 27, Pituitary adenylate cyclase-activating peptide 38, Secretin, Somatoliberin, Intestinal peptide PHM-27, Intestinal peptide PHV-42, Vasoactive intestinal peptide, GnRH-associated peptide 1, Gonadoliberin-1, Progonadoliberin-1, GnRH-associated peptide 2, Gonadoliberin-2, Progonadoliberin-2, Insulin-like growth factor I, Insulin-like growth factor Il, Preptin, Insulin A chain, Insulin B chain, Relaxin A chain, Relaxin B chain , Relaxin A chain, Relaxin B chain, Relaxin-3 A chain, Relaxin-3 B chain , Kisspeptin-10, Kisspeptin-13, Kisspeptin-14, Metastasis-suppressor KiSS-1, Metastin, Leptin, Melanin-concentrating hormone, Neuropeptide-glutamic acid-isoleucine, Neuropeptide-glycine-glutamic acid, Pro-MCH, Ghrelins, Obestatin, Motilin, Motilin-associated peptide, Promotilin, Adiponectin, Ubiquitin-like protein 5, Agouti-related protein, Nicotinamide phosphoribosyltransferase, Atrial natriuretic peptide ANP, Cardiodilatin-related peptide, Brain natriuretic peptide BNP , Cardiac natriuretic peptide CNP, Neurexophilin-1, Neurexophilin-2, Neurexophilin-3, Neurexophilin-4, Neuromedin-S, Neuromedin-U-25, Neuropeptide B-23, Neuropeptide B-29, Neuropeptide W-23, Neuropeptide W-30, Neuropeptide S, Large neuromedin N, Neuromedin N, Neurotensin, Tail peptide, C-flanking peptide of NPY, Neuropeptide Y, Pancreatic hormone, Pancreatic icosapeptide, Peptide YY, Nucleobindin-1, Nesfatin-1, Nucleobindin-2, Deltorphin I, Gamma-Lipotropin, γ-melanocyte-stimulating hormone, Alpha-neoendorphin, Beta-neoendorphin, Big dynorphin, Dynorphin A, Leu-enkephalin, Leumorphin, Rimorphin, Met-enkephalin, PENK, Synenkephalin, Neuropeptide 1, Neuropeptide 2, Nociceptin, Orexin-A, Orexin-B, Tuberoinfundibular peptide, Osteostatin, Parathyroid hormone-related protein, PTHrP, Beta-endorphin, Corticotropin, Corticotropin-like intermediary peptide, Lipotropin beta, Lipotropin gamma, Melanotropin alpha, Melanotropin beta, Melanotropin gamma, NPP, ProSAAS, Big LEN, Big PEN-LEN, Big SAAS KEP, Little LEN, Little SAAS, PEN, Resistin, Resistin-like beta, QRF-amide, Corticoliberin, Urocortin, Urocortin-2, Urocortin-3, Corticosteroid-binding globulin, Serpin, Angiotensin, Angiotensinogen, Cortistatin, Somatostatin, Prolactin, Substance P, C-terminal-flanking peptide, Neurokinin A, Neuropeptide K, Substance P, Neurokinin-B, Pro-thyrotropin-releasing hormone, Thyrotropin-releasing hormone, Urotensin, Neurophysin 1, Oxytocin, Arg-vasopressin, Copeptin, Neurophysin 2, Antimicrobial peptide VGF, Neuroendocrine regulatory peptide-1, Neuroendocrine regulatory peptide-2, Neurosecretory protein VGF.

The payload may be in the form of a peptide or protein or a part of a protein. The mucin-binding targeting agent and the payload form a chimeric protein. In such cases the binding moiety will be understood to be e.g. a peptide bond. Such protein or peptide payloads may be therapeutic peptides. It is also contemplated that the payload may be a receptor, a toxin, or a lectin-binding protein.

The payload may also be an enzyme, in which case the mucin-binding targeting agent may facilitate retention of the enzyme at the site of action, e.g. in the pancreas or the gut. This mode of action may be used for improving efficacy of the enzymes. Thus, enzymes include, but are not limited to, therapeutic and/or digestive enzymes. In particular, digestive enzymes targeted to the pancreas may be utilized for treatment of patients having their pancreas surgically removed. Enzymes targeted to the gut can improve feed digestion and nutrient uptake as the mucin-binding targeting agent ensures prolonged retention in the gut via specific binding to site-specific mucins carrying non-truncated sugars. The payload is not limited to any particular enzyme but may be any type of enzyme, including, but not limited to, proteases, lipases, phytases, amylase, xylanases, β-Glucanases, α-Galactosidases, mannanases, cellulases, hemicellulases, and pectinases.

It is to be understood that the mucin-binding targeting agent may be part of a fusion protein. Such fusion proteins may comprise a therapeutic agent, such as a drug, a therapeutic protein or a bioactive peptide. Fusion proteins protein can in this manner be utilized as delivery vehicles with enhanced retention at the targeted tissue, such as the nasal tissue, the pancreas or the gut.

The payload can also be a vaccine. By combining a vaccine with the mucin-binding targeting agent, the vaccine may be efficiently delivered to the mucosa. It is contemplated that delivery to the mucosa will improve the immunological protection provided by the vaccine. Without being bound by theory, herein is suggested that immunological protection can be enhanced by presentation through the mucosa to stimulate the mucosal IgA immunity.

Thus, an embodiment of the present invention relates to the mucin-binding targeting agent, wherein the payload is a vaccine, such as a viral vaccine. Accordingly, the payload may comprise one or more antigens. According, an embodiment of the present invention relates to the mucin-binding targeting agent, wherein the payload comprises one or more antigens selected from the group consisting of proteins, peptides, polypeptides or nucleic acids. In particular, the nucleic acids may be DNA or RNA, and analogues thereof. The proteins may in some variants of the payload be a glycoprotein or a polysaccharide.

A further embodiment of the present invention relates to the mucin-binding targeting agent, wherein the one or more antigens are virus-specific antigens. Yet another embodiment of the present invention relates to the mucin-binding targeting agent, wherein the virus-specific antigen originates from a virus selected from the group consisting of SARS-COV-2 virus, SARS-COV-1 virus, Corona virus, Adenovirus, Norovirus, Papillomavirus, Polyomavirus, Herpes simplex virus (HSV), Alpha herpesvirinae human herpesvirus 1, 2, 3, Human gamma herpesvirus 4, 8 (Kaposi sarcoma), Betaherpesvirinae 5, 6, 7, Varicella zoster virus (VZV), Epstein-Barr virus (EBV), Cytomegalovirus (CMV), Picornavirus, Enterovirus, Rhinovirus, Hepatovirus, Cardiovirus, Aphthovirus, Coxsackie virus, Echovirus, Paramyxovirus, Measles virus, Parainfluenza virus, Mumps virus, Respiratory syncytial virus (RSV), Metapneumovirus, Nipah virus, Hendra viruses, Orthomyxoviruses, Influenza virus, Rhabdovirus, Filovirus, Marburg virus, Ebola virus, Bornavirus, Rabies virus, Reovirus, Rotavirus, Coltivirus, Orbivirus, Norwalk virus, Calicivirus, Rubella virus, Togavirus, Flavivirus, Arbovirus, Bunyavirus, Arena virus, Poxvirus, Parvovirus, Retrovirus, Human immunodeficiency virus (HIV), Human-T cell leukemia virus, and Hepatitis A, B, C, D, G, and E viruses.

A still further embodiment of the present invention relates to the mucin-binding targeting agent, wherein the one or more antigens originates from SARS-COV-2 virus.

It is contemplated that the mucin-binding targeting agent or part thereof may be expressed recombinantly. Thus, fusion proteins comprising the mucin-binding targeting agent may be expressed recombinantly.

It is contemplated that the mucin-binding targeting agents of the invention are catalytically inactive against mucins, i.e. they lack glycomucinase activity.

The payload may also be a radionuclide or a radiopeptide. The payload may be a therapeutic agent for use in the treatment of a cancer. It is contemplated that cancers affect the expression of mucins and therefore compositions according to the present invention may be used to deliver therapeutic agents to a cancer tissue with abnormal mucin expression,

The payload may also be a stain or a detectable marker, such as a chromophore, fluorophore, or radionuclide, or other detectable markers. Examples of detectable markers include nanodots and nanoparticles such as colloidal gold, and fluorescent proteins such as GFP. Such payloads enable the use of mucin-binding targeting agents according to the present invention for use in vitro or in vivo for immunological, histological, and/or diagnostic purposes. It is contemplated that such use can be for detecting normal and abnormal mucin-expressing tissue and may therefore be used e.g. to discern healthy and/or diseased tissue, such as in cancers and/or neoplasia of mucin expressing tissue, e.g. in the colon, stomach, pancreas, mammary, fallopian tube or other epithelial tissue. It is contemplated that such use is also for cancers and/or neoplasia epithelial and non-epithelial tissue where mucin-expression is abnormal, e.g. where mucin expression is absent or low in the non-diseased state but where mucin is expressed in the diseased state.

Accordingly, an embodiment of the present invention relates to the mucin-binding targeting agent as described herein, wherein the payload is selected from the group consisting of a therapeutic agent, an enzyme, a vaccine, a peptide hormone, a small molecule drug, a detectable marker, nanoparticle, liposome, vesicle and a stain.

The mucin binding targeting agent may be used as a medicament, and may be comprised in a composition. Such a composition may further comprise a pharmaceutically acceptable excipient and/or a pharmaceutically acceptable carrier. The compositions may be combined with excipients or coatings to form drug delivery formulations. Drug delivery formulations may be in a form of suspensions, tablets, capsules, gels, suppositories. The formulations may be oral suspensions to induce a rapid effect in combination with prolonged release. The formulation may be packaged in an excipient such as an enteric coating or a shell. The formulation may also include other mucoadhesive materials to enhance retention within the gastrointestinal tract. Compositions according to the present invention may be for oral, rectal, vaginal, buccal, ocular, nasal, or inhalation administration.

The aforementioned compounds of the invention or a formulation thereof may be administered by any conventional method including oral, and parenteral (e.g., subcutaneous or intramuscular) injection. The treatment may consist of a single dose or a plurality of doses over a period of time.

Whilst it is possible for a compound of the invention to be administered alone, it is preferable to present it as a pharmaceutical formulation, together with one or more acceptable carriers. The carrier(s) must be “acceptable” in the sense of being compatible with the compound of the invention and not deleterious to the recipients thereof. Typically, the carriers will be water or saline which will be sterile and pyrogen free.

In preferred embodiments, the mucin-binding targeting agent is administered to a subject or patient at a clinically relevant dose.

In yet other embodiments, suitable routes of administration are considered to be enteral administration, topical administration, and parenteral administration.

By enteral administration, we include methods including but not limited to oral administration, rectal administration, sublingual administration, sublabial administration, and buccal administration. Forms suitable for such administration include but are not limited to pills, tablets, osmotic controlled release capsules, solutions, softgels, suspensions, emulsions, syrups, elixirs, tinctures, hydrogels, ointments, suppositories, enemas, murphy drip, and nutrient enemas.

By topical administration, we include methods including but not limited to transdermal administration, vaginal administration, ocular administration, and nasal administration. Forms suitable for such administration include but are not limited to aerosols, creams, foams, gels, lotions, ointments, pastes, powders, shake lotions, solids (e.g., suppositories), sponges, tapes, tinctures, topical solutions, drops, rinses, sprays, transdermal patches, and vapors.

By parenteral administration, we include methods including but not limited to injection, insertion of an indwelling catheter, transdermal, and transmucosal administration. Such administration routes include but are not limited to epidural administration, intracerebral administration, intracerebroventricular administration, epicutaneous administration, sublingual administration, extra-amniotic administration, intra-arterial administration, intra-articular administration, intracardiac administration, intracavernous administration, intralesional administration, subcutaneous administration, intradermal administration, intralesional administration, intramuscular administration, intraosseous administration, intraperitoneal administration, intrathecal administration, intrauterine administration, intravaginal administration, intravesical administration, intravitreal administration, subcutaneous administration, transdermal administration, perivascular administration, and transmucosal administration. In particular, we include methods of administration including but not limited to epidural injection, intracerebral injection, intracerebroventricular injection, sublingual injection, extra-amniotic injection, intra-arterial injection, intra-articular injection, intracardial injection, intrapericardial injection, intracavernous injection, subcutaneous injection, intradermal injection, intramuscular injection, intraosseous injection, intraperitoneal injection, intrathecal injection, intrauterine injection, intravesical injection, intravitreal injection, subcutaneous injection, and perivascular injection.

The mucin-binding targeting agents of the invention and/or compositions comprising such agents may be for use as a medicament.

The mucin-binding targeting agents of the invention and/or compositions comprising such agents may be for use in the treatment of a disease, illness, or disorder in a subject.

Disease, illness, or disorder to be treated may be selected from the group of inflammatory, immunological, endocrine, or metabolic disorders such as obesity or may be neurological, psychological or psychiatric or mood disorders, or disorders of the nervous system, or sexual disorders including reproductive disorders and disorders of the genital system , or may be neoplastic disorders such as cancers. Also contemplated are disorders involving dysfunction of mucous tissue or dysfunction of epithelial tissue, including disorders, diseases, and illnesses of the gastrointestinal tract, nasal disorders, disorders and diseases of the eye, myopathy, obesity, anorexia, weight maintenance, diabetes, disorders associated with mitochondrial dysfunction, genetic disorders, cancer, heart disease, inflammation, disorders associated with the immune system, infertility, disease associated with the brain and/or metabolic energy levels.

Provided herein are also methods of delivery of one or more payloads to a tissue in a subject, wherein the tissue expresses one or more of MUC2, MUC5AC, MUC5B, MUC21. Such methods comprise administering to the subject a pharmaceutical composition comprising a mucin-binding targeting agent in the form of a peptide as provided herein and a payload bound to the polypeptide. The tissue may be located in the gastrointestinal tract, or may be epithelial or non-epithelial tissue located elsewhere. It is contemplated that the binding of the payload to the agent may be via the binding moiety. The binding moiety may be able to release the payload in vivo, such as at the target tissue, e.g. by being able to undergo acid hydrolysis or cleavage by an enzyme such as a protease.

The mucin-binding targeting agents described herein are advantageous in that they display a very distinct selectivity for specific mucins which allows precision targeting to desired tissues with reduced off-targeting and therefore less adverse effects. In particular, the mucin-binding targeting agents have low affinity for MUC1 compared to desired mucins, such as MUC5AC which is highly expressed in the gastrointestinal tract and the respiratory mucosal surfaces.

Additionally, the mucin-binding targeting agents display very high affinity for selected mucins that are orders of magnitude higher than traditional glycan-binding proteins, including lectins, that bind to sugars. This strong interaction with the target mucin facilitates prolonged retention of the mucin-binding targeting agent and any associated payload at a desired site of action.

The mucin-binding targeting agents of the invention may comprise an isolated X409 peptide according to SEQ ID NO:1, or a sequence having a certain sequence identity thereto. Such sequence identity may be e.g. 65% or more, such as 70% or more, such as 80% or more, such as 85% or more, such as 90 percent or more, such as 95% or more, such as 96% or more, such as 97% or more, such as 98% or more, such 99% or more, such as 99.5% or more. Sequence identity may be calculated using techniques known in the art, such as those of Example 4.

In some embodiments of the invention, the mucin-binding targeting agent consist of the isolated X409 peptide according to SEQ ID NO:1 or a sequence with at least 75% sequence identity to SEQ ID NO:1, such as at least 80% sequence identity to SEQ ID NO:1, such as at least 90% sequence identity to SEQ ID NO:1, such as at least 95% sequence identity to SEQ ID NO:1.

The X409 peptide may come from different bacterial source as this particular domain shares a high degree of homology across species. Thus, an embodiment of the present invention relates to the mucin-binding targeting agent, wherein the isolated peptide comprises an X409 peptide according to SEQ ID NO:1 or a X409 peptide derived from E. coli, A. Hydrophilia, or S. baltica having at least 75% sequence identity to SEQ ID NO: 1.

Another embodiment of the present invention relates to the mucin-binding targeting agent, wherein the isolated peptide is a X409 peptide selected from the group consisting of:

i) SEQ ID NO: 1, SEQ ID NO: 73, SEQ ID NO:74, and SEQ ID NO:135, and

ii) isolated peptides comprising an amino acid sequence having at least 90% sequence identity to any one of SEQ ID NO: 1, SEQ ID NO: 73, SEQ ID NO:74, or SEQ ID NO:135.

A preferred embodiment of the present invention relates to the mucin-binding targeting agent, wherein the isolated peptide comprises SEQ ID NO:135 (E. coli (accession number AUM10835)) or an isolated peptide comprising an amino acid sequence having at least 90% sequence identity to SEQ ID NO:135.

Another preferred embodiment of the present invention relates to the mucin-binding targeting agent, wherein the isolated peptide comprises SEQ ID NO:73 (Shewanella baltica OS223 (accession number ACK48812)) or an isolated peptide comprising an amino acid sequence having at least 90% sequence identity to SEQ ID NO:73.

A further preferred embodiment of the present invention relates to the mucin-binding targeting agent, wherein the isolated peptide comprises SEQ ID NO:74 (Aeromonas hydrophilia (accession number QBX76946)) or an isolated peptide comprising an amino acid sequence having at least 90% sequence identity to SEQ ID NO:74.

The mucin-binding targeting agents of the invention may comprise an isolated X409 peptide according to any one of SEQ ID NO:73, SEQ ID NO:74, or SEQ ID NO:135, or a sequence having a certain sequence identity thereto. Such sequence identity may be e.g. 65% or more, such as 70% or more, such as 80% or more, such as 85% or more, such as 90 percent or more, such as 95% or more, such as 96% or more, such as 97% or more, such as 98% or more, such 99% or more, such as 99.5% or more.

An embodiment of the present invention relates to the mucin-binding targeting agent, wherein the isolated peptide is a X409 peptide selected from the group consisting of:

i) SEQ ID NO: 100, SEQ ID NO: 109, SEQ ID NO: 165, SEQ ID NO: 174, SEQ ID NO:450, SEQ ID NO:609, SEQ ID NO:906, SEQ ID NO: 1066, and

ii) isolated peptides comprising an amino acid sequence having at least 90% sequence identity to any one of SEQ ID NO: 100, SEQ ID NO: 109, SEQ ID NO: 165, SEQ ID NO: 174, SEQ ID NO:450, SEQ ID NO:609, SEQ ID NO:906, and SEQ ID NO: 1066.

Another embodiment of the present invention relates to the mucin-binding targeting agent, wherein the isolated peptide comprises SEQ ID NO:109 (Vibrio anaquillarum (Gene Bank accession number AZS25716)) or an isolated peptide comprising an amino acid sequence having at least 90% sequence identity to SEQ ID NO:109.

It is to be understood, that these mucin-binding targeting agents may comprise or be attached to a payload as described herein. This can be in form of a fusion protein or as a conjugate or complex with another particle or vehicle, such as a lipid particle.

The mucin-binding targeting agents of the invention may alternatively comprise an isolated peptide according to any one of SEQ ID NO: 2 to 5, or a sequence having a certain sequence identity thereto. Such sequence identity may be e.g. 65% or more, such as 70% or more, such as 80% or more, such as 85% or more, such as 90 percent or more, such as 95% or more, such as 96% or more, such as 97% or more, such as 98% or more, such 99% or more, such as 99.5% or more. Sequence identity may be calculated using techniques known in the art, such as those of Example 4.

### Experimental Procedures

### Methods

### Cell Culture

HEK293^{WT} (ECACC 85120602) and all isogenic clones were cultured in DMEM (Sigma-Aldrich) supplemented with 10% heat-inactivated fetal bovine serum (Sigma-Aldrich) and 2 mM GlutaMAX (Gibco) in a humidified incubator at 37° C. and 5% CO2. The glycoengineered isogenic HEK293 cells used in this study part of the previously reported cell-based glycan array resource^{36,37}.

### Gene Engineering

CRISPR/Cas9 KO was performed using the GlycoCRISPR resource containing validated gRNAs libraries for targeting of all human glycosyltransferases⁴⁷, and site-directed KI was performed using a modified ZFN ObLigaRe targeted KI strategy, as previously described^{48,49}. In brief, HEK293 cells grown in 6-well plates (NUNC) to ˜70% confluency were transfected for CRISPR/Cas9 KO with 1 mg of gRNA and 1 mg of GFP-tagged Cas9-PBKS and for targeted KI with 0.5 mg of each ZFN-tagged with GFP/Crimson targeted to the safe-harbor AAVS1 site and 1 ug of respective donor plasmid. 24 h post-transfection, cells were bulk-sorted based on GFP expression by FACS (SONY SH800). After one week of culture, the bulk-sorted cells were single cell-sorted into 96-well plates and KO clones were screened for by Indel Detection by Amplicon Analysis (IDAA) as described⁵⁰, and gene KO of final clones was verified by Sanger sequencing. The allelic insertion status of KI clones was screened by junction PCR with a primer pair covering the junction area between donor plasmid and the AAVS1 locus, and a primer pair flanking the targeted KI locus.

### Human Mucin TR Reporters

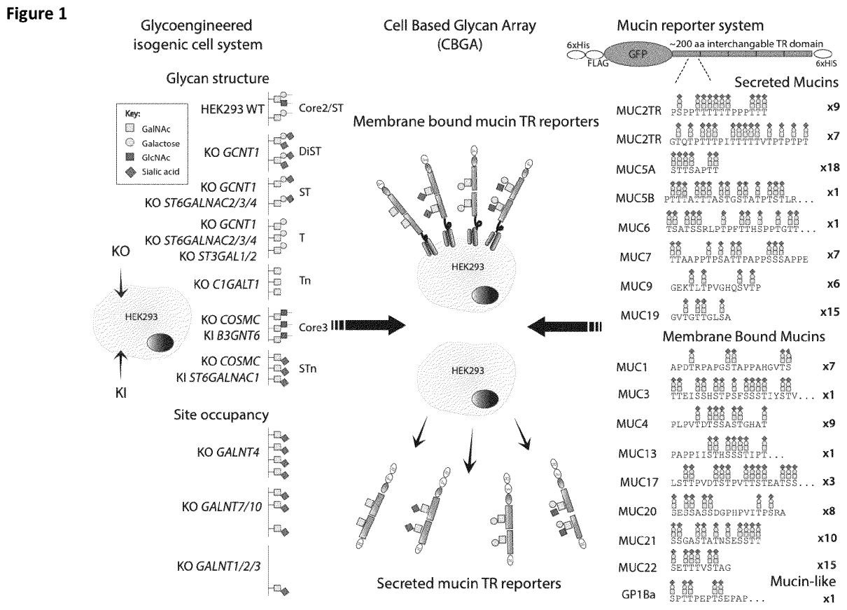

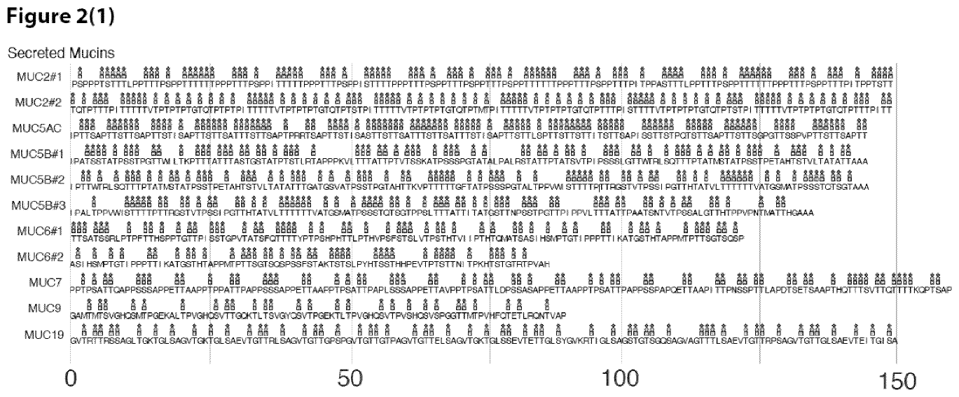

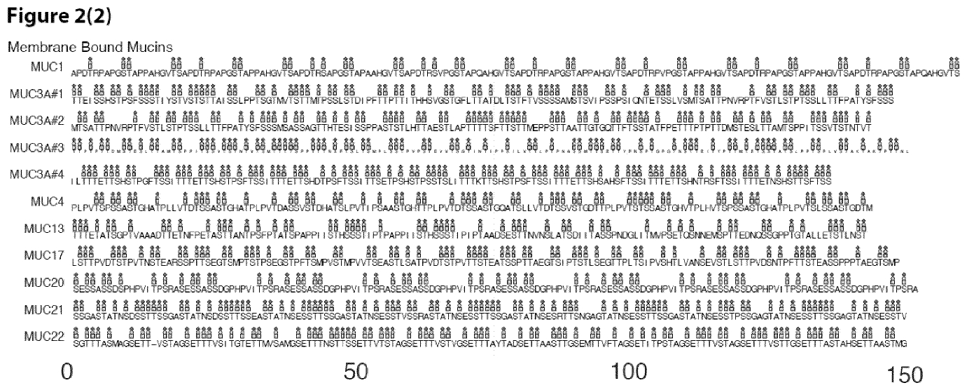

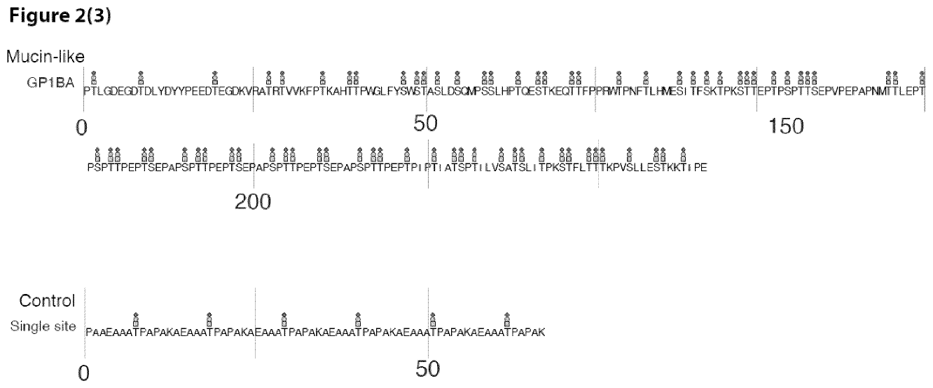

Transmembrane and secreted mucin TR reporter expression constructs were designed as previously described by use of exchangeable inserts of 150-200 amino acids derived from the TR regions of human mucins (FIG. 1 and Table 1)³⁶. The secreted TR constructs contain NotI/XhoI restriction sites and a 6xHis tag STOP encoding ds oligo (5′-GCGGCCGCCCATCACCACCATCATCACTGATAGCGCTCGAG-3′, NotI/XhoI restriction sites underlined). We also included a TR reporter design containing six 11-mer sequences with a single O-glycosylation site (AEAAATPAPAKₙ₌₆) to serve as control for the patterns of O-glycans found in mucin TRs (FIG. 2).

Transient Transfection with Mucin TR Reporters

Transmembrane GFP-tagged mucin TR reporter constructs were transiently expressed in engineered HEK293 cells. Briefly, cells were seeded in 24-wells (NUNC) and transfected at ˜70% confluency with 0.5 μg of plasmids using Lipofectamine 3000 (Thermo Fisher Scientific) following the manufacturer's protocol. Cells were harvested 24 h post-transfection and used for assays followed by flow cytometry analysis.

### Production and Purification of Recombinant Mucin TR Reporters

The secreted reporter constructs were stably expressed in isogenic HEK293-6E cell lines selected by two weeks of culture in the presence of 0.32 μg/mL G418 (Sigma-Aldrich) and two rounds of FACS enrichment for GFP expression. A stable pool of cells was seeded at a density of 0.25×10⁶ cells/ml and cultured for 5 days on an orbital shaker in F17 medium (Gibco) supplemented with 0.1 Kolliphor P188 (Sigma-Aldrich) and 2% Glutamax. Culture medium containing secreted mucin TR reporter was harvested (3,000×g, 10 min), mixed 3:1 (v/v) with 4× binding buffer (100 mM sodium phosphate, pH 7.4, 2 M NaCl), and run through a nickel-nitrilotriacetic acid (Ni-NTA) affinity resin column (Qiagen), pre-equilibrated with washing buffer (25 mM sodium phosphate, pH 7.4, 500 mM NaCl, 20 mM imidazole). The column was washed multiple times with washing buffer and mucin TR reporter was eluted with 200 mM imidazole. Eluted fractions were analyzed by SDS-PAGE and fractions containing the mucin TR reporter were desalted followed by buffer exchange to MiliQ using Zeba spin columns (Thermo Fisher Scientific). Yields were quantified using a Pierce™ BCA Protein Assay Kit (Thermo Fisher Scientific) following the manufacturer's instructions and NuPAGE Novex Bis-Tris (4-12%, Thermo Fisher Scientific) Coomassie blue analysis.

### O-Glycoprofiling

HPLC (C4) purified mucin TR reporters (10 μg) were incubated in 0.1M NaOH and 1M NaBH4 at 45° C. for 16 h. Released O-glycan alditols were desalted by cation-exchange chromatography (Dowex AG 50W 8X). Borate salts were converted into methyl borate esters by adding 1% acetic acid in methanol and evaporated under N₂ gas. Desalted O-glycan alditols were permethylated (in 150 μL DMSO, ˜20 mg NaOH powder, 30 μl methyl iodide) at room temperature for 1 h. The reaction was terminated by addition of 200 μL ice-cold MQ water followed by the addition of ˜200 μL chloroform. The organic phase was washed 5 times with 1 mL MQ water and evaporated under N₂ gas. Permethylated O-glycans were purified by custom Stage Tips (C18 sorbent from Empore 3 M) and eluted in 20 mL 35% (v/v) acetonitrile, of which 1 μl was co-crystalized with 1 μL DHB matrix (10 mg/ml in 70% acetonitrile, 0.1% TFA, 0.5 mM sodium acetate) before positive mode MALDI-TOF analysis.

### Isolation of Mucin TR O-glycodomains

Ni-chromatography purified intact mucin TR reporters (50 μg) were digested with 1 μg Lys-C (Roche) at a 1:35 ratio at 37° C. for 18 h in 50 mM ammonium bicarbonate buffer (pH 8.0). After heat inactivation at 98° C. for 15 min, reactions were dried by speed vac and desialylated with 40 mU C. perfringens neuraminidase (Sigma-Aldrich) for 5 hrs at 37° C. in 65 mM sodium acetate buffer (pH 5.0). This step was omitted for reporters expressed in HEK293^{KO COSMC} (Tn glycoforms). Samples were heat inactivated at 98° C. for 15 min and dried. For intact MS analysis samples were separated on by C4 HPLC (Aeris™ C4, 3.6 μm, 200 a, 250×2.1 mm, Phenomenex) using a 0-100% gradient of 90% acetonitrile in 0.1% TFA. Fractions containing the released TR O-glycodomains were verified by ELISA with lectins or mAbs, dried and resuspended in 20 μl of 0.1% FA for intact mass analysis. For bottom up analysis of the MUC1 reporter, samples (20 μg) were further digested 2× with 0.67 μg Endo-AspN at a 1:35 ratio for 18 hrs at 37° C. in 100 mM Tris-HCL (pH 8.0). After inactivation by the addition of 1 mL of concentrated TFA, samples were desalted using custom Stage Tips (C18 sorbent from Empore 3 M) and analyzed by LC-MS/MS.

### O-glycopeptide Bottom Up Analysis of Mucin TRs

LC MS/MS analysis was performed on EASY-nLC 1200 UHPLC (Thermo Fisher Scientific) interfaced via nanoSpray Flex ion source to an Orbitrap Fusion Lumos MS (Thermo Fisher Scientific). Briefly, the nLC was operated in a single analytical column set up using PicoFrit Emitters (New Objectives, 75 mm inner diameter) packed in-house with Reprosil-Pure-AQ C18 phase (Dr. Maisch, 1.9-mm particle size, 19-21 cm column length). Each sample was injected onto the column and eluted in gradients from 3 to 32% B for glycopeptides, and 10 to 40% for released and labeled glycans in 45 min at 200 nL/min (Solvent A, 100% H2O; Solvent B, 80% acetonitrile; both containing 0.1% (v/v) formic acid). A precursor MS1 scan (m/z 350-2,000) of intact peptides was acquired in the Orbitrap at the nominal resolution setting of 120,000, followed by Orbitrap HCD-MS2 and ETD-MS2 at the nominal resolution setting of 60,000 of the five most abundant multiply charged precursors in the MS1 spectrum; a minimum MS1 signal threshold of 50,000 was used for triggering data-dependent fragmentation events. Targeted MS/MS analysis was performed by setting up a targeted MSn (tMSn) Scan Properties pane.

### Intact Mass Analysis of Mucin TRs

Samples were analyzed by EASY-nLC 1200 UHPLC (Thermo Scientific Scientific) interfaced via nanoSpray Flex ion source to an on OrbiTrap Fusion/Lumos instrument (Thermo Scientific Scientific) using “high” mass range setting in m/z range 700-4000. The instrument was operated in “Low Pressure” Mode to provide optimal detection of intact protein masses. MS parameters settings: spray voltage 2.2 kV, source fragmentation energy 35 V. All ions were detected in OrbiTrap at the resolution of 7500 (at m/z 200). The number of microscans was set to 20. The nLC was operated in a single analytical column set up using PicoFrit Emitters (New Objectives, 75 mm inner diameter) packed in-house with C4 phase (Dr. Maisch, 3.0-mm particle size, 16-20 cm column length). Each sample was injected onto the column and eluted in gradients from 5 to 30% B in 25 min, from 30 to 100% B in 20 min and 100% B for 15min at 300 nL/min (Solvent A, 100% H2O; Solvent B, 80% acetonitrile; both containing 0.1% (v/v) formic acid).

### Data Analysis

Glycopeptide compositional analysis was performed from m/z features extracted from LC-MS data using in-house written SysBioWare software⁵¹. For m/z feature recognition from full MS scans Minora Feature Detector Node of the Proteome discoverer 2.2 (Thermo Fisher Scientific) was used. The list of precursor ions (m/z, charge, peak area) was imported as ASCII data into SysBioWare and compositional assignment within 3 ppm mass tolerance was performed. The main building blocks used for the compositional analysis were: NeuAc, Hex, HexNAc, dHex and the theoretical mass increment of the most prominent peptide corresponding to each potential glycosites. Upon generation of the potential glycopeptide list each glycosite was rank for the top 10 most abundant candidates and each candidate structure was confirmed by doing targeted MS/MS analysis followed by manual interpretation of the corresponding MS/MS spectrum. For intact mass analysis raw spectra were deconvoluted to zero-charge by BioPharma Finder Software (Thermo Fisher Scientific, San Jose) using default settings. Glycoproteoforms were annotated by in-house written SysBioWare software⁵¹ using average masses of Hexose, N-acetylhexosamine, and the known backbone mass of mucin TR reporter increment (MUC1, MUC2, MUC7, etc)

### Cell Binding Assays

For lectin staining HEK293 cells transiently expressing mucin TR reporters were incubated on ice or at 4° C. with biotinylated PNA, VVA (Vector Laboratories) or Pan-lectenz (Lectenz Bio) diluted in PBA (1× PBA containing 1% BSA (w/v)) for 1 h, followed by washing and staining with Alexa Fluor 647-conjugated streptavidin (Invitrogen) for 20 min. Stainings with mAbs specific to mucin glycoforms produced in mice was performed by incubating cells for 30 min at 4° C. with supernatant harvested from the respective hybridoma followed by staining with FITC-conjugated polyclonal rabbit anti-mouse Ig (Dako). Cells were stained with GST-tagged streptococcal adhesins at different concentrations diluted in PBA for 1 h on ice, followed by incubation with rabbit polyclonal anti-GST antibodies (Thermo Fisher) for 1 h and subsequent staining with Alexa Fluor 647 conjugated goat anti-rabbit IgG (Thermo Fisher) for 1 h. All cells were resuspended in PBA for flow cytometry analysis (SONY SA3800).

### ELISA

ELISA assays were performed using MaxiSorp 96-well plates (Nunc) coated with dilutions of purified mucin TR reporters starting from 100 ng/ml or fractions derived from C4 HPLC incubated o/n at 4° C. in 50 ml carbonate-bicarbonate buffer (pH 9.6). Plates were blocked with PLI-P buffer (PO₄, Na/K, 1% Triton-X100, 1% BSA, pH 7.4) and incubated with mAbs (undiluted culture sups or as indicated) or biotinylated-lectins (Vector Laboratories and Lectenz Bio) for 1 h at RT, followed by extensive washing with PBS containing 0.05% Tween-20, and incubation with 50 ml of 1 ug/mL HRP conjugated anti mouse Ig (Dako) or 1 ug/mL streptavidin-conjugated HRP (Dako) for 1 h. Plates were developed with TMB substrate (Dako) and reactions were stopped by addition of 0.5 M H2SO4 followed by measurement of absorbance at 450 nm.

### StcE Proteolytic Activity and Binding Assays

Recombinant StcE, StcE^{E447D}, StcE^{ΔX409}, and X409 were produced in E. coli similarly as reported previously²⁴. Enzyme assays with purified intact mucin reporters (500 ng) were performed by incubating serial dilutions of StcE for 2 h at 37° C. in 20 mL reactions in 50 mM ammonium bicarbonate buffer, and reactions were stopped by heat-inactivation at 95° C. for 5 min. Samples were run on NuPAGE Novex gels (Bis-Tris 4-12%) at 100 V for 1 h followed by staining with Krypton Fluorescent Protein Stain (Thermo Fisher Scientific) according to the manufacturer's instructions. Gels were imaged using an ImageQuant LAS 4000 system (GE Healthcare). Cell-based activity assays were performed with HEK293 cells transiently expressing mucin TR reporters incubated with serial dilutions of StcE in PBA at 37° C. After 1 h, cells were washed with PBA, stained with APC-conjugated anti-FLAG antibody (BioLegend) for 30 min at 4° C., and washed cells were analyzed by flow cytometry. Mean fluorescent intensity of anti-FLAG binding to GFP positive (transfected) and negative (untransfected) populations was quantified as using FlowJo (FlowJo LLC). For cell-binding assays with X409-GFP, HEK293 cells expressing CFP-tagged membrane TR reporters were used. Cells were incubated with different concentrations of X409-GFP for 1 h at 4° C. followed by staining with APC-conjugated anti-FLAG antibody. X409-GFP binding to anti-FLAG positive cells was quantified using FlowJo software. For histology analysis, deparrafinized tissue microarray sections52 were microwave treated for 20 min in sodium citrate buffer (10 mM, pH 6.0) for antigen retrieval followed by 1 h blocking with 1× PBS containing 5% BSA (w/v). Sections were incubated o/n at 4° C. with 5 μg/ml 6xHis-tagged StcE or StcE^{ΔX409} followed by washing and subsequent 1 h incubation with first mouse anti-6xHis antibody (Thermo Fisher) and second AF488-conjugated rabbit anti-mouse IgG (Invitrogen).

Sections stained with 2 μg/ml GFP-X409 were optionally sialidase treated and co-stained with mouse anti-MUC2 (PMH1) and donkey-anti mouse IgG Cy3 (Jackson ImmunoResearch). All samples were mounted with ProLong Gold Antifade Mountant with DAPI (Molecular Probes) and imaged using a Zeiss microscopy system followed by analysis with ImageJ (NIH).

Data analysis

Glycopeptide compositional analysis was performed from m/z features extracted from LC-MS data using in-house written SysBioWare software⁵¹. For m/z feature recognition from full MS scans Minora Feature Detector Node of the Proteome discoverer 2.2 (Thermo Fisher Scientific) was used. The list of precursor ions (m/z, charge, peak area) was imported as ASCII data into SysBioWare and compositional assignment within 3 ppm mass tolerance was performed. The main building blocks used for the compositional analysis were: NeuAc, Hex, HexNAc, dHex and the theoretical mass increment of the most prominent peptide corresponding to each potential glycosite. Upon generation of the potential glycopeptide list each glycosite was ranked for the top 10 most abundant candidates and each candidate structure was confirmed by doing targeted MS/MS analysis followed by manual interpretation of the corresponding MS/MS spectrum. For intact mass analysis raw spectra were deconvoluted to zero-charge by BioPharma Finder Software (Thermo Fisher Scientific, San Jose) using default settings. Glycoproteoforms were annotated by in-house written SysBioWare software⁵¹ using the average masses of Hexose, N-acetylhexosamine, and the known backbone mass of mucin TR reporter increment.

TABLE 1
Amino Acid sequences of Reporter Constructs Used, Related to Figure 1 and 2

| Gene | Uniprot | | Postion in | length | |
|---|---|---|---|---|---|
| (HGNC) | ID | | Uniprot ID | (aa) | Sequence |
| MUC1 | P15941 | | 121-260 | 140 | APDNKPAPGSTAPPAHGVTS APDTRPAPGSTAPPAHGVTS APDTRPAPGSTAPPAHGVTS APDTRPAPGSTAPPAHGVTS |
| | | | | | APDTRPAPGSTAPPAHGVTS APDTRPAPGSTAPPAHGVTS APDTRPAPGSTAPPAHGVTS |
| MUC2 | A0A3S8T | #1 | 1406-1554 | 149 | PSPPPTST TTLPPTTT PSPPTTTT TTPPPTTT PSPPITTT TTPPPTTT PSPPISTT TTPPPTTT PSPPTTT PS PPTTT |
| | MF2* | #2 | 1916-2064** | 152 | PSPPTTTT TTPPPTTT PS PPTTT PITPPAST TTLPPTTT PSPPTTTT TTPPPTTT PSPPTTT PITPPTST T |
| | | | | | PTQTPTTTPITTTTTVTPTPTPT GTQTPTPTPITTTTTVTPTPTPT GTQTPTSTPITTTTTVTPTPTPT |
| | | | | | GTQTPTMTPITTTTTVTPTPTPT GTQTPTTTPISTTTTVTPTPTPT GTQTPTSTPITTTTTVTPTPTPT GTQTPTTTPITTAA |
| MJC3A | Q02505 | #1 | 391-538 | 148 | TTEISSHSTPSFSSSTIYSTVSTSTTAISSLPPTSGTMVTSTTMTPSSLSTDIPFTTPTTITHHSVGSTGFLTTATDLTSTFTVSSSSAMSTSVIP |
| | | | | | SSPSIQNTETSSLVSMTSATTPNVRPTFVSTLSTPTSSLLTTFPATYSFSSS |
| | | #2 | 502-645 | 144 | MTSATTPNVRPTFVSTLSTPTSSLLTTFPATYSFSSSMSASSAGTTHTESISSPPASTSTLHTTAESTLAPTTTTSFTTSTTMEPPSTTAATTGTG |
| | | | | | QTTFTSSTATFPETTTPTPTTDMSTESLTTAMTSPPITSSVTSTNTVT |
| | | #3 | 612-762 | 151 | TTPTPTTDMSTESLTTAMTSPPITSSVTSTNTVTSMTTTTSPPTTTNSFTSLTSMPLSSTPVPSTEVVTSGTINTIPPSILVTTLPTPNASSMTTS |
| | | | | | ETTYPNSPTGPGTNSTTEITYPTTMTETSSTATSLPPTSPLVSTAKTAKTPTTNL |
| | | #4 | 2227-2361 | 135 | TTTETTSHSTPGFTSS ITTTETTSHSTPSFTSS ITTTETTSHDTPSFTSS ITTSETPSHSTPSSTSLITTTKTTSHSTPSFTSS |
| | | | | | ITTTETTSHSAHSFTSS ITTTETTSHNTRSFTSS ITTTETNSHSTTSFTSS |
| MJC4 | E9PDY6 | | 1184-1327 | TQT | PLPVTSPSSASTGHAT PLLVTDTSSASTGHAT PLPVTDASSVSTDHAT SLPVTIPSAASTGHTT |
| | | | | | PLPVTDTSSASTGQATSLLVTDTSSVSTGDTT PLPVTSTSSASTGHVT PLHVTSPSSASTGHAT PLPVTSLSSASTGDTM |
| MUCSAC | P98088 | | 2708-2850 | 142 | TTSAPTT STTSAPTT STISAPTT STTSATTT STTSAPTP RRTSAPTT STISASTT STTSATTT STTSATTTSTISAPTT |
| | | | | | STTLSPTT STTSTTIT STTSAPIS STTSTPQT STTSAPTT STTSGPGT TSSPVPTT STTSAPTT |
| MUC5B | Q9HC84 | #1 | 1889-2029 | 141 | PATSSTATPSSTPGTTWILTKPTTTATTTASTGSTATPTSTLRTAPPPKVLTTTATTPTVTSSKATPSSSPGTATALPALRSTATTPTATSVTPIP |
| | | | | | SSSLGTTWTRLSQTTTPTATMSTATPSSTPETAHTSTVLTATATT |
| | | #2 | 1990-2129 | 140 | TTWTRLSQTTTPTATMSTATPSSTPETAHTSTVLTATATTTGATGSVATPSSTPGTAHTTKVPTTTTTGFTATPSSSPGTALTPPVWISTTTTPTT |
| | | | | | RGSTVTPSSIPGTTHTATVLTTTTTTVATGSMATPSSSTQTSGT |
| | | #4 | 2070-2199 | 130 | ALTPPVWISTTTTPTTRGSTVTPSSIPGTTHTATVLTTTTTTVATGSMATPSSSTQTSGTPPSLTTTATTITATGSTTNPSSTPGTTPIPPVLTTT |
| | | | | | ATTPAATSNTVTPSSALGTTHTPPVPNTMATTHG |
| MUC6 | A0A3Q8T | #1 | 4881-4961 | 123 | TSATSSRLPTPFTTHSPPTGTTPISSTGPVTATSFQTTTTYPTPSHPHTTLPTHVPSFSTSLVTPSTHTVIIPTHTQMATSASIHSMPTGTIPPPT |
| | | | | | TIKATGSTHTAPPMTPTTSGTSQSPS |
| | MU7* | #2 | 4962-5048 | 86 | ASIHSMPTGTIPPPTTIKATGSTHTAPPMTPTTSGTSQSPSSFSTAKTSTSLPYHTSSTHHPEVTPTSTTNITPKHTSTGTRTPVAH |
| MUC7 | Q8TAX7 | | 192-351 | 160 | PPTPSATTQAPPSSSAPPE TTAAPPTPPATTPAPPSSSAPPE TTAAPPTPSATTPAPLSSSAPPE |
| | | | | | TTAVPPTPSATTLDPSSASAPPETTAAPPTPSATTPAPPSSPAPQE TTAAPITTPNSSPTTLAPDTSET |
| | | | | | SAAPTHQTTTSVTTQTTTTKQPTSAP |
| OVGP1 | Q12889 | | 476-564 | 89 | AMTMTSVGHQSMTP GEKALTPVGHQSVTT GQKTLTSVGYQSVTP GEKTLTPVGHQSVTP VSHQSVSPGGTTMTP VHFQTETLRQNTVAP |
| (MUC9) | | | | | |
| MUC13 | Q9H3R2 | | 30-171 | 142 | TTETATSGPTVAAADTTETNFPE TASTTANTPSFPTATS PAPPIISTHSSSTIPT PAPPIISTHSSSTIPI |
| | | | | | PTAADSESTTNVNSLATSDIITASSPNDGLIT MVPSETQSNNEMSPTT EDNQSSGPPTGTALLE TSTLNST |
| MUC17 | Q685J3 | | 2181-2329 | 149 | LSTTPVDTSTPVTNSTEARSSPTTSEGTSMPTSTPSEGSTPFTSMPVSTMPVVTSEAST |
| | | | | | LSATPVDTSTPVTTSTEATSSPTTAEGTSIPTSTLSEGTTPLTSIPVSHTLVANSEVSTLSTTPVDSNTPFTTSTEASSPPPTAEGTSMP |
| MUC19 | Q7Z5P9 | | 3353-3501 | 149 | VTRTTRSSA GLTGKTGLSA GVTGKTGLSA EVTGTTRLSA GVTGTTGPSP GVTGTTGTPA GVTGTTELSA |
| | | | | | GVTGKTGLSSEVTETTGLSY GVKRTIGLSA GSTGTSGQSA GVAGTTTLSA EVTGTTRPSA GVTGTTGLSA EVTEITGISA |
| MUC20 | Q8N307 | | 175-325 | 151 | ESSASSDSPHPVITPSRA SESSASSDGPHPVITPSRA SESSASSDGPHPVITPSRA SESSASSDGPHPVITPSRA |
| | | | | | SESSASSDGPHPVITPSRA SESSASSDGPHPVITPSRA SESSASSDGPHPVITPSRA SESSASSDGPHPVITPSRA |
| MUC21 | Q5SSG8 | | 130-278 | 149 | SGASTATNSDSSTT SSGASTATNSDSSTT SSEASTATNSESSTT SSGASTATNSESSTV SSRASTATNSESSTT |
| | | | | | SSGASTATNSESRTT SNGAGTATNSESSTT SSGASTATNSESSTP SSGAGTATNSESSTT SSGAGTATNSESSTV |
| MUC22 | E2RYF6 | | 336-483 | 148 | GTTTASMAG SETTVSTAG SETTTVSITG TETTMVSAMG SETTTNSTTS SETTVTSTAG SETTTVSTVG SETTTAYTAD |
| | | | | | SETTAASTTG SEMTTVFTAG SETITPSTAG SETTTVSTAG SETTTVSTTG SETTTASTAH SETTAASTMG |
| GP1BA | P07359 | | 281-499 | 219 | PTLGDEGDTDLYDYYPEEDTEGDKVRATRTVVKFPTKAHTTPWGLFYSWSTASLDSQMPSSLHPTQESTKEQTTFPPRWTPNFTLHMESIT |
| | | | | | FSKTPKSTTEPTP SPTTSEPVPEPAP NMTTLEPTP SPTTPEPTSEPAP |
| | | | | | SPTTPE PTSEPAP SPTTPEPTSEPAP SPTTPEPTPIPTI ATSPTILVSATSLITPKSTFLTTTKPVSLLESTKKTIPELD |
| Ctrl | | | | 67 | PAEAAATPAPAK AEAAATPAPAK AEAAATPAPAK AEAAATPAPAK AEAAATPAPAK AEAAATPAPAK |

*Uniprot ID has been reported in Frida Svensson et al. (2018) Scientific reports 8, 17503.
**The amino acid number is derived from the incomplete version of human MUC2 sequence registrated in Uniprot Q02817

### EXAMPLES

The purpose of the following examples is given as an illustration of various aspects of the invention and are thus not meant to limit the present invention in any way. Along with the present examples the methods described herein are presently representative of preferred aspects, are exemplary, and are not intended as limitations on the scope of the invention. Changes therein and other uses which are encompassed within the spirit of the invention as defined by the scope of the claims will occur to those skilled in the art.

Example 1—Engineering Strategy for Display of the Human Mucinome

For long mucins have represented a black box in exploring the molecular cues that serve in intrinsic interactions with glycan-binding proteins and in extrinsic interactions with microorganisms⁵³. Dissection of interactions with simple O-glycan structures found on mucins have benefited tremendously from the development of printed glycan arrays^{43,54}, and these have for decades served as essential tools in exploring the interactome of glycans and proteins⁴¹. However, mucins and their large variable TR domains present O-glycans in different densities, and patterns are likely to provide more specific interactions and instructive cues. Mucin TRs differ markedly in sequence, length and numbers within closely related mammals⁹, and this divergence in TRs may have evolved to accommodate specific recognition of higher order patterns and clusters of O-glycans³⁶. We previously provided evidence for this by use of the cell-based glycan array demonstrating that two distinct streptococcal Siglec-like adhesins bind selectively to O-glycans presented on distinct mucin-like domains in O-glycoproteins and mucins³⁶.

Cell-based display strategies allow for presentation of glycans in the natural context of glycoproteins and the cell surface, and this has provided the first experimental evidence for the existence of higher order binding motifs consisting of O-glycans in dense patterns³⁶. We hypothesized that the main cues for the microbiota lie in the TR regions that display O-glycans of diverse structures and position in unique patterns. The traditional interpretation is that the divergence in TR sequences has co-evolved with the microbiota to govern refined interactions with larger motifs of O-glycan patterns as recently suggested for streptococcal serine-rich adhesins³⁶. The TR regions of mucins are quite distinct in length and in sequences with distinct spacing of O-glycosites⁵⁵, and TRs in any mucin exhibit individual variability in numbers as well as to some degree in actual sequences¹⁰. Thus, there are rich opportunities for unique codes in mucin TRs, governed by the particular display of patterns and structures of O-glycans. The mucin TRs and their glycocodes may be considered as the informational content of mucins and thus comprise the mucinome. The TR mucinome provides a much greater potential binding epitome than the comparatively limited repertoire of binding epitopes comprised of simple oligosaccharide motifs available in humans⁴².

We therefore sought to capture the molecular cues contained in human mucin TRs and enable molecular dissection of these cues. We developed a cell-based platform for display and production of representative mucin TRs with defined O-glycans. We reasoned that most of the features of human mucin TRs could be displayed in shorter segments of 150-200 amino acids, and used a GFP-tagged expression construct design containing representative TRs from different mucins to produce a library of cell membrane and secreted mucin TR reporters in human embryonic kidney (HEK293) cells with distinct programmed O-glycosylation capacities. Strikingly, we found that these mucin TR reporters could readily be produced as highly homogeneous molecules with essentially complete O-glycan occupancies and with distinct O-glycan structures in amounts that enabled us to characterize the simplest reporters by intact mass spectrometry (MS), and hence circumvent the longstanding obstacles with protease digestion and bottom-up analysis of mucins^{14,15}. We demonstrate that the cell-based mucin display can be used to produce and display defined mucin TRs and mucin-like O-glycodomains with custom designed O-glycosylation.

In FIG. 1 an overview of the concept for the cell-based display and production of human mucin TR reporters with programmed O-glycan structures is presented. The mucin TR reporter expression constructs were designed pairwise for either secretion or cell membrane integration through the inclusion of the C-terminal SEA and transmembrane domain of MUC1, and they all included N-terminal GFP, and FLAG tags³⁶. We generated a comprehensive set of TR reporters containing approximately 200 amino acids from the TR O-glycodomains of most human secreted and membrane bound mucins (FIG. 1). The entire sequences selected as representative for each of the human mucin TR O-glycodomains are shown in FIG. 2 and Table 1, which also illustrates that mucin TRs are imperfect in sequence but presents characteristic patterns of O-glycans. Most of the TR reporters contained multiple TR sequences, but due to the longer sequences of MUC2, MUC3, MUC5B and MUC6 TRs these mucin TR domains had to be covered by several reporters with partly overlapping sequences.

The transmembrane TR reporters were expressed transiently in glycoengineered HEK293 cells that do not appear to express endogenous mucins, and the secreted reporters were expressed stably^{36,56} We took advantage of our previously reported O-glycoengineering strategy to establish designs for homogeneous O-glycosylation capacities that result in attachment of defined O-glycan structures (FIG. 1). The gene engineering of HEK293 cells included designs for O-glycans designated In (KO C1GALT1), STn (KO COSMC/KI ST6GALNAC1), T (KO GCNT1, ST3GAL1/2, ST6GALNAC2/3/4), monosialyl-T (mSTa) (KO GCNT1, ST6GALNACT2/3/4), as well as ST comprised of a mixture of mSTa and disialyl-T (dST) (KO GCNT1)³⁸, and the structures, biosynthetic pathways and genetic regulation are illustrated in FIG. 1. Wildtype HEK293WT cells produce a mixture of mono and disialylated core1 and core2 structures³⁶, and KO of GCNT1 eliminates the core2 structures resulting in a mixture of mono- and disialylated core1 O-glycans (mST and dST). KO of COSMC or C1GALT1 results in complete truncation of O-glycans and the uncapped Tn O-glycan without detectable expression of STn⁵⁷. We engineered capacity for core3 (GlcNAcβ1-3GalNAcα1-O-Ser/Thr) O-glycosylation by using AAVS1 locus targeted KI of the core3 synthase (B3GNT6) on top of KO of COSMC to eliminate competition from the core1 synthase.

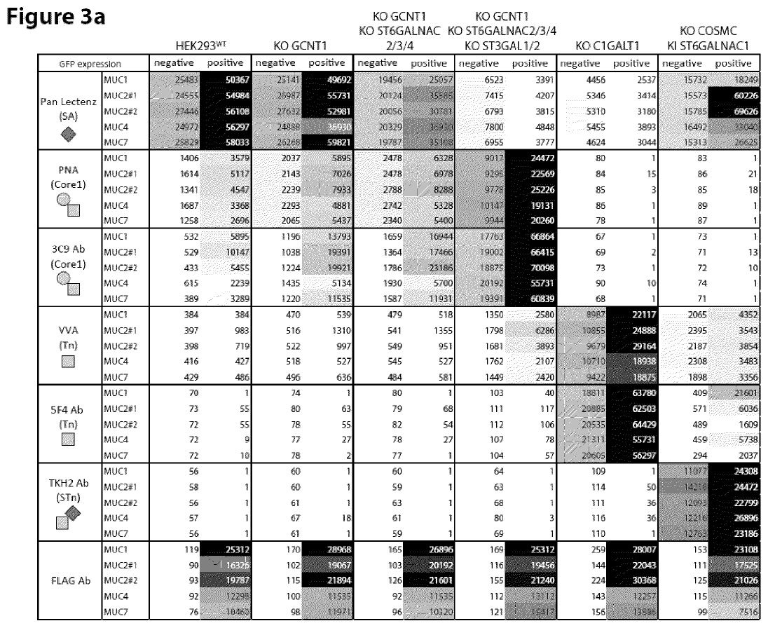

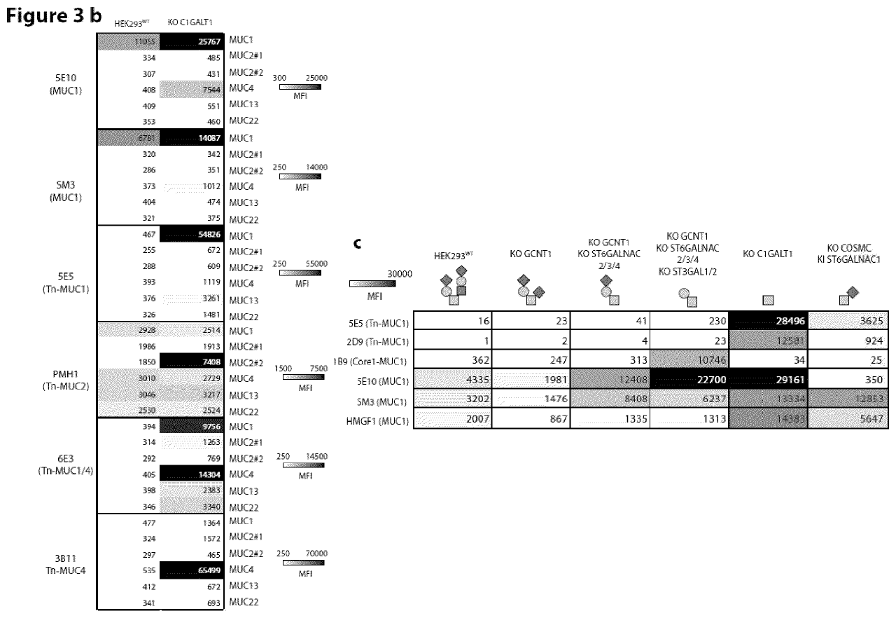

To validate the cell-based mucin TR display platform we first used immunocytology analyses. We previously verified the general glycosylation outcomes of most of the glycoengineering performed in HEK293 cells³⁶. We therefore tested a subset of transiently expressed membrane bound TR reporters with lectins and monoclonal antibodies (mAbs) with well characterized specificities for distinct O-glycan structures (FIG. 3a). There was a substantial window of signal difference in flow cytometry for HEK293 cells with and without expression of the GFP-tagged mucin TR reporter. Thus, the engineered glycosylation capacity for Tn, T, and STn O-glycosylation could be shown both with the cell population not expressing the mucin TRs (GFP-negative) and the transfected cell population expressing these (GFP positive), albeit with higher intensities when mucin TRs were expressed.

We also probed the mucin TR reporters with a panel of mAbs directed to human mucin TR regions, most of which are known to be affected by O-glycosylation either because glycosylation interferes with or blocks binding to the protein core (e.g. mAb to MUC1 such as SM3 or 5E10)⁵⁸ or because O-glycans are required for the binding (e.g. mAbs to Tn-MUC1 (5E5), Tn-MUC2 (PMH1), and Tn-MUC4 (3B11)⁵⁹⁻⁶¹ (FIG. 3b). The observed reactivity patterns were in agreement with the reported specificities of the tested mAbs.

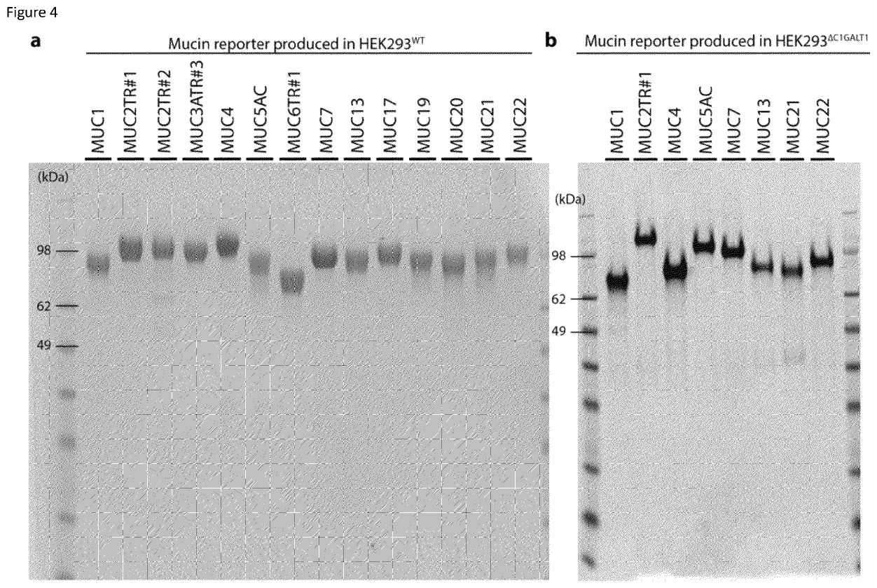

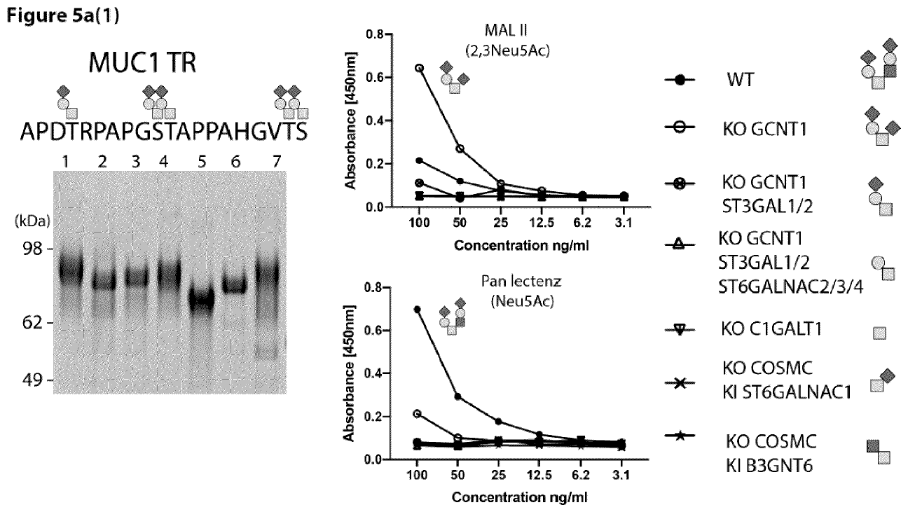

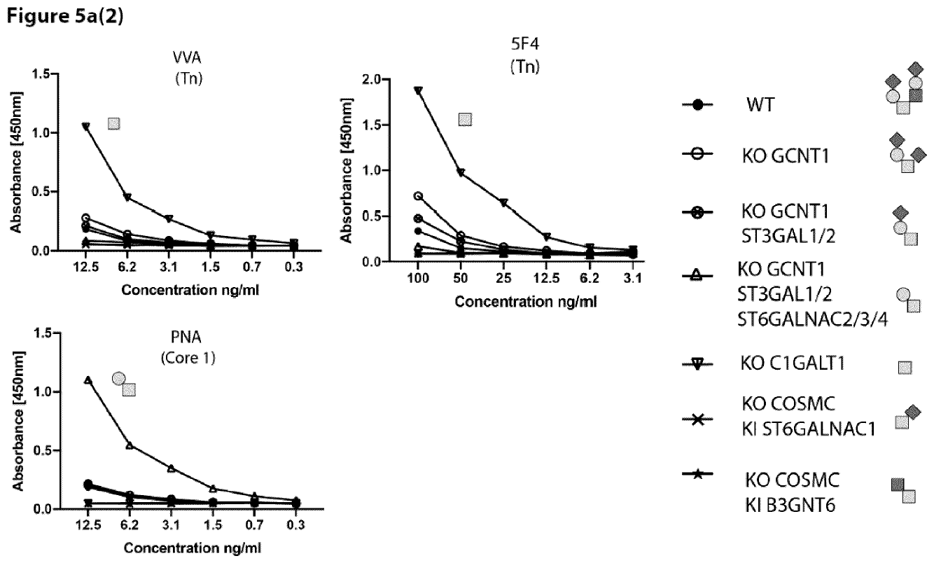

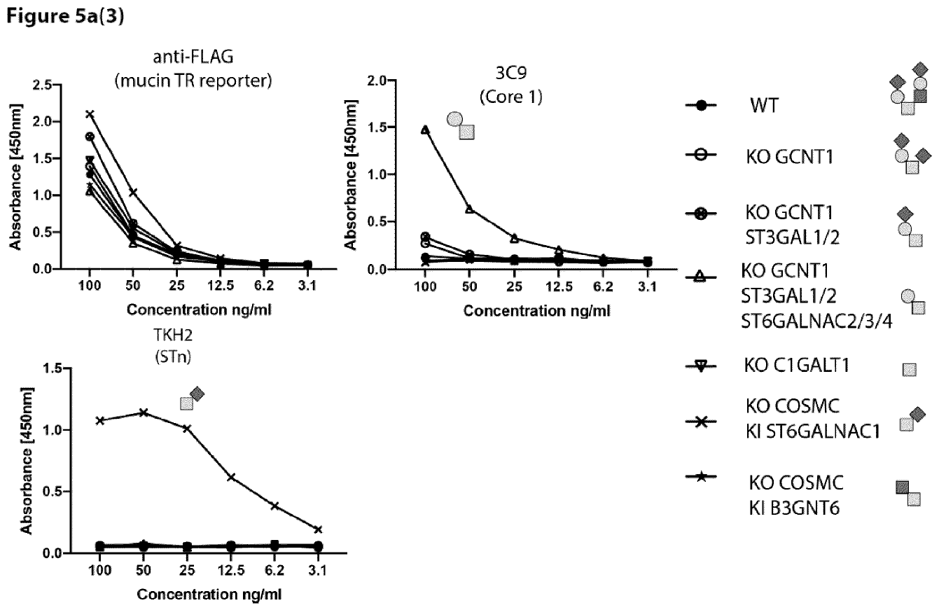

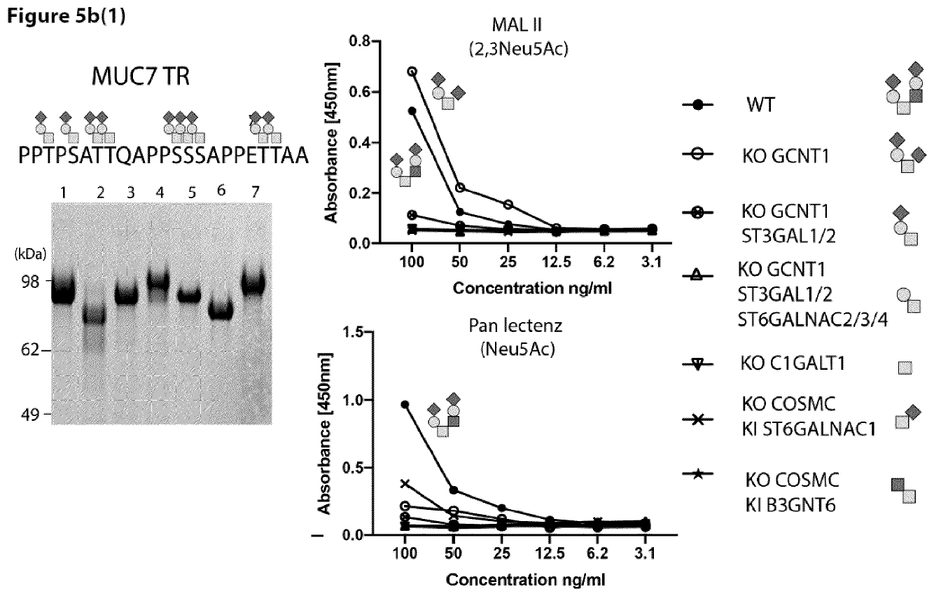

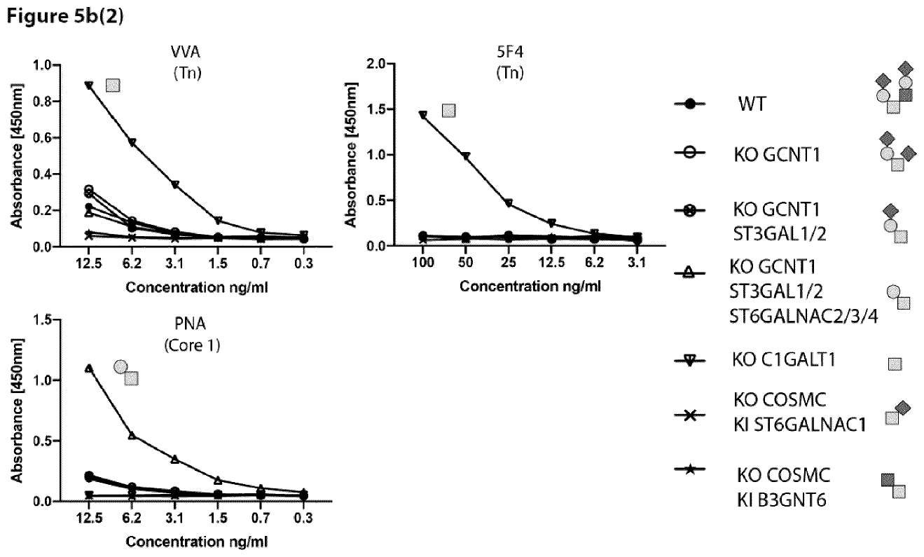

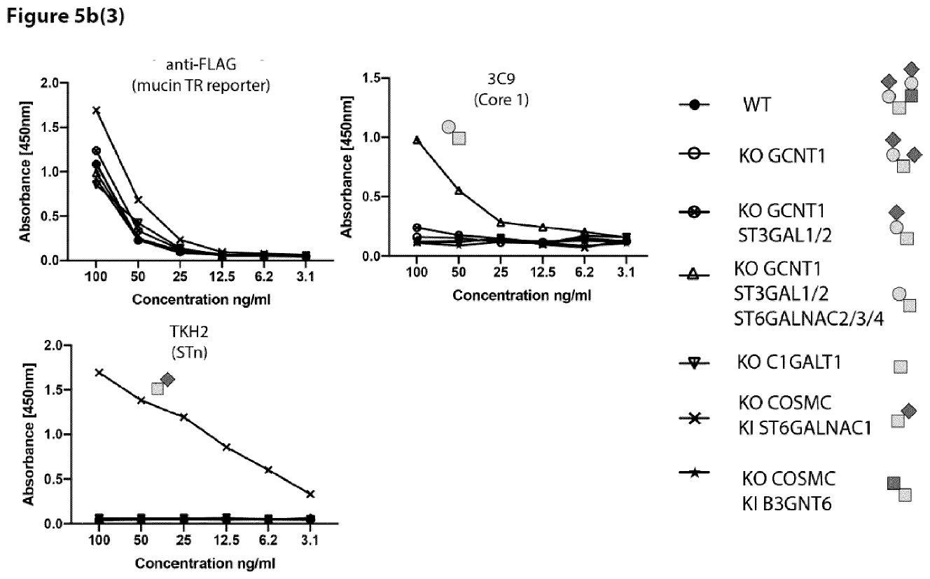

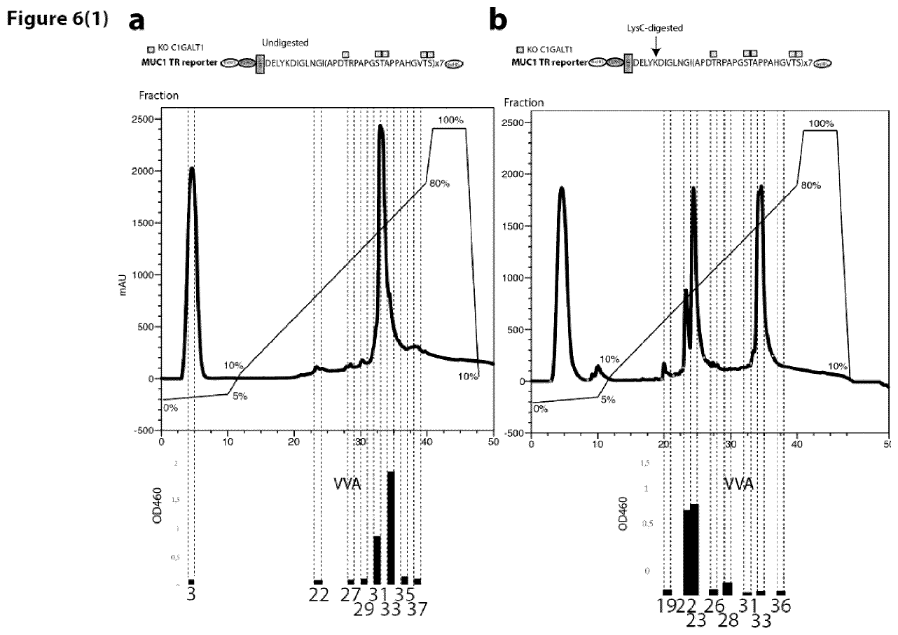

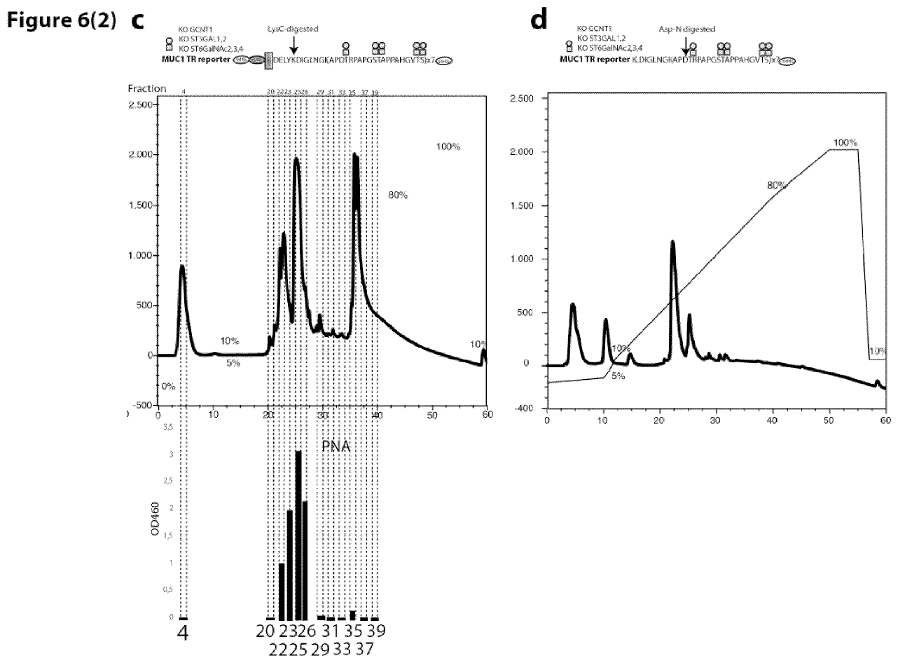

We further performed structural analysis of the isolated secreted mucin TRs. Secreted TR reporters stably expressed in glycoengineered HEK293 cells were isolated by Ni-chromatography and assessed by SDS-PAGE analysis, which showed that the GFP-tagged proteins migrated as distinct rather homogeneous bands (FIGS. 4 and 5). We used LysC digestion to liberate the intact TR O-glycodomains and C4 and C18-HPLC to purify these for further analysis (FIG. 6). For direct intact mass analysis of mucin TRs we used pretreatment with neuraminidase to reduce complexity and facilitate deconvolution and interpretation.

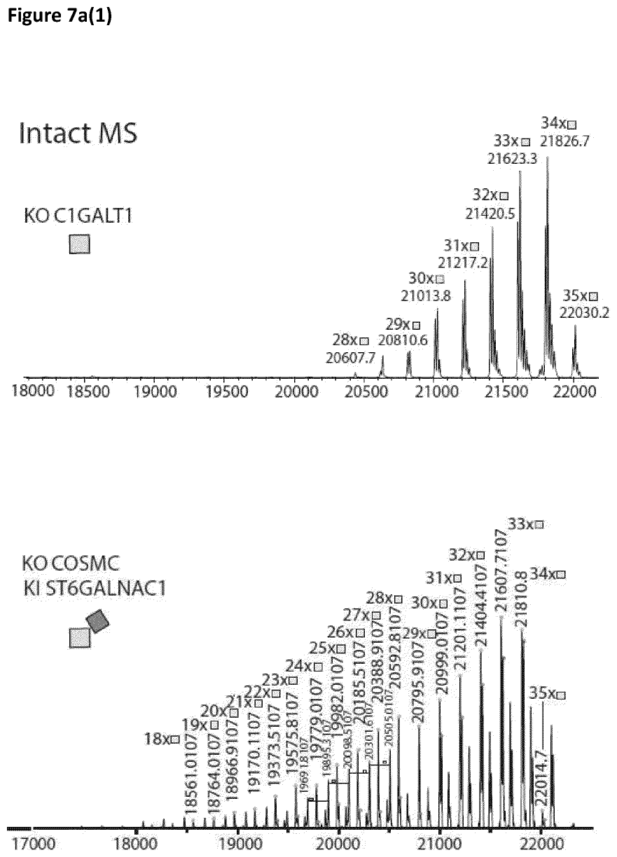

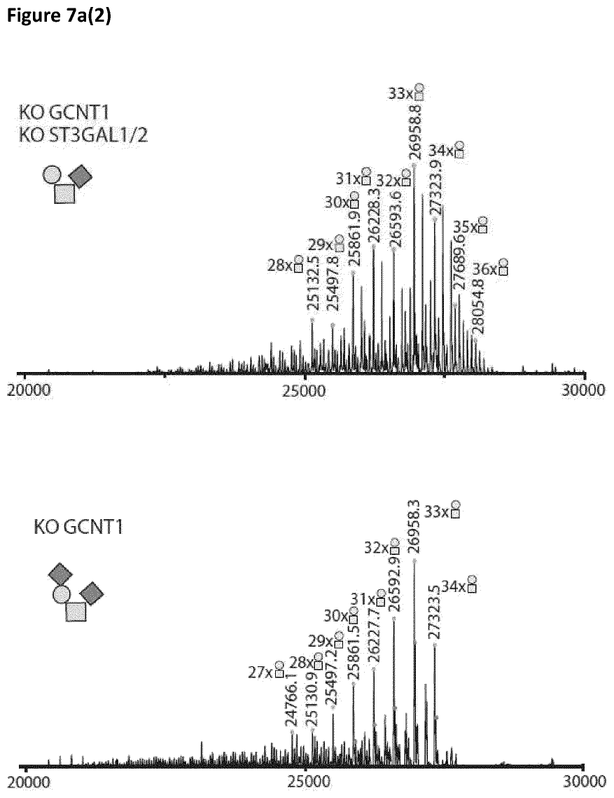

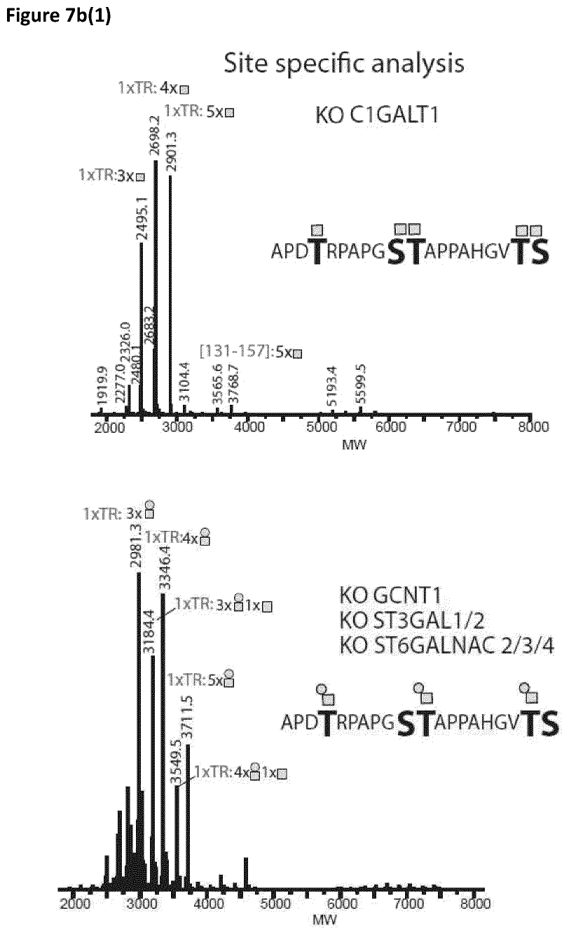

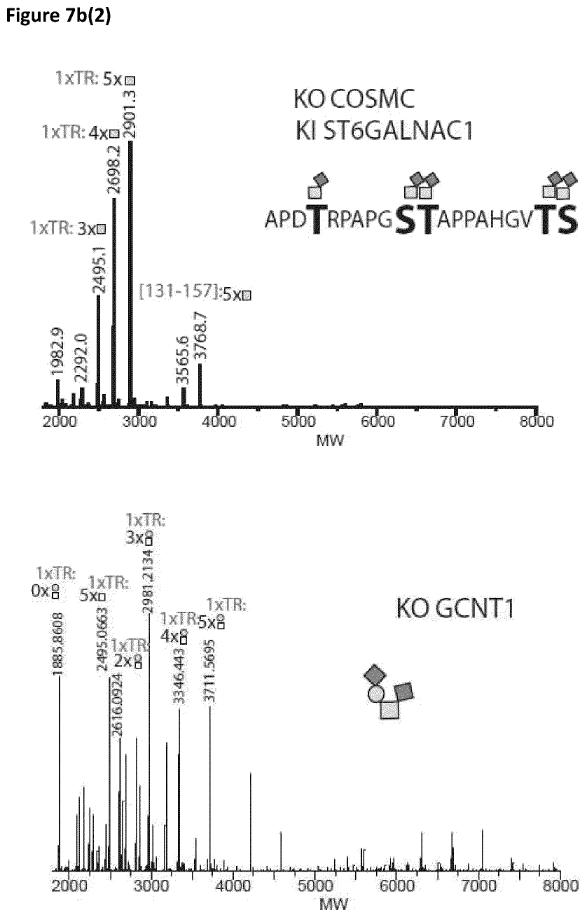

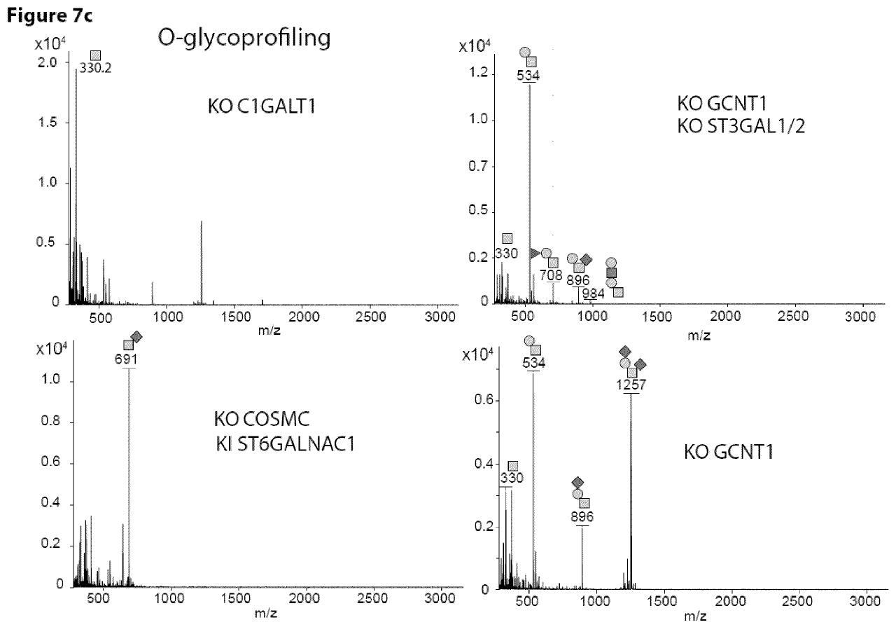

The dense O-glycosylation of mucin TRs in most cases blocks cleavage by peptidases limiting conventional glycoproteomics strategies^{14,16}. However, the MUC1 TRs are cleavable by endoproteinase-Asp-N (AspN) in the PDTR sequence^{17, 18,19} and we therefore used the MUC1 reporter for full characterization (FIG. 7). The MUC1 reporter contains³⁴ predicted O-glycosites and includes six 20-mer TRs and a C-terminal TR where the last GVTSA sequence proceeds into the 6XHis tag. We used LysC to cleave the purified GFP-tagged reporter and isolate the TR O-glycodomain for LC-MS intact MS analysis (FIG. 7a). The simplest Tn glycoform (HEK293^{KO C1GALT1}) revealed a rather small range of incremental masses corresponding to HexNAc (203.08) centered around the predicted protein size (m/z 14,902.14) with 28-35 HexNAc residues, while the T (HEK293^{KO GCNT1, ST3GAL1/2, ST6GALNAC2/3/4}) and ST (HEK293^{KO GCNT1}) glycoforms after neuraminidase treatment generated the same narrow range of predicted 28-35 Hex-HexNAc disaccharides. In contrast, the STn glycoform (HEK293^{KO CIGALT1 KI ST6GALNAC1}) analyzed after treatment with neuraminidase produced a slightly broader range of detectable glycoforms from 18-35 HexNAcs, suggesting that ST6GALNAC1 competes partly with the completion of GalNAc glycosylation by GALNTs and in agreement with previous studies^{62,63}. Analysis of the MUC1 TR reporters after AspN digestion revealed that the predominant 20-mer glycopeptides derived from Tn-MUC1 and STn-MUC1 were those with 4-5 O-glycans per TR (FIG. 7b). For the bottom up analysis we also had to use pretreatment with neuraminidase because the sialylated glycoforms were poorly digested by AspN. For the MUC1 reporters with higher glycan complexity (T-MUC1 and ST-MUC1) the most abundant glycopeptide variants appeared to be shifted towards 3-4 O-glycans per TR (FIG. 7b), however, this result may be biased by inefficient AspN digestion since the intact MS analysis did not show the same tendency (FIG. 7a). Finally, we confirmed the glycoengineering by O-glycan profiling of released O-glycans from the MUC1 TR reporters with five different O-glycan designs by MALDI-TOF analysis (FIG. 7c).

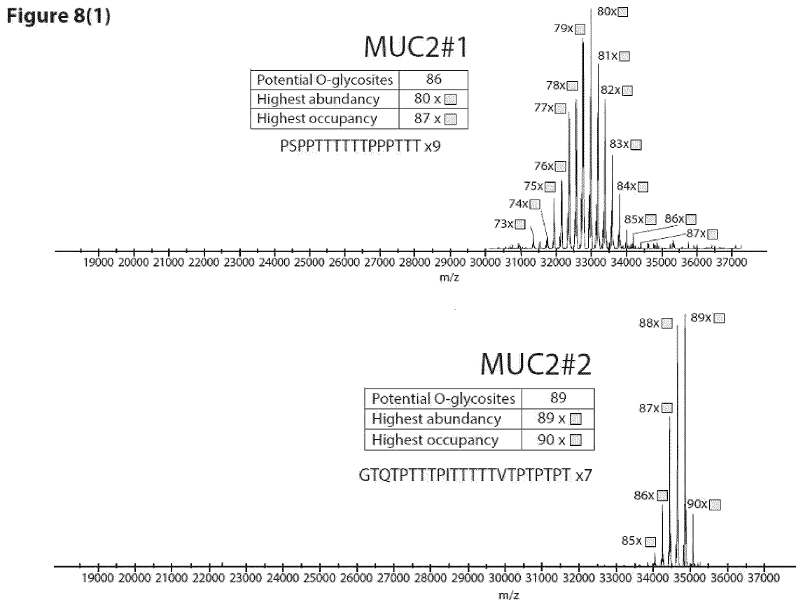

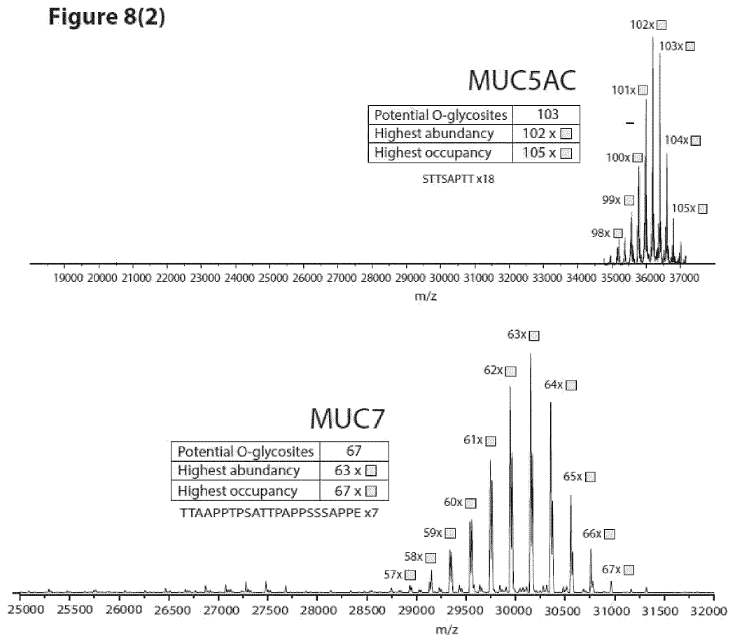

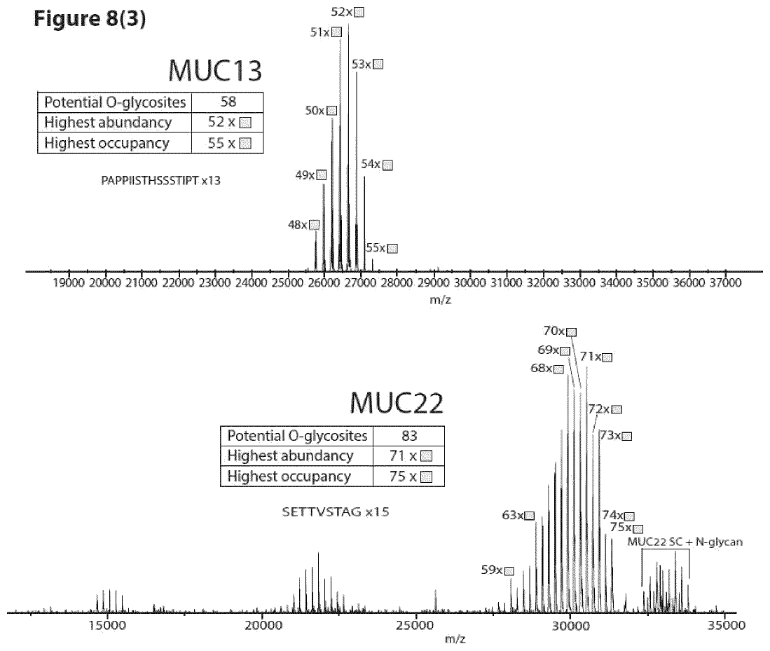

The promising results obtained with intact MS analysis of the MUC1 TR glycodomains prompted intact MS analysis of the simplest Tn glycoforms of MUC2, MUC5AC, MUC7, MUC13, and MUC21 TR glycodomains, which showed similarly high occupancy of available glycosites with rather homogeneous patterns (FIG. 8). For most mucin TRs the proteoform with the highest number of HexNAc residues correlated with the number of potential O-glycosites with the most abundant proteofoms centered close to or a little lower than this. However, for the MUC21 TR reporter the highest abundant peaks were centered around 68-71 with 83 potential O-glycosites suggesting lower occupancy.

Example 2—Applying the Cell-based Mucin Array for Analysis of the Cleavage Activity of the Glycoprotease StcE

Cell-based display strategies allow for presentation of glycans in the natural context of glycoproteins and the cell surface, and this has provided the first experimental evidence for the existence of higher order binding motifs consisting of O-glycans in dense patterns 36. We hypothesized that the main cues for the microbiota lie in the TR regions that display O-glycans of diverse structures and position in unique patterns. The traditional interpretation is that the divergence in TR sequences has co-evolved with the microbiota to govern refined interactions with larger motifs of O-glycan patterns as recently suggested for streptococcal serine-rich adhesins³⁶. The TR regions of mucins are quite distinct in length and in sequences with distinct spacing of O-glycosites⁵⁵, and TRs in any mucin exhibit individual variability in numbers as well as to some degree in actual sequences¹⁰. Thus, there are rich opportunities for unique codes in mucin TRs, governed by the particular display of patterns and structures of O-glycans. The mucin TRs and their glycocodes may be considered the informational content of mucins and thus comprise the mucinome. The TR mucinome provides a much greater potential binding epitome than the comparatively limited repertoire of binding epitopes comprised of simple oligosaccharide motifs available in humans⁴².

We therefore sought to capture the molecular cues contained in human mucin TRs and enable molecular dissection of these cues. We developed a cell-based platform for display and production of representative mucin TRs with defined O-glycans. We reasoned that most of the features of human mucin TRs could be displayed in shorter segments of 150-200 amino acids, and used a GFP-tagged expression construct design containing representative TRs from different mucins to produce a library of cell membrane and secreted mucin TR reporters in human embryonic kidney (HEK293) cells with distinct programmed O-glycosylation capacities. Strikingly, we found that these mucin TR reporters could readily be produced as highly homogeneous molecules with essentially complete O-glycan occupancies and with distinct O-glycan structures in amounts that enabled us to characterize the simplest reporters by intact mass spectrometry (MS), and hence circumvent the longstanding obstacles with protease digestion and bottom-up analysis of mucins^{14,20}. We demonstrate that the cell-based mucin display can be used to produce and display defined mucin TRs and mucin-like O-glycodomains with custom designed O-glycosylation.

Availability of the cell-based mucin display platform enabled for the first time detailed analysis of the substrate specificities of microbial glycopeptidases such as the StcE glycoprotease. Moreover, using this platform we were able to demonstrate that StcE cleaved several different types of mucins, but not MUC1 as previously indicated²³. Moreover, we could demonstrate that StcE efficiently cleaves mucin TRs with different types of O-glycans including core1 and core2 O-glycans, but importantly StcE cleavage is blocked by core3 and sialyl-Tn O-glycans found predominantly in the human intestine.

The mucin display platform is ideal for discovery and exploration of mucin degrading enzymes such as the pathogenic glycoprotease StcE²¹⁻²⁴. EHEC is a food-derived human pathogen able to colonize the colon and cause gastroenteritis and bloody diarrhea. Strains of the O157:H7 serotype carry a large virulence plasmid pO157:H7 that directs secretion of StcE^{21,26}. StcE is predicted to provide EHEC with adherence to the gastrointestinal tract and ability to penetrate through the mucin layers via its impressive mucin degrading properties⁶⁴. StcE cleaves the C1 esterase inhibitor glycoprotein (C1-INH) that contains a highly O-glycosylated mucin-like domain and is required for complement activation²¹. StcE was previously shown to cleave several mucins including MUC1, MUC7 and MUC16^{22,23,25,65}, and the cleavage required O-glycosylation and accommodated complex O-glycan structures²³. The gut microbiome is contained in a network of the gel forming mucin MUC2 that forms the loose outer mucin layer, and a dense inner layer of MUC2 forms a barrier and prevents the microbiota to reach the underlying colonic epithelium^{66,67}. Cleavage of the MUC2 mucin layers by StcE would destroy the important barrier function.

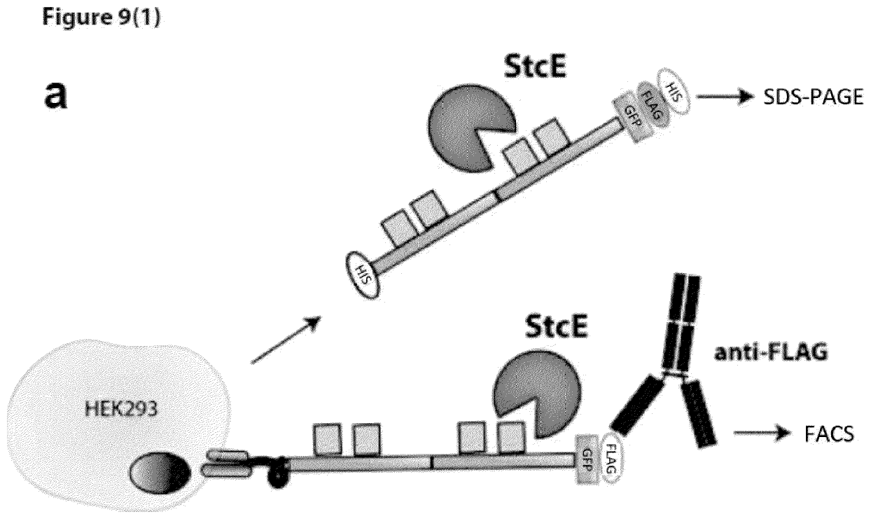

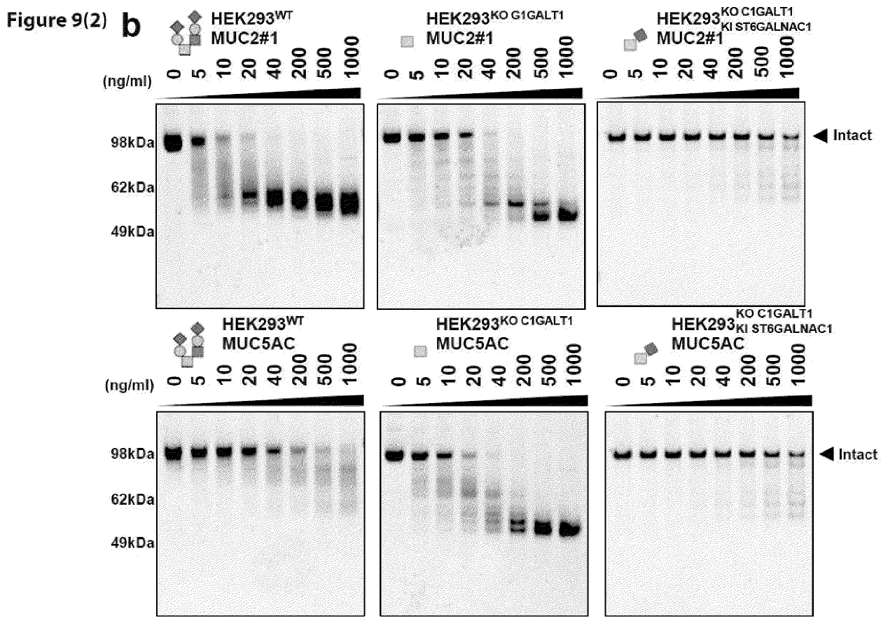

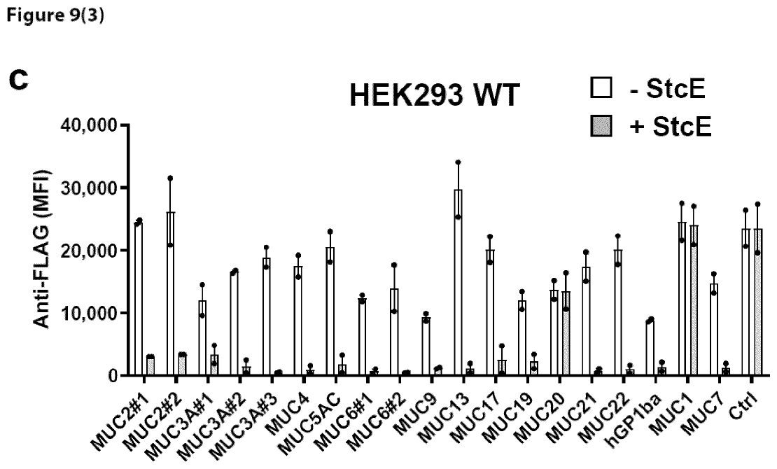

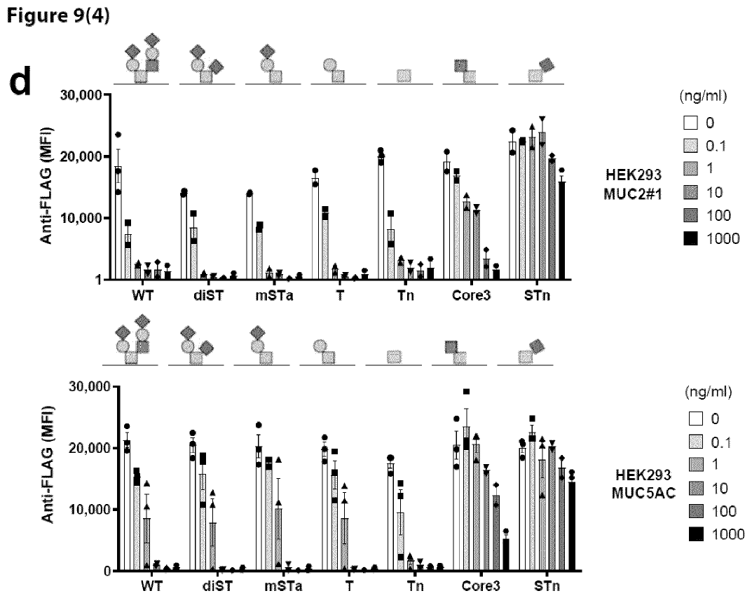

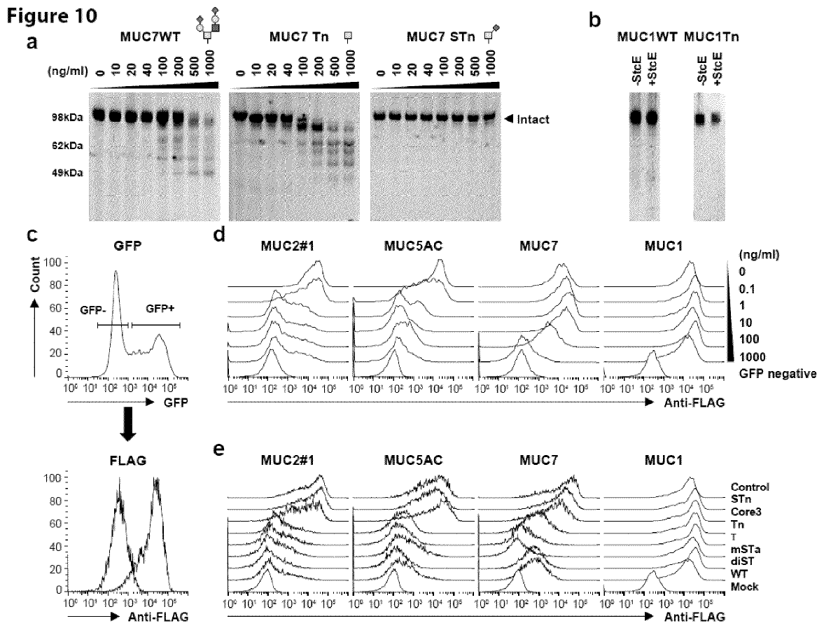

We used purified TR reporters and those displayed on cells to further explore the fine substrate specificity of recombinant purified StcE glycoprotease and to dissect its reported mucin-binding properties (FIG. 9). First, we found that StcE efficiently cleaved isolated MUC2 and MUC5AC reporters with core2 (HEK293^{WT}) and Tn O-glycans already at low concentrations of 10-40 ng/ml (1:2,500-500, StcE : TR reporter ratio), while the STn glycoform was essentially resistant to cleavage (FIG. 9b and FIG. 10a). Next, we explored the selectivity of StcE with the 20 membrane bound mucin TRs displayed on HEK293^{WT} cells by monitoring loss of the N-terminal FLAG tag by flow cytometry using fluorescent anti-FLAG tag antibodies (FIG. 9a). StcE efficiently cleaved most of the mucin TRs with the notable exception of MUC1 and MUC20, as well as the control TR reporter designed with a single O-glycosite (FIG. 9c and FIGS. 10c, d). Dose-titration analysis in both assays showed low ng/ml cleavage for most mucin TR reporters, while no cleavage of the MUC1 TR reporter was found even at 10 μg/ml (FIGS. 10b, d). StcE was shown previously to cleave the entire MUC1 expressed on cancer cells, but this may be due to cleavage outside the TR region as the proposed StcE cleavage motif (S/T-X-S/T) is absent from the well conserved TRs^{23,27}. Finally, we dissected the effect of O-glycan structures on StcE cleavage using the MUC2 and MUC5AC TR reporters, and found that core1 and core2 O-glycans including Tn are efficiently cleaved, while STn as well as core3 O-glycans efficiently blocked proteolysis (FIG. 9d and FIG. 10e).

The cell-based mucin display platform presented here offers a unique resource with wide applications and opportunities for discovery and dissection of molecular properties of natural human mucins and other glycoproteins with mucin-domains. The informational cues harbored in mucin TRs with their distinct patterns and structures of O-glycans can be addressed with well-defined molecules in a variety of assay formats. This was illustrated by our use of the mucin display to dissect the fine substrate specificity of the mucin-destroying glycoprotease StcE derived from pathogenic EHEC^{21,22}, demonstrating clear selectivity for both distinct mucin TRs and O-glycoforms, and importantly discovering that the normal core3 O-glycosylation pathway in colon actually inhibits StcE digestion of MUC2.

Example 3—Analysis of the Tissue Binding Properties of the Glycoprotease StcE

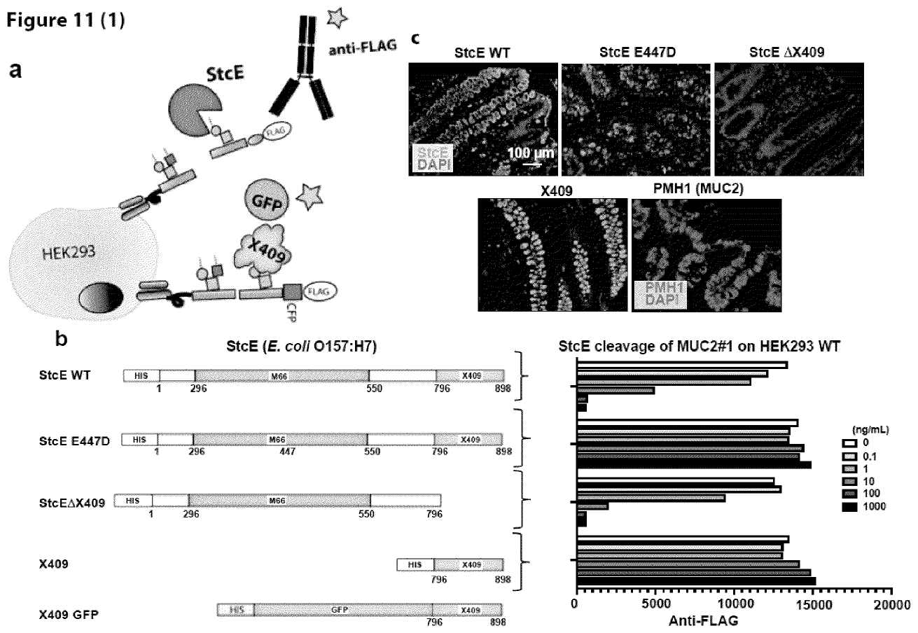

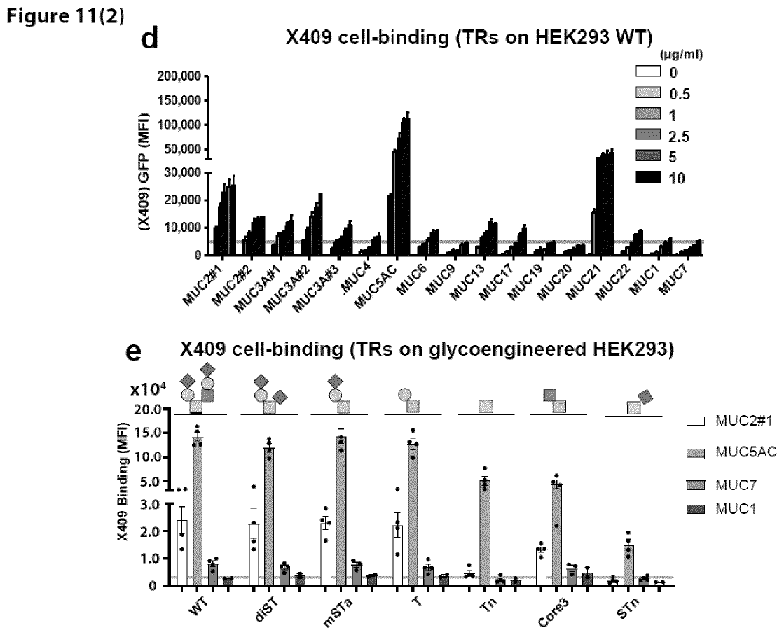

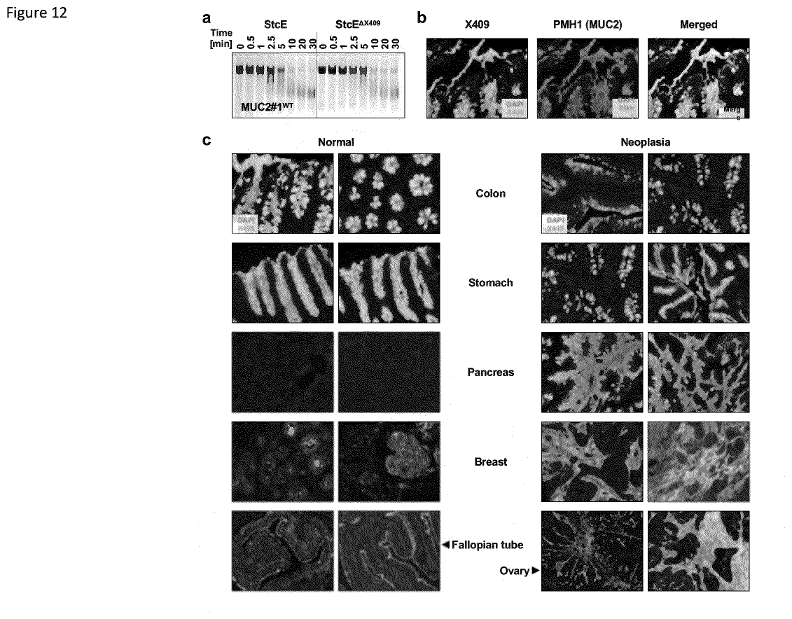

By close examination of the 3-D structure of StcE²⁴ we noticed that the protein contained a C-terminal domain opposite to the catalytic metalloprotease domain (M66), and we hypothesized that this could have a function for StcE. The small domain is a peptide of approximately 100 amino acids, and by detailed sequence analysis we predicted that this could represent an evolutionarily mobile binding module (here designated X409). To test the potential function of the X409 module we first analyzed if deleting this domain affected the mucin cleaving function of StcE using the cell-based mucin display platform. Surprisingly, we found that StcE without X409 retained its remarkable ability to cleave mucin TRs. We therefore next considered whether the module had a role in the mucin binding properties of StcE, which was previously demonstrated with the catalytically inactivated StcE^{E447D} mutant27. We first tested the wildtype StcE active enzyme in binding to human mucosal tissues, and surprisingly found that wildtype StcE without the inactivating mutation bound mucin producing cells similar to the StcE^{E447D} mutant (FIG. 11c). This suggested that the original proposal that the catalytic unit when inactivated could serve as a substrate binding protein was incorrect²⁷. We then tested the active StcE enzyme without the X409 module and discovered complete loss of binding (FIG. 11c). This suggested that the X409 module functions as a mucin-binding module, and analysis of a fusion protein of the X409 module (GFP fusion with FLAG and HIS tags) surprisingly showed that this module alone exhibited strong binding to mucin producing cells in the gastric and colonic mucosa (FIGS. 11c and 12b ,c). We therefore studied the mucin binding properties of the X409 module with the mucin TR display platform and discovered remarkably selective mucin binding properties for distinct mucins (e.g. MUC5AC, MUC2, MUC21) but no binding to several other mucins (e.g. MUC1) (FIG. 11d). These results are strikingly different from those reported previously at many levels, and it is currently not possible to reconcile the reasons for these contradictions. However, the use of well-defined mucin TR reporters through availability of the cell-based mucin TR display platform, and analysis of the isolated X409 module without additional binding properties provided by the catalytic unit with or without inactivation may in part play a role.

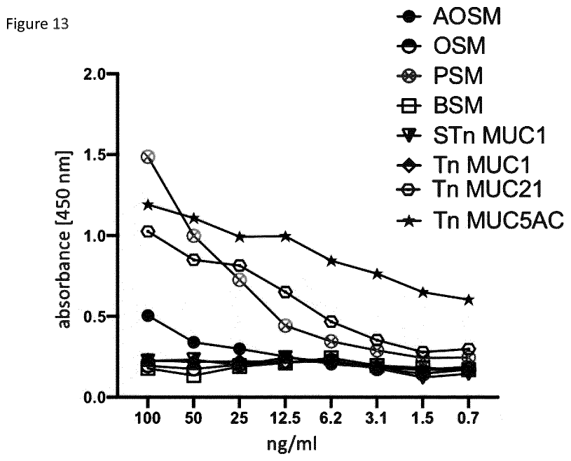

Detailed analysis of the binding pattern with the mucin TR display suggested that a common feature for X409 binding was a sequence motif of 5-6 clustered O-glycans. To test this hypothesis not only by the cell-based human mucin TRs, we tested isolated commercial animal mucins (BSM, PSM, and OSM) by ELISA. Only PSM is known to contain such long clusters of O-glycans, and X409 exhibited binding only to PSM and not to BSM and asialo OSM (FIG. 13).

The small X409 peptide module offers a unique molecule for binding to select mucins. We show that a fusion protein comprising X409 can be used to bind gastric and intestinal mucosa, and this offers an elegant way to deliver and retain molecules at such anatomical sites for diagnostic and therapeutic purposes.

StcE plays a role in adherence of EHEC to the intestinal epithelium by binding mucins^{22,24,28}, and a catalytically inactive mutant of StcE (StcE^{E447D}) exhibits broad binding to mucin producing cells in tissue sections^{27,24}. Examination of the 3-D structure of StcE revealed that the protein contained a C-terminal domain (here designated X409) opposite to the catalytic metalloprotease domain (M66). The X409 module was predicted to be important for the catalytic function of StcE, and we therefore produced a StcE mutant construct without this domain (StcE^{ΔX409}) (FIG. 11b and FIG. 12a). Surprisingly, the StcE^{ΔX409} mutant exhibited unaltered cleavage activity with the MUC2 TR reporter expressed in HEK293^{WT} cells. We then produced a GFP-X409 fusion protein and HIS-tagged X409 and found that these did not have enzymatic activity (FIG. 11b and FIG. 12a). We postulated that therefore, the X409 could potentially represent an evolutionarily mobile binding module. To test the role of the X409 module for the tissue binding properties of StcE we used the StcE^{ΔX409} mutant, and surprisingly found that deletion of X409 completely abrogated the binding properties of StcE observed with both the WT and the E447D mutant (FIG. 11c). Note that the binding of active StcE, inactive StcE^{E447D}, as well as X409 alone fully recapitulated the tissue binding found previously with the inactive StcE^{E447D} mutant^{23,25-27,68}. Interestingly, the StcE/X409 staining appeared to overlap with MUC2 expression in human colon as shown by co-localization of staining with a mAb directed against Tn-MUC2 (PMH1) (FIG. 12b)1. The X409 module displayed strong binding to normal human colon and stomach tissues and colon and stomach cancers. While we found no or low binding to other normal tissues including pancreas and breast, strong binding to the counterpart cancer tissues was observed (FIG. 12c). To further explore the binding properties of the X409 module we tested the cell-based mucin TR display, and this revealed remarkable selective binding to the human mucin MUC5AC and MUC21 TRs as well as MUC2 and MUC3 when these carried core2 O-glycans (FIG. 11d). Importantly, many mucin TRs including MUC1 did not bind (FIG. 11d), showing highly selective binding to gastrointestinal mucins.

We further tested the affect O-glycan structures on the mucin TRs have on the binding of X409, and surprisingly the strong binding to MUC2 and MUC5AC was only slightly influenced by the O-glycan structures attached to the TRs, although weaker binding to TRs carrying Tn, core3 and especially STn O-glycans was observed (FIG. 11e). These results clearly demonstrate that the X409 module mediates the mucin-binding properties of StcE in striking contrast to previous reports²⁷. However, it is also important to recognize that the catalytic unit of StcE may mediate selective binding to dST O-glycans as previously reported²⁴, and this may obscure the binding results obtained previously with StcE^{E447D}. Interestingly, a recent report described use of the full StcE^{E447D} mutant for affinity isolation of O-glycoproteins, and found that the mutant enzyme enriches not only mucins but also a variety of other O-glycoproteins³⁰. Given that StcE, StcE^{E447D} and X409 alone exhibited the same highly selective tissue binding to mucin producing cells, it is likely that the dST glycan binding properties of the catalytic unit of StcE, previously found with high density of glycans printed on glass slides²⁴, do not contribute substantially to binding to tissues as the mucin producing cells in the normal tissues tested mainly produce more complex core2 and core3 O-glycans. Core3 O-glycosylation is restricted to the gastrointestinal tract in human, and in the mouse MUC2 is mainly glycosylated with core1 and core2 O-glycans that are also commonly found outside the gastrointestinal tract in humans^{12,69,70}. In cancer cells the O-glycosylation process is often altered and cancer cells may predominantly produce core1 O-glycans that may bind the catalytic unit of StcE.

The unique binding properties of X409 to select mucins independent of the structure of the attached O-glycans (FIGS. 11d,e), is very different from the binding properties found with lectins that primarily bind terminal glycan structures, although some lectins do bind internal motifs in larger glycan structures as in the case of lectins binding to the mannose core of N-glycans⁷¹. Examining the mucin TR sequences that show selective binding to (MUC5AC, MUC5B and MUC21 as well as with lower intensity MUC2 and MUC3) (FIG. 2), revealed one common distinguishing feature being the presence of clusters of 5-7 (S/Tₙ₌₅₋₇) O-glycan motifs. Since the O-glycan structures are not essential for binding, but necessary since X409 does not bind unglycosylated mucin TRs, it is likely that clusters of 5-7 O-glycosites on a mucin peptide compose a unique form of binding site for X409. Longer clusters of such O-glycans are rarely found in human proteins and largely limited to the few identified human mucin TRs studied here⁹. X409 therefore represent a novel class of binding modules that recognize select mucin TRs and require specific peptide sequences and the presence of multiple O-glycans. To test this hypothesis further we used natural isolated animal mucins that are known to contain large O-glycan clusters (porcine submaxillary mucin, PSM), and known not to contain such (bovine submaxillary BSM and ovine submaxillary OSM), and tested binding of X409 by ELISA (FIG. 13).

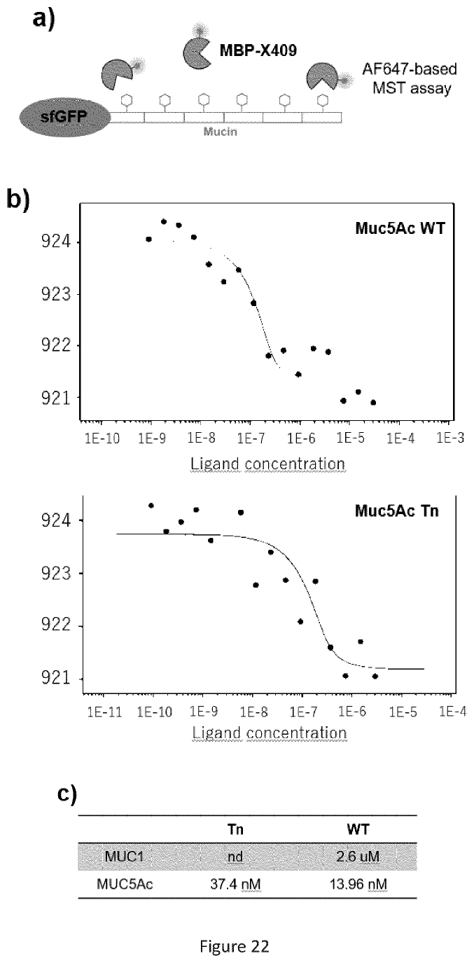

The binding affinity of traditional lectins to glycans is typically weak (K_{d}=10⁻³-10⁻⁶ M, mM to μM) in comparison to binding affinity of antibody-antigen interactions that can be <10⁻⁹ M (<nM), although increased binding affinity of lectins to glycans may be observed with multivalent interactions. The unique mucin-binding properties of X409 with select binding to distinct mucins and without select binding to distinct O-glycan structures prompted testing of the binding affinity and mode of binding to a mucin reporter with different defined O-glycans attached (FIG. 22). We chose the MUC1 and MUC5Ac reporters and two glycoforms including the WT glycoform comprised of core2 sialylated O-glycans and the most simple Tn O-glycans. Flow cytometry analysis of X409 binding to cells displaying these mucin reporters showed high binding to MUC5Ac and very low binding to MUC1, and higher binding to WT MUC5Ac than to Tn MUC5Ac (FIG. 11e). Using a microscale thermophoresis (MST) assay with AlexaFluoro647 labeled MBP-X409 and the secreted purified reporters, extremely high affinity binding of X409 to WT MUC5Ac (14 nM) and lower binding to Tn MUC5Ac (37 nM) (FIGS. 22b,c) was demonstrated. In contrast, the binding affinity to MUC1 with the WT glycoform was estimated to be >1,000× lower (2.6 μM) and not measurable for Tn-MUC1. Binding affinity was determined by Monolith MicroScale Thermophoresis (MST) from Nanotemper. Monolith uses MST technology to quantify molecular interactions between a target and ligand by detecting changes in fluorescence intensity while a temperature gradient is applied over time. Mucin TR reporters and the X409 module were fluorescently tagged (GFP) according to the manufacturers protocol, and the binding affinity was automatically determined at the end of each run. The affinity constant (K_{d}) was calculated from a fitted curve plotting normalized fluorescence against concentration of ligand. Analysis of the binding by mass photometry revealed that one molecule of X409 bound to one mucin molecule and confirmed the high affinity binding.

The high affinity binding properties of X409 to select mucins with elaborated mature O-glycans is unique and dissimilar to traditional lectins and glycan-binding proteins with low affinity. Moreover, the select binding to distinct mucins and not for example MUC1 strongly indicate that X409 has unique binding properties and recognize a motif comprised of the innermost part of multiple O-glycans attached to the mucin protein backbone. A similar high affinity binding to glycosylated mucins have only been described for monoclonal antibodies to the cancer-associated Tn glycoform of MUC1 (Tn-MUC1), which recognize the glycans and part of the protein backbone. Thus, the X409 mucin-binding module represent a new class of non-immunoglobulin binders that have selectivity for distinct glycoproteins and high affinity binding like antibodies while relying on glycans for binding similar to lectins.

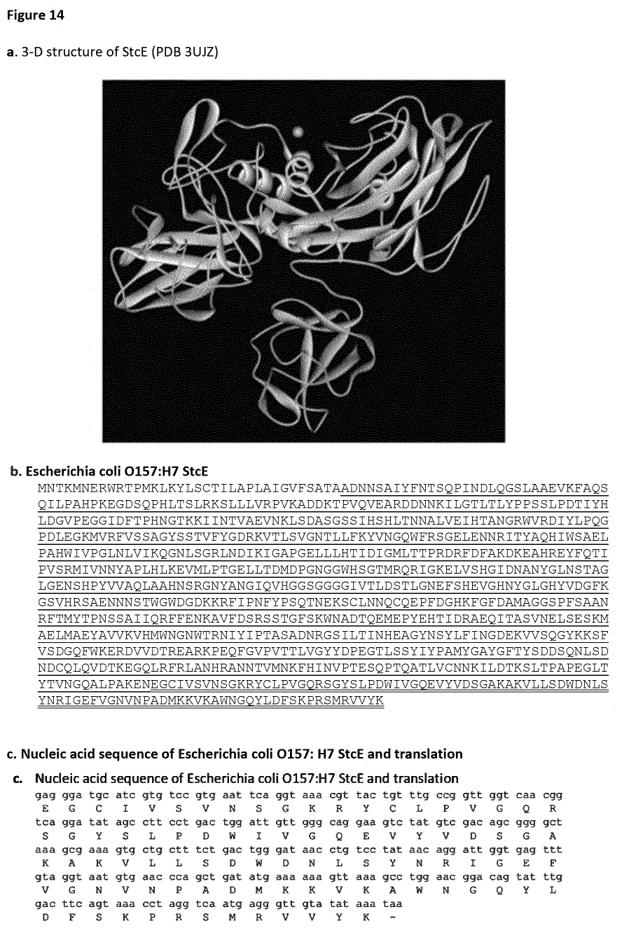

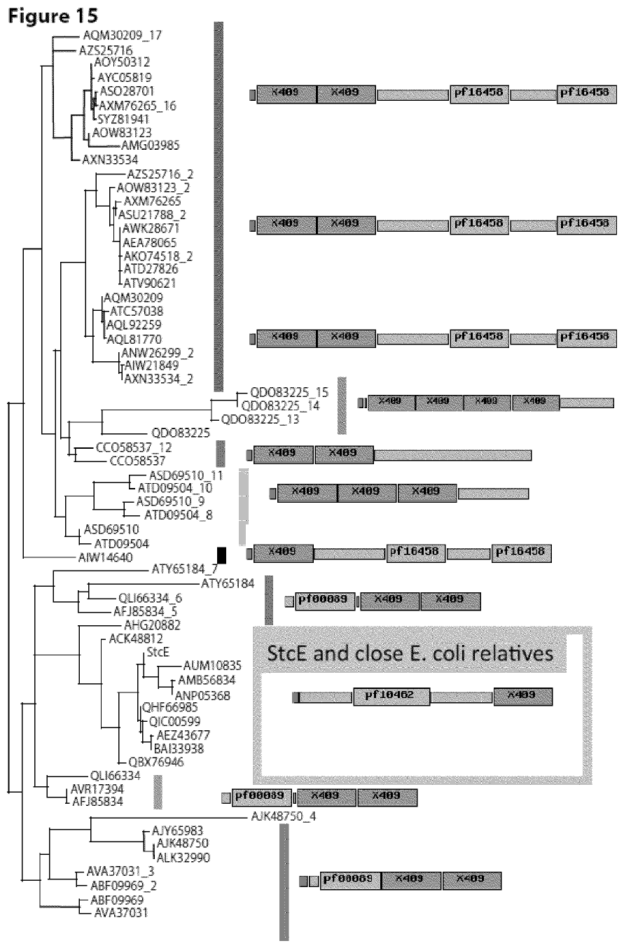

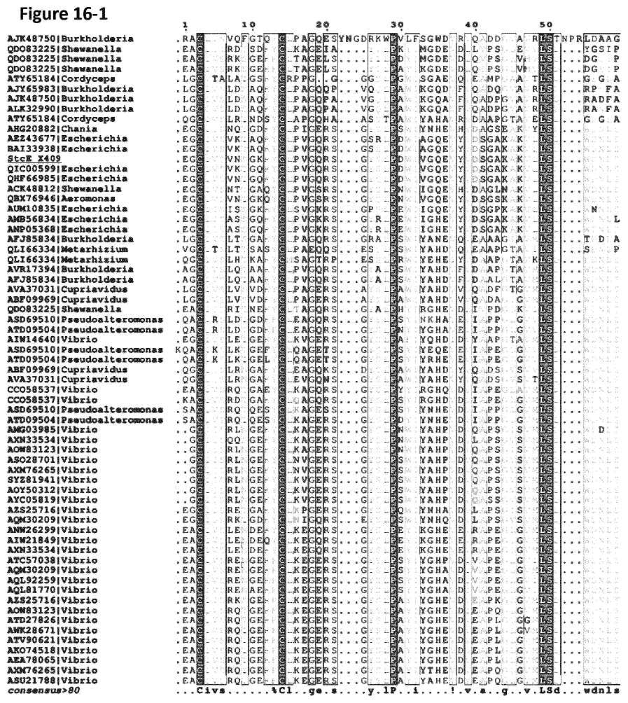

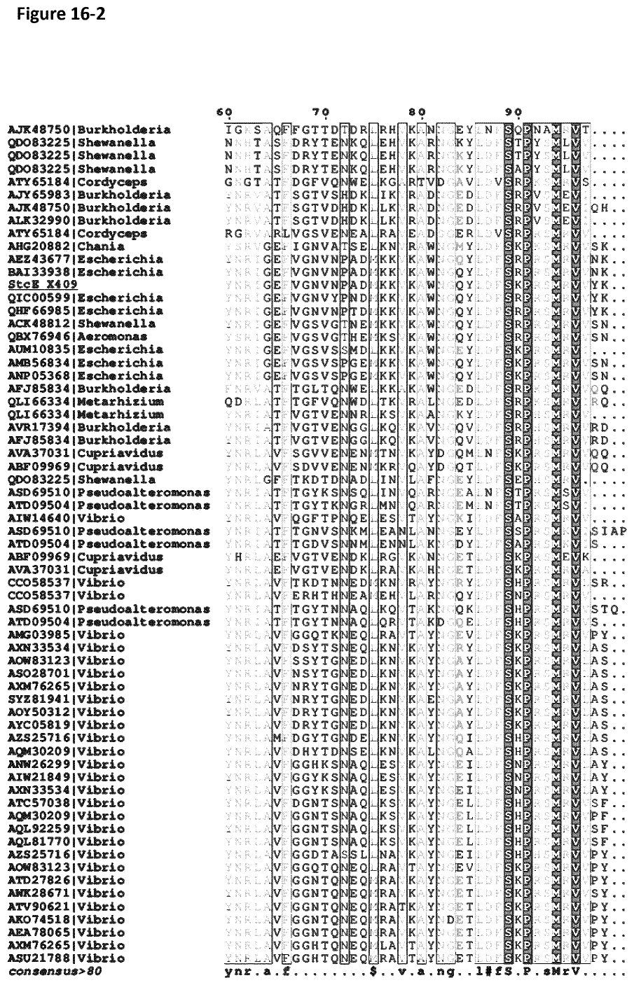

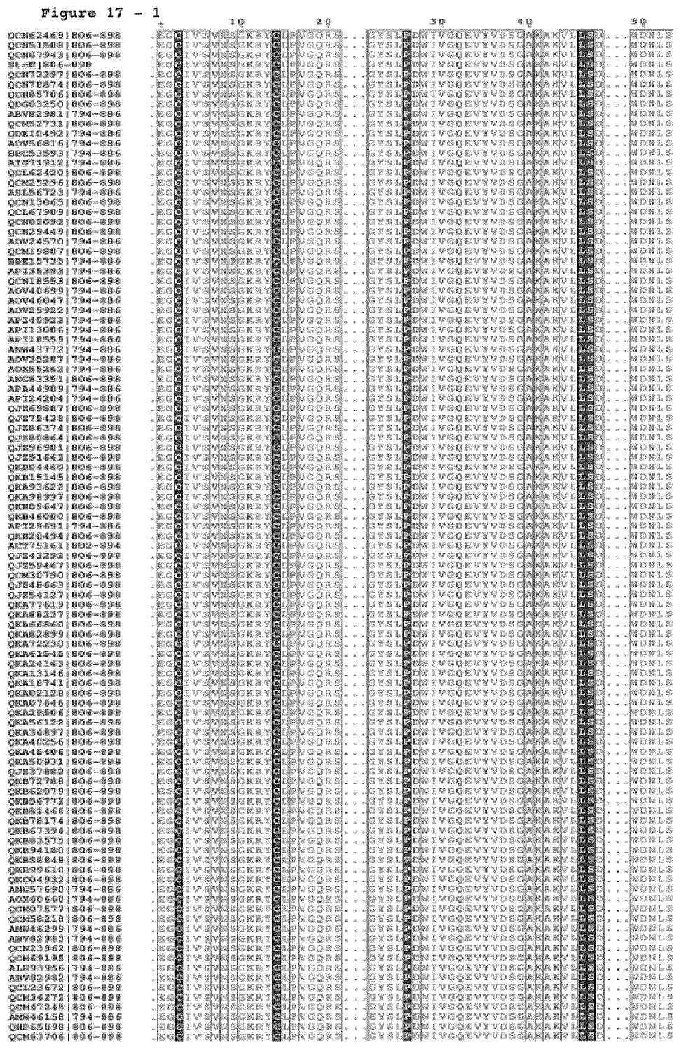

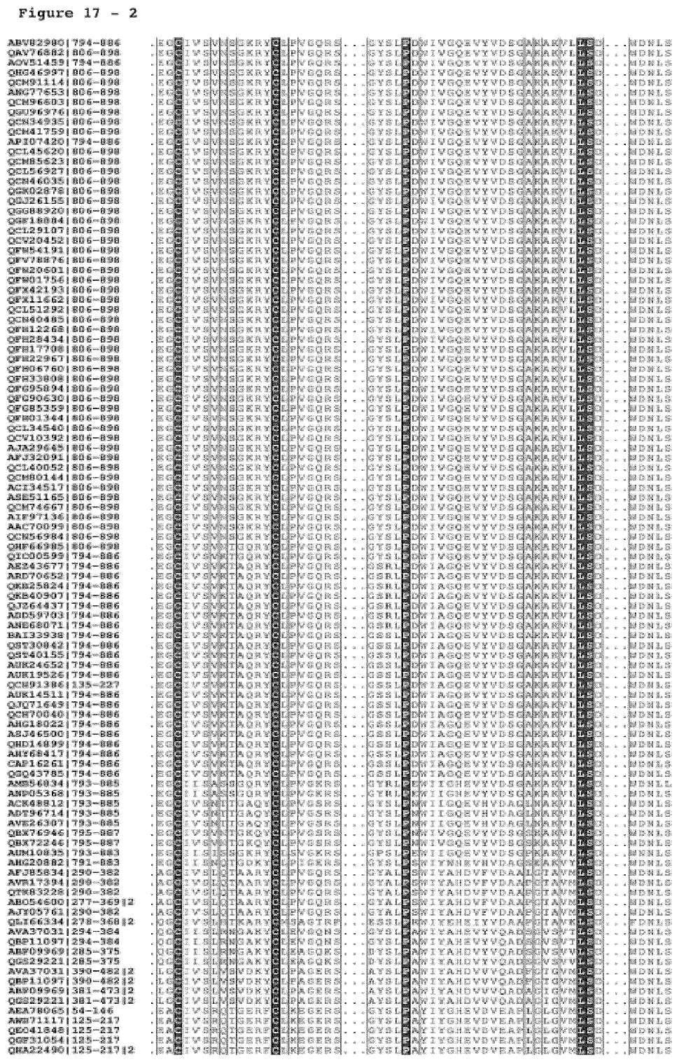

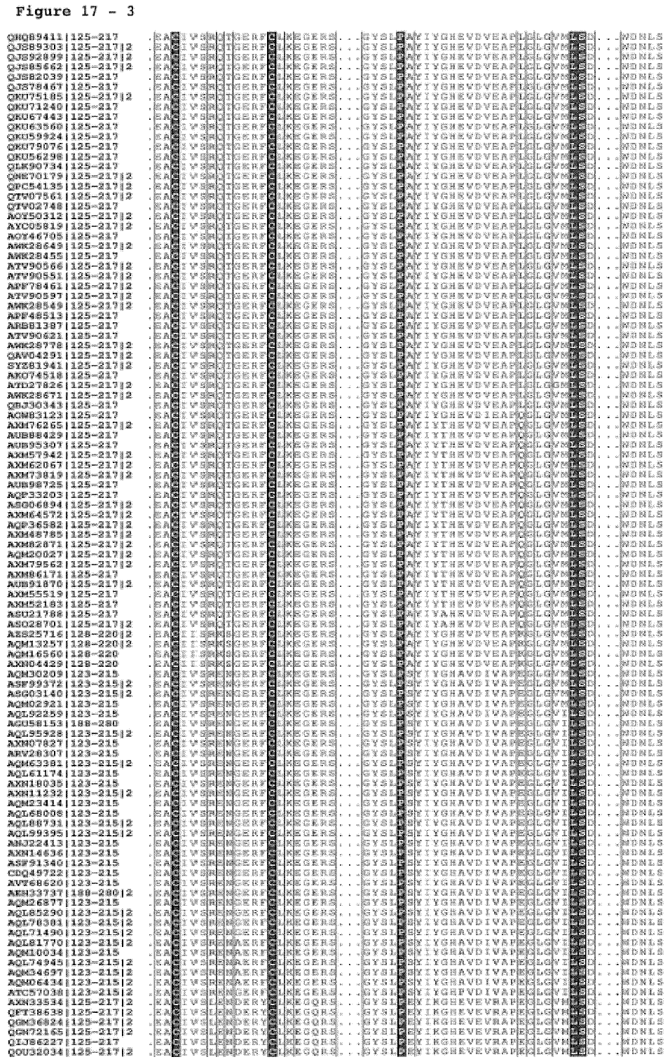

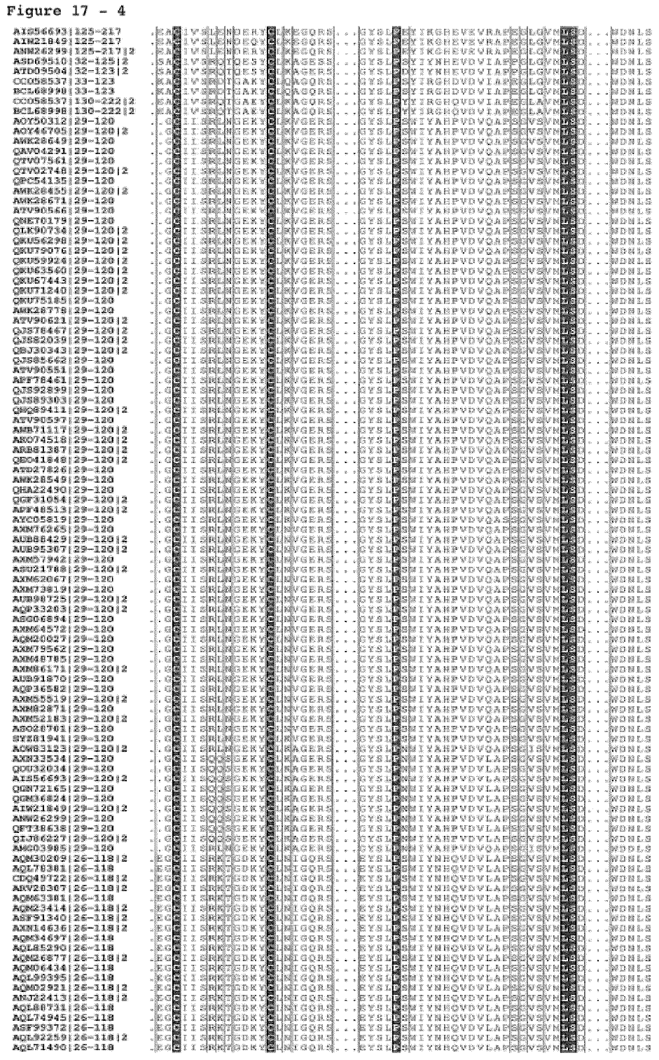

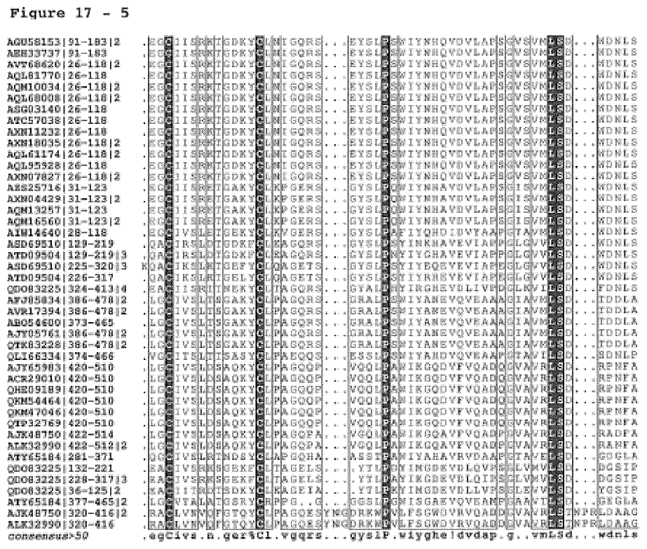

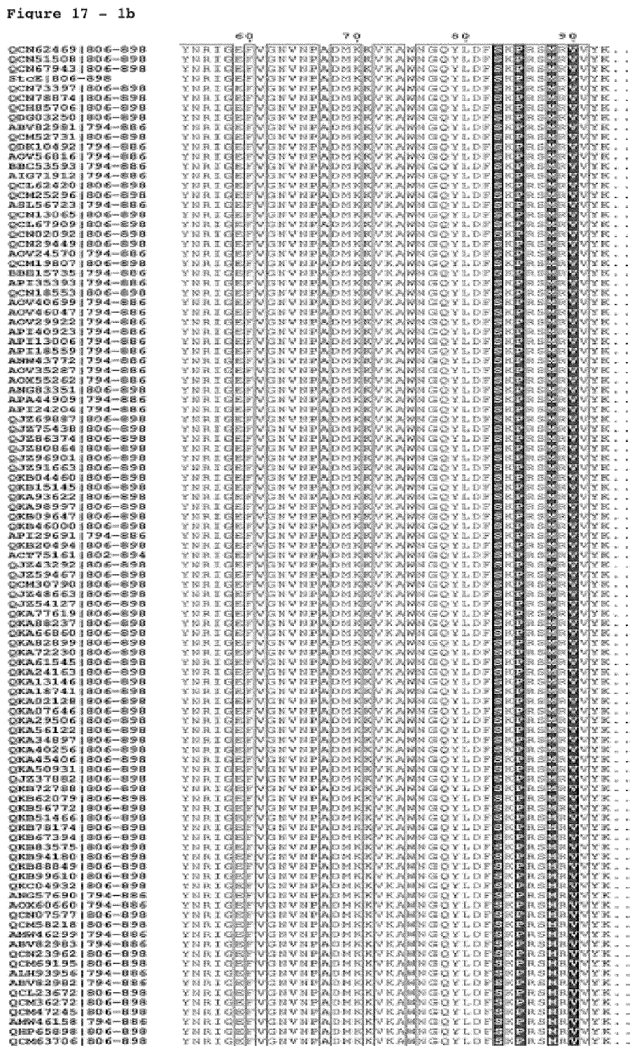

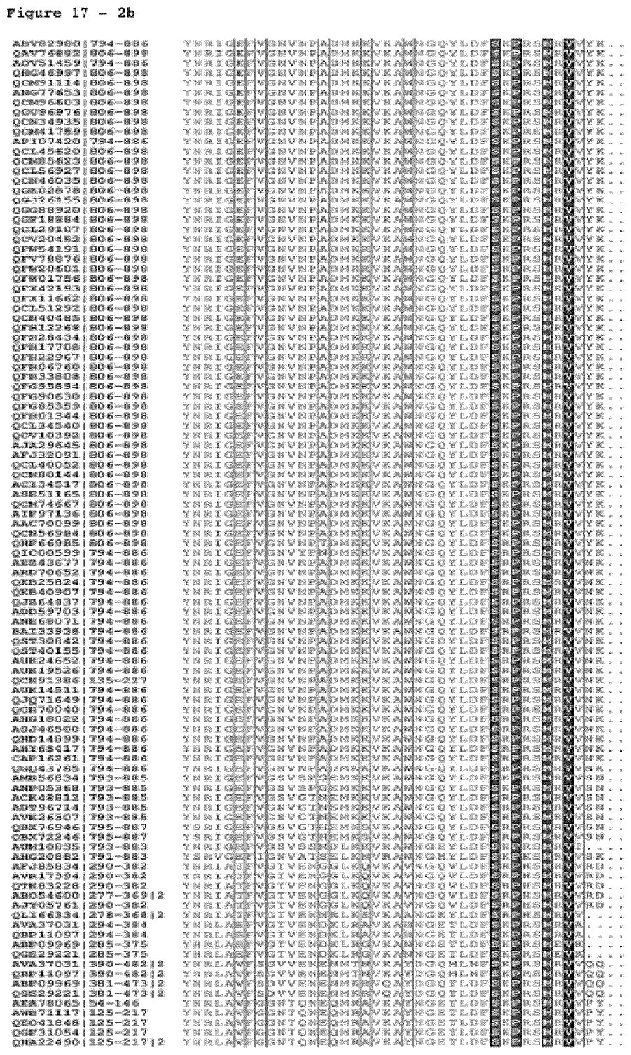

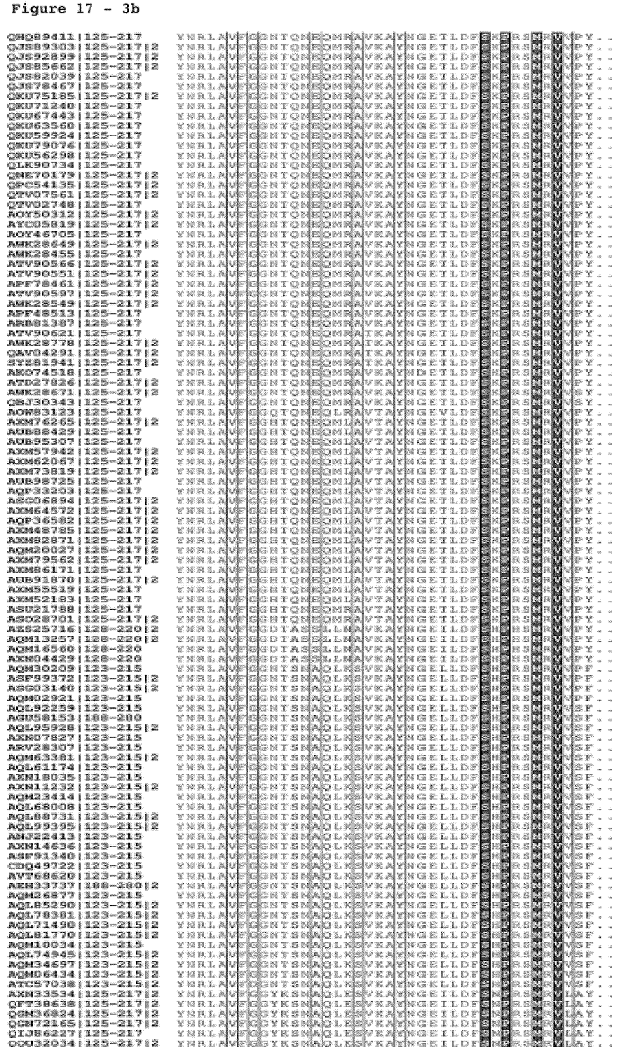

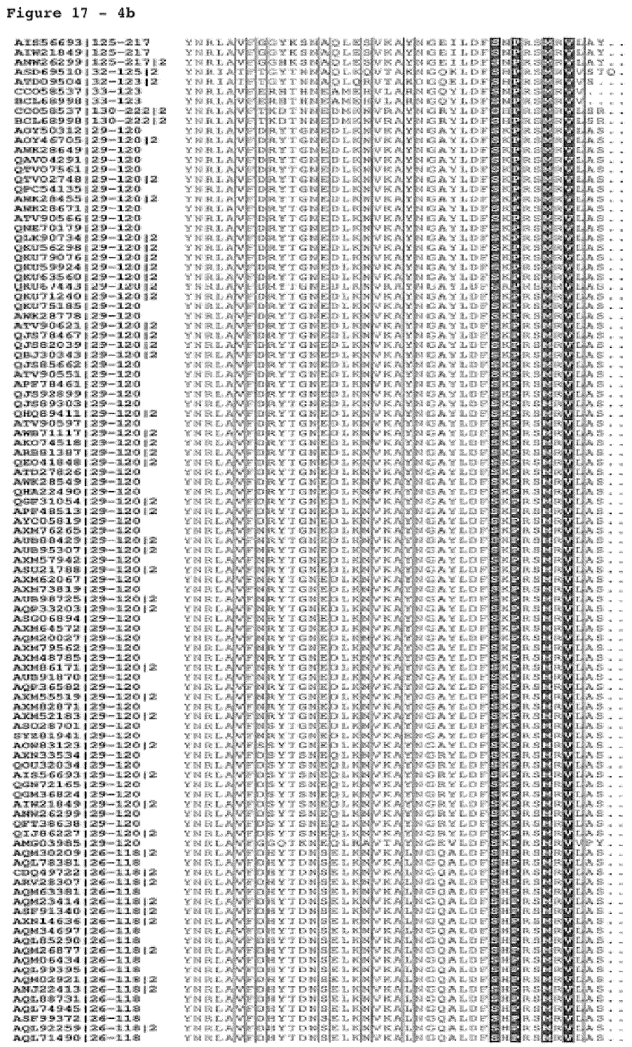

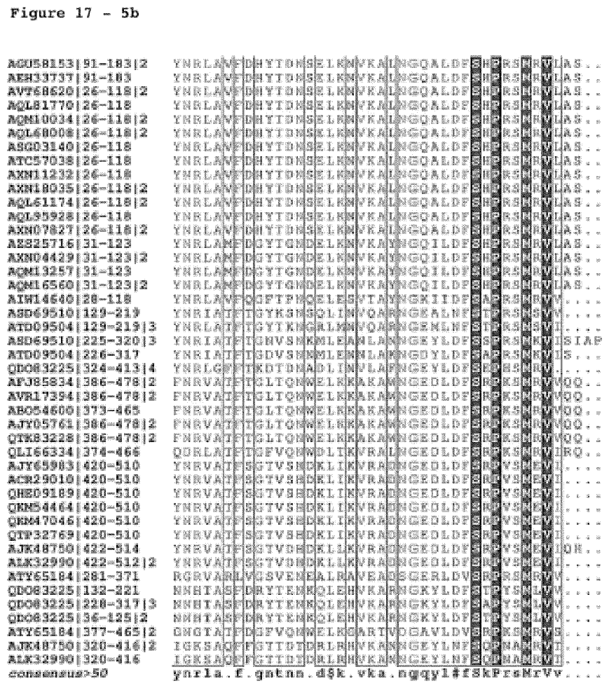

Example 4—The family of X409 mucin-binding modules. The 3-D structure of StcE24 was used to identify the boundaries of the X409 mucin-binding module from Escherichia coli O157:H7 (FIG. 14). The amino acid sequence of the identified X409 mucin-binding module was used to search related sequences Using BlastP⁷³ with default parameters against the non-redundant protein sequence database of the NCBI. Sequences similar to that of the X409 module were identified in a large variety of different types of proteins and strains of bacteria (FIG. 15). Analysis of 60 closely related X409 module sequences revealed highly conserved amino acids and domain features that indicates related functional properties (FIG. 16). Wider analysis of 400 X409-related modules further support the conserved sequence and domain features of the X409 mucin-binding modules (FIG. 17). The X409 module sequence of Escherichia coli O157:H7 StcE protease exhibits between 100% and 65% amino acid sequence identity to related modules found in Zn-metalloproteases with Pfam10462 domains. The X409 module sequence of Escherichia coli O157:H7 StcE exhibits 35-100% amino acid sequence identity with X409 modules found in other bacteria.

To further validate that the identified X409 related sequences represents genuine functional mucin-binding modules, four predicted X409 modules were selected from the wide range of identified X409 related sequences (FIG. 15-17). The four related X409 modules were derived from:
Vibrio anaquillarum (Gene Bank accession number AZS25716) with 56% sequence identity to the Escherichia coli O157 StcE X409,
Aeromonas hydrophilia (accession number QBX76946) with 84% sequence identity to the E. coli O157 StcE X409,
Shewanella baltica OS223 (accession number ACK48812) with 79% sequence identity to the E. coli StcE X409,
E. coli (accession number AUM10835) with 80% sequence identity to the E. coli StcE X409.

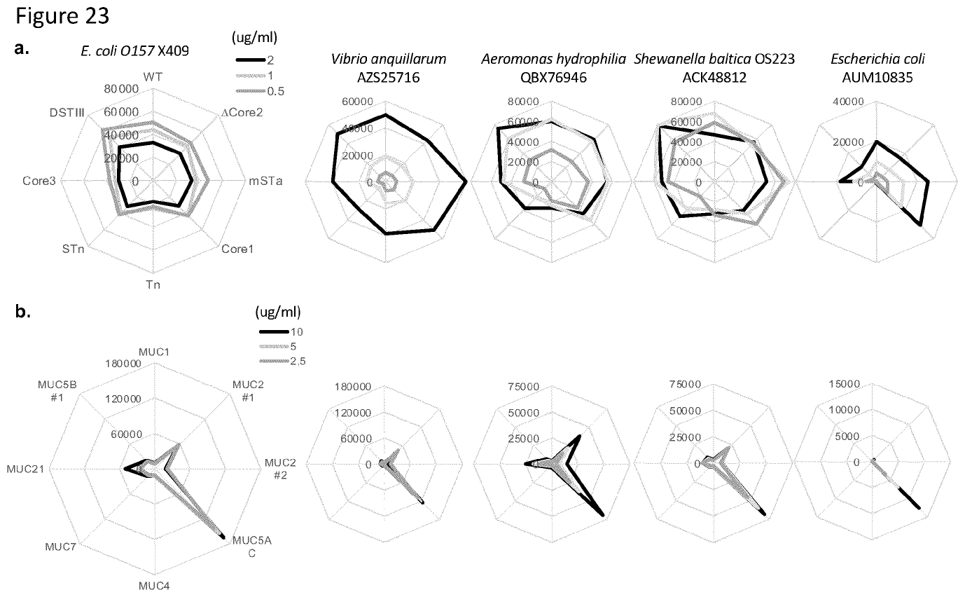

Shown in FIG. 23 is flow cytometry analysis of the mucin-binding properties of these four X409 variant sequence modules in comparison to StcE X409. All four modules exhibit strong binding to HEK293 cells displaying the MUC5Ac reporter with different O-glycans similarly to StcE X409 (FIG. 23a). Notable differences is that the variant E. coli (accession number AUM10835) module did not bind to the simplest Tn and STn O-glycoforms, which illustrates that this X409 module has improved selectivity for the mature elaborated O-glycoforms of MUC5Ac compared to other X409 modules.

Notably, binding to the most immature glycoforms is preferable for example when using the mucin-binding properties of X409 to target or deliver substances to the most nascent mucin layers in mucosal linings. Binding to the immature glycoforms of mucins is for example not preferable when using the mucin-binding properties of X409 to target or deliver substances to mucosal linings such as the gut where bacteria continuously degrade the glycans resulting in appearance of for example Tn glycoforms at the most superficial mucus layers and in shed mucins. Thus, the ability of X409 variants to selectively (StcE X409 and other variants) or exclusive (E. coli X409 accession number AUM10835) is preferable to target deeper mucus layers in the gut mucosa and prevent adherence to the superficial and shed mucin layers. All four modules exhibit selective mucin-binding preference for the mucin MUC5Ac (HEK293 cells displaying the MUC5Ac reporter with core1 T O-glycans) similarly to StcE X409 (FIG. 23b).

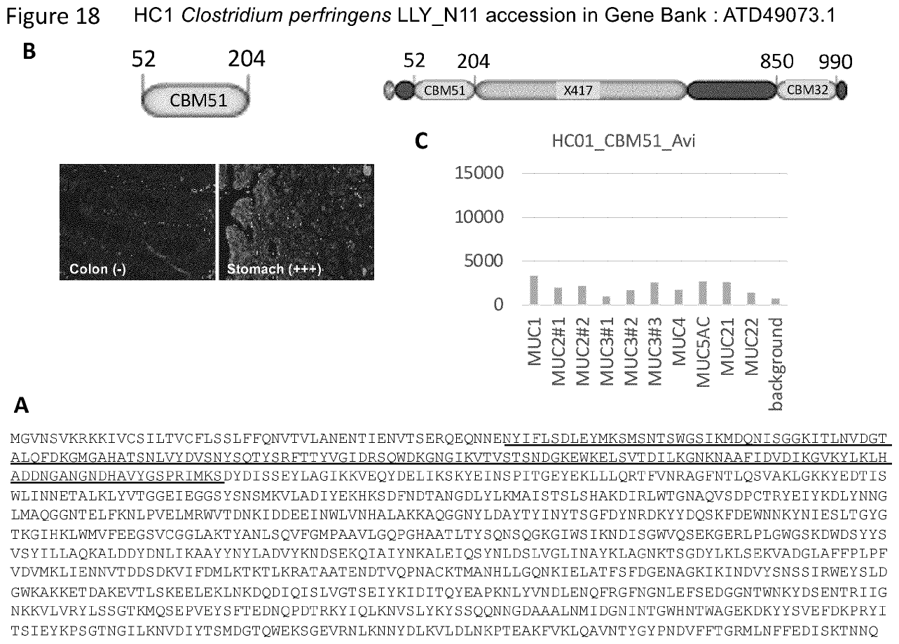

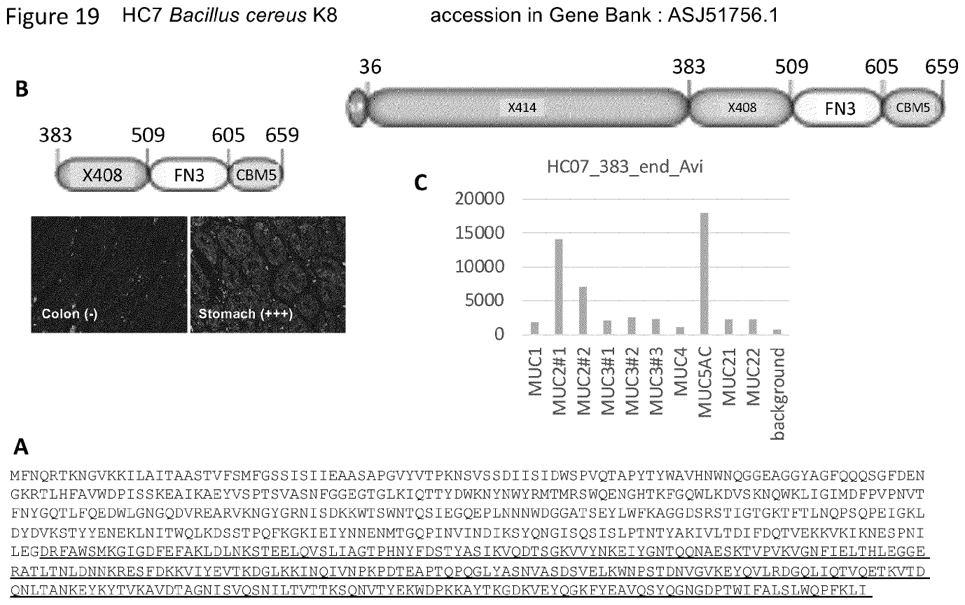

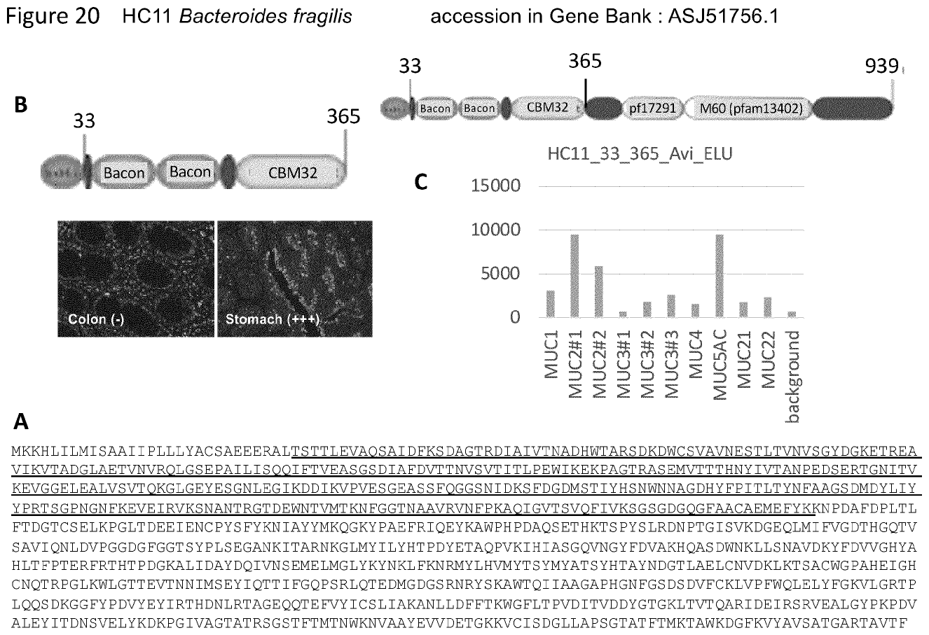

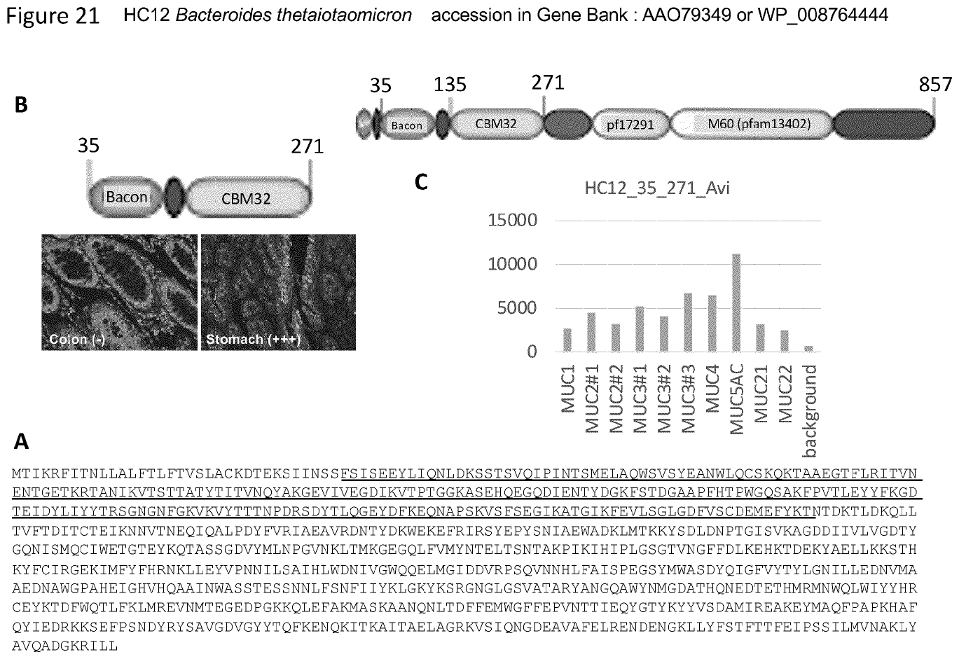

Example 5—Novel families of mucin-binding modules unrelated to the X409 sequence. We next searched for CBM-related modules associated with peptidases. The CBM families that were searched were those listed in the Carbohydrate-Active Enzymes database www.cazy.org;⁷⁵ BlastP⁷³ was used to identify proteins with similarity to representative CBM sequences in the non-redundant protein sequence database of the NCBI using default parameters, followed by the identification of domains related to peptidases using Pfam⁷⁴ using default parameters. This resulted in identification of diverse sequence modules with sequences unrelated to X409, but with similar characteristics of distinct modules positioned N- or C-terminal to predicted peptidase domains (FIGS. 18-21). Testing these modules by immunohistology on human stomach and colon tissue sections revealed different binding patterns than X409, and analysis of the mucin-binding properties by the mucin TR display revealed distinct binding profiles to select mucin TRs different from X409 (FIGS. 18-21).

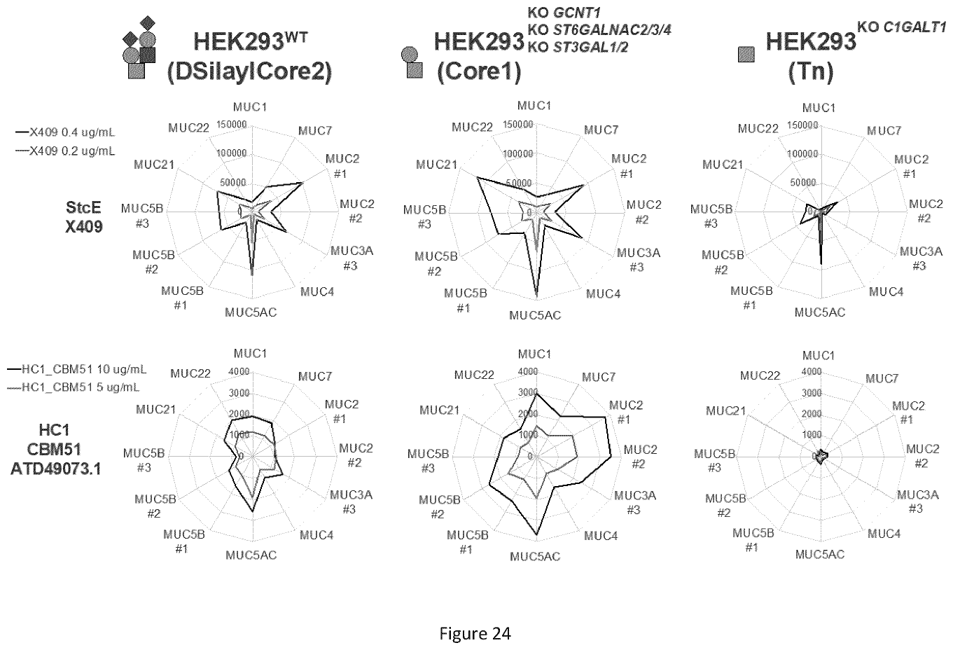

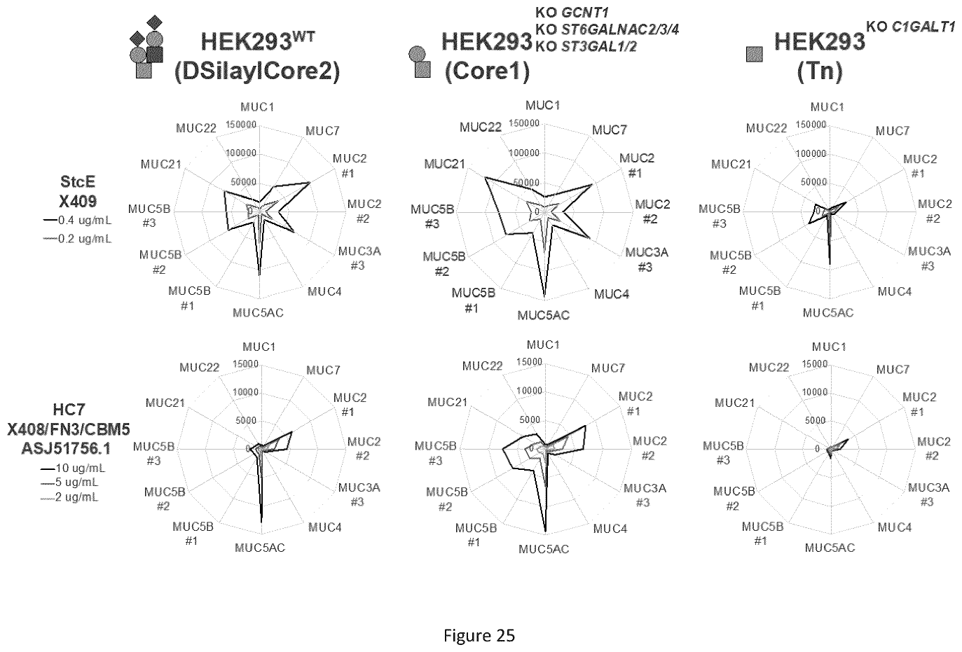

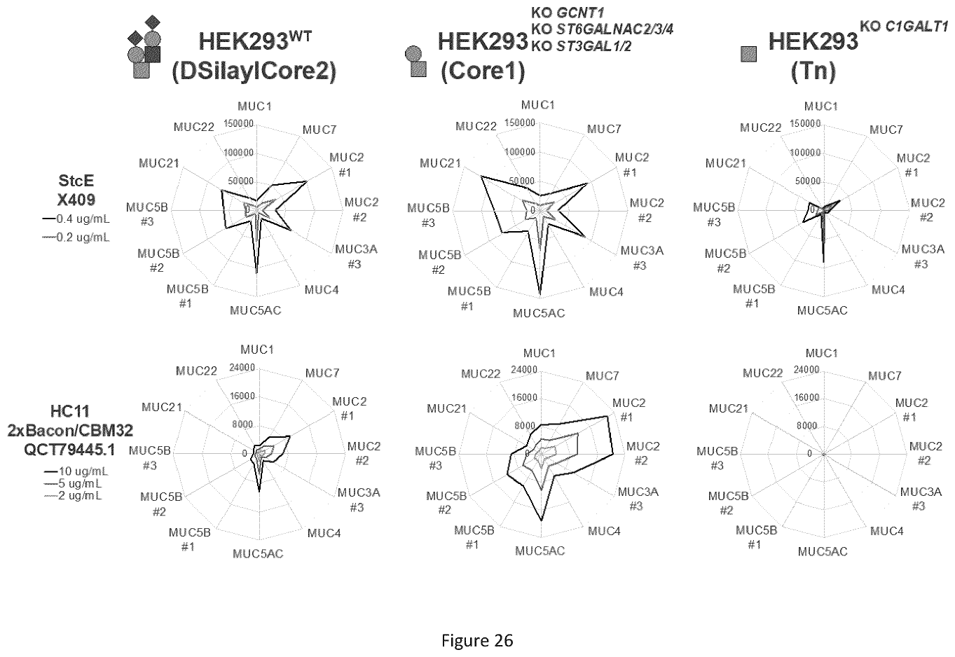

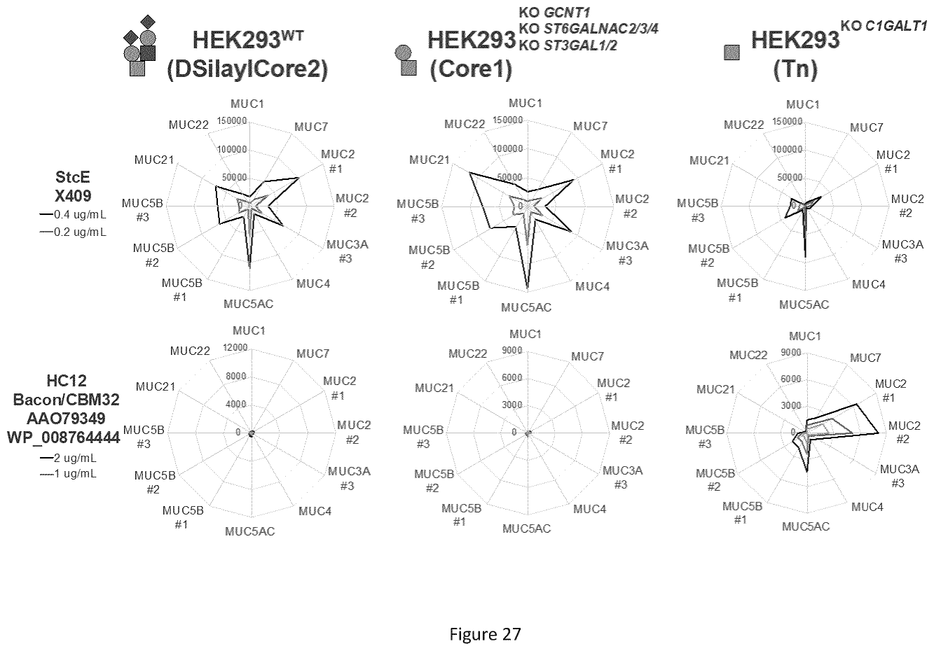

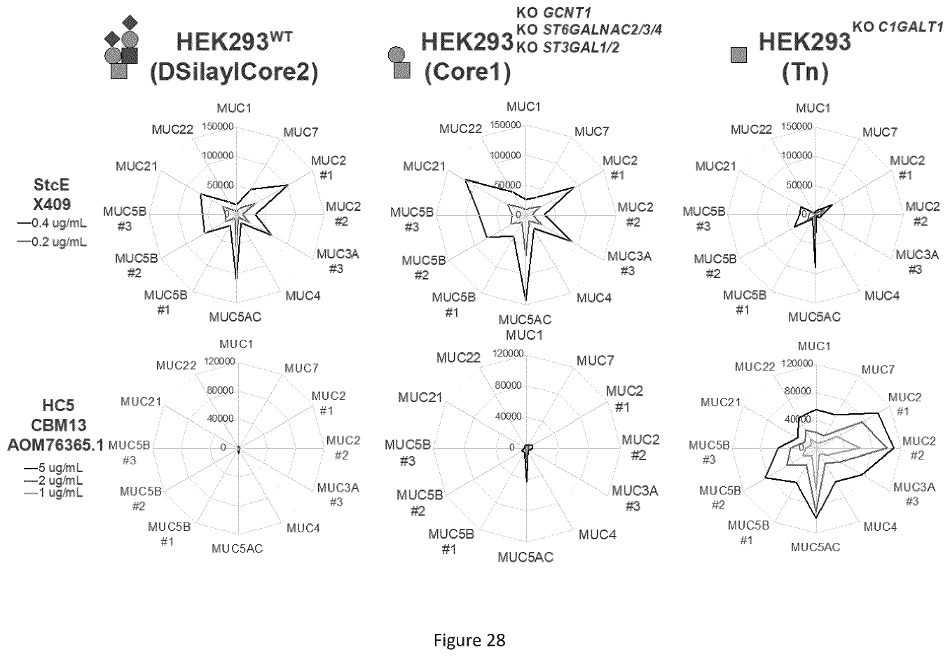

A detailed analysis of the mucin-binding properties of the novel modules were performed by flow cytometry with HEK293 cells displaying mucin reporters with three different O-glycan structures (Disialyl-core2, core1 T, and Tn) (FIG. 24-28). StcE X409 exhibits preference for elaborated O-glycans and select mucins, while HC1 (CBM51) shows broader mucin reactivity (including e.g. MUC1) but without reactivity to the Tn O-glycoforms (FIG. 24). HC7 (X408/FN3/CBM5) in contrast shows highly selective binding to MUC5Ac and MUC2 with only limited reactivity with these carrying Tn O-glycans (FIG. 25). HC11 (2×Bacon/CBM32) shows similar mucin preference as HC7 but preference for the core1 T O-glycans structure. HC12 (Bacon/CBM32) shows exclusive binding to the Tn glycoforms of MUC5Ac and MUC2. HC5 (CBM13) shows exclusive binding to the Tn glycoforms as HC12, but with wider binding to different mucins.

These results clearly demonstrate that unique mucin-binding modules can be identified among X409 unrelated sequences, and that these have different mucin-binding properties with selectivity for different mucins and O-glycan structures. Thus, the X409-related and unrelated mucin-binding modules presented here offers a variety of targeting modules characterized by their unique binding properties for select mucins and O-glycans that are useful for targeting and delivery.

Example 6—Use of Mucin-binding Modules to Target/Deliver to Mucosal Surfaces

The mucin-binding modules are valuable for targeting and delivery of substances to mucosal surfaces and the distinct mucin and glycan binding properties of the X409-related and unrelated mucin-binding modules can be used to custom-design and tune targeting to different mucosal surfaces expressing different mucins and O-glycans. The mucin-binding modules can for example be used to target orally delivered substances to the gut mucosa, inhaled substances to the respiratory mucosa, and topically administered substances to for example the vaginal and nasal lining mucosa. Preferable substances for delivery to mucosal surfaces include bioactive peptides including peptide hormones, small molecule drugs, enzymes, vaccine compositions, RNA and DNA. Delivery of enzymes may include therapeutic enzymes, for example digestive enzymes needed following surgical removal of pancreas, and enzymes used for improving feed digestion and uptake of nutrients.

The mucin-binding modules are used in construction of conjugates, complexes and lipid particles where the mucin-binding module is chemically linked, complexed with, adsorbed to, or incorporated into for example lipid particles. For example by chemical conjugation of a lipid moiety to the mucin-binding module this lipid modified module is inserted into lipid particles that may contain substances, and such coated particles adhere to and are retained at the mucosal surface.

The mucin-binding modules are used in chimeric fusion protein designs where the mucin-binding module is added to a bioactive protein, such as therapeutic protein, bioactive peptide, or enzyme, by recombinant gene technologies to for example mediate binding to the mucosa and enhance residence time, biodistribution, and bioactive effects. The mucin-binding modules are for example used to enhance efficiency of enzymes like proteases, lipases, phytases, amylase, xylanases, β-Glucanases, α-Galactosidases, mannanases, cellulases, hemicellulases, and pectinases.

One example of delivery of fusion protein drug is protein sequences built on a rigid alpha-helicoidal HEAT-like protein sequences (αReps) that recognize the SARS-COV-2 spike receptor ACE2 binding domain and neutralize virus infection. Installation of such αReps in the nasal cavity before or during infections effectively reduce the replication of a SARS-COV-2 strain in the nasal epithelium in hamsters. The αRep protein localize to the surface mucosa and is detectable for 0-30 min by immunohistological analysis using antibodies to tags, but at 60 min the protein is barely detectable. By introducing X409 to the αRep protein the chimeric fusion protein will remain detectable at the surface considerably longer (for example up to 6 hours) after nasal instillation and thereby provide longer bioactivity and inhibition of viral infection. For example the αRep F9-C2 protein or X409 fusion protein hereof may be given as a prophylactic to limit SARS-COV-2 infection in vivo. Syrian golden hamsters that reflect the infection in human may be pretreated with for example 0.6 mg of the proteins distributed between the two nostrils 1h prior to infection with SARS-COV-2, and the presence of infiltrated αReps on the surface epithelium layer will be observed indicating an efficient absorption of the molecule.

The X409 mucin-binding module is further useful for improving mucosal vaccine delivery and effectiveness. Mucosal vaccine formulations are dependent on uptake and presentation by resident mucosal innate immune cells and antigen presenting cells, and topical or inhaled (for example by spray) formulations of vaccines are improved by extending their residence time at the mucosal surface. The X409 mucin-binding module is useful to attach by covalent or non-covalent methods to a vaccine formulation or in chimeric fusion protein designs of vaccines comprising for example recombinant proteins or included in the coding region of RNA and DNA vaccine designs to improve adhesion to oral, nasal and other respiratory mucosal surfaces and significantly enhance residence time and effectiveness. The X409 module may be incorporated in RNA and DNA vaccine designs by introducing the coding region for X409 as outlined in this invention in the design (for example before, after, or separate) to the coding region for the protein immunogen of interest. The X409 module may also be conjugated and/or incorporated in the delivery vehicle for mucosal RNA and DNA vaccine formulations to allow the formulation to adhere and reside for extended periods at the mucosal surface for effective delivery of vaccines. The X409 module may also be used for protein, glycoprotein and polysaccharide vaccines by conjugating, incorporating and/or fusing the X409 module to recombinant vaccines in order to enhance mucosal adhesions and effectiveness.

### SEQUENCE HEADERS Included as Clear Text

<110> University of Copenhagen

<120> PEPTIDES WITH MUCIN-BINDING PROPERTIES

<130> P77084EP

<160> 5

<170> BISSAP 1.3.6

<210> 1

<211> 93

<212> PRT

<213>Escherichia coli

<223> X409 module of StcE

<210> 2

<211> 153

<212> PRT

<213> Clostridium perfringens

<223> HC1 CBM51

<210> 3

<211> 276

<212> PRT

<213> Bacillus cereus

<223> HC7 X408-FN3-CBM5 of K8

<210> 4

<211> 336

<212> PRT

<213> Bacteroides fragilis

<223> HC11 Bacon-Bacon-CBM32

<210> 5

<211> 237

<212> PRT

<213> Bacteroides fragilis

<223> HC12 Bacteroides thetaiotaomicron

### REFERENCES CITED

1. Hansson, G. C. Mucus and mucins in diseases of the intestinal and respiratory tracts. J. Intern. Med. 285, 479-490 (2019).

2. Werlang, C., Cárcarmo-Oyarce, G. & Ribbeck, K. Engineering mucus to study and influence the microbiome. Nat Rev Mater 4, 134-145 (2019).

3. Sonnenburg, J. L., Angenent, L. T. & Gordon, J. I. Getting a grip on things: how do communities of bacterial symbionts become established in our intestine? Nat. Immunol. 5, 569-573 (2004).

4. McLoughlin, K., Schluter, J., Rakoff-Nahoum, S., Smith, A. L. & Foster, K. R. Host Selection of Microbiota via Differential Adhesion. Cell Host Microbe 19, 550-559 (2016).

5. Johansson, M. E. V., Sjövall, H. & Hansson, G. C. The gastrointestinal mucus system in health and disease. Nat Rev Gastroenterol Hepatol 10, 352-361 (2013).

6. Link, T. et al. Bioprocess development for the production of a recombinant MUC1 fusion protein expressed by CHO-K1 cells in protein-free medium. Journal of Biotechnology 110, 51-62 (2004).

7. Corfield, A. P. Mucins: a biologically relevant glycan barrier in mucosal protection. Biochim. Biophys. Acta 1850, 236-252 (2015).

8. Marcos-Silva, L. et al. Characterization of binding epitopes of CA125 monoclonal antibodies. Journal of Proteome Research 13, 3349-3359 (2014).

9. Steentoft, C. et al. Precision mapping of the human O-GalNAc glycoproteome through SimpleCell technology. EMBO J. 32, 1478-1488 (2013).

10. Lang, T., Hansson, G. C. & Samuelsson, T. Gel-forming mucins appeared early in metazoan evolution. Proceedings of the National Academy of Sciences 104, 16209-16214 (2007).

11. Thornton, D. J., Rousseau, K. & McGuckin, M. A. Structure and function of the polymeric mucins in airways mucus. Annu Rev Physiol 70, 459-486 (2008).

12. Hansson, G. C. Mucins and the Microbiome. Annu. Rev. Biochem. 89, 769-793 (2020).

13. Liu, J. et al. SEDDS for intestinal absorption of insulin: Application of Caco-2 and Caco-2/HT29 co-culture monolayers and intra-jejunal instillation in rats. Int J Pharm 560, 377-384 (2019).

14. Levery, S. B. et al. Advances in mass spectrometry driven O-glycoproteomics. Biochim. Biophys. Acta 1850, 33-42 (2015).

15. Ali, L. et al. The O-glycomap of lubricin, a novel mucin responsible for joint lubrication, identified by site-specific glycopeptide analysis. Mol. Cell Proteomics 13, 3396-3409 (2014).

16. Khoo, K. H. Advances toward mapping the full extent of protein site-specific O-GalNAc glycosylation that better reflects underlying glycomic complexity. Curr. Opin. Struct. Biol. 56, 146-154 (2019).

17. Goletz, S. et al. A sequencing strategy for the localization of O-glycosylation sites of MUC1 tandem repeats by PSD-MALDI mass spectrometry. Glycobiology 7, 881-896 (1997).

18. Hanisch, F. G., Green, B. N., Bateman, R. & Peter-Katalinić, J. Localization of O-glycosylation sites of MUC1 tandem repeats by QTOF ESI mass spectrometry. J Mass Spectrom 33, 358-362 (1998).

19. Kinarsky, L. et al. Conformational studies on the MUC1 tandem repeat glycopeptides: implication for the enzymatic O-glycosylation of the mucin protein core. Glycobiology 13, 929-939 (2003).

20. Ali, L. et al. The O-glycomap of lubricin, a novel mucin responsible for joint lubrication, identified by site-specific glycopeptide analysis. Mol. Cell Proteomics 13, 3396-3409 (2014).

21. Lathem, W. W. et al. StcE, a metalloprotease secreted by Escherichia coli O157:H7, specifically cleaves C1 esterase inhibitor. Mol. Microbiol. 45, 277-288 (2002).

22. Grys, T. E., Siegel, M. B., Lathem, W. W. & Welch, R. A. The StcE protease contributes to intimate adherence of enterohemorrhagic Escherichia coli O157:H7 to host cells. Infect. Immun. 73, 1295-1303 (2005).

23. Malaker, S. A. et al. The mucin-selective protease StcE enables molecular and functional analysis of human cancer-associated mucins. Proc. Natl. Acad. Sci. U.S.A. 116, 7278-7287 (2019).

24. Yu, A. C. Y., Worrall, L. J. & Strynadka, N. C. J. Structural insight into the bacterial mucinase StcE essential to adhesion and immune evasion during enterohemorrhagic E. coli infection. Structure 20, 707-717 (2012).

25. Shon, D. J. et al. An enzymatic toolkit for selective proteolysis, detection, and visualization of mucin-domain glycoproteins. Proc. Natl. Acad. Sci. U.S.A. 117, 21299-21307 (2020).

26. Grys, T. E., Walters, L. L. & Welch, R. A. Characterization of the StcE protease activity of Escherichia coli O157:H7. J. Bacteriol. 188, 4646-4653 (2006).

27. Walsham, A. D. S. et al. Lactobacillus reuteri Inhibition of Enteropathogenic Escherichia coli Adherence to Human Intestinal Epithelium. Front Microbiol 7, 244 (2016).

28. Malaker, S. A. et al. Revealing the human mucinome. bioRxiv 2021.01.27.428510 (2021). doi:10.1101/2021.01.27.428510

29. Kudelka, M. R. et al. Cellular O-Glycome Reporter/Amplification to explore O-glycans of living cells. Nature Methods 7, 618-86 (2015).

30. Blixt, O. et al. A high-throughput O-glycopeptide discovery platform for seromic profiling. Journal of Proteome Research 9, 5250-5261 (2010).

31. Kramer, J. R., Onoa, B., Bustamante, C. & Bertozzi, C. R. Chemically tunable mucin chimeras assembled on living cells. Proc. Natl. Acad. Sci. U.S.A. 112, 12574-12579 (2015).

32. Petrou, G. & Crouzier, T. Mucins as multifunctional building blocks of biomaterials. Biomater Sci 6, 2282-2297 (2018).

33. Chen, Y. H. et al. The GAGOme: a cell-based library of displayed glycosaminoglycans. Nature Methods 15, 881-888 (2018).

34. Narimatsu, Y. et al. An Atlas of Human Glycosylation Pathways Enables Display of the Human Glycome by Gene Engineered Cells. Mol. Cell 75, 394-407.e5 (2019).

35. Genetic glycoengineering in mammalian cells. J. Biol. Chem. 100448 (2021). doi:10.1016/j.jbc.2021.100448

36. Büll, C., Joshi, H. J., Clausen, H. & Narimatsu, Y. Cell-Based Glycan Arrays—A Practical Guide to Dissect the Human Glycome. STAR Protocols 1, 100017 (2020).

37. Cohen, M. & Varki, A. Modulation of glycan recognition by clustered saccharide patches. Int Rev Cell Mol Biol 308, 75-125 (2014).

38. Varki, A. Selectin ligands. Proceedings of the National Academy of Sciences 91, 7390-7397 (1994).

39. Rillahan, C. D. & Paulson, J. C. Glycan microarrays for decoding the glycome. Annu. Rev. Biochem. 80, 797-823 (2011).

40. Cummings, R. D. The repertoire of glycan determinants in the human glycome. Molecular BioSystems 5, 1087-1104 (2009).

41. Blixt, O. et al. Printed covalent glycan array for ligand profiling of diverse glycan binding proteins. Proceedings of the National Academy of Sciences 101, 17033-17038 (2004).

42. Cummings, R. D. et al. Sialic Acids and Other Nonulosonic Acids. (2015). doi:10.1101/glycobiology.3e.015

43. Schjoldager, K. T., Narimatsu, Y., Joshi, H. J. & Clausen, H. Global view of human protein glycosylation pathways and functions. Nat Rev Mol Cell Biol 21, 729-749 (2020).

44. Varki, A. et al. Symbol Nomenclature for Graphical Representations of Glycans. Glycobiology 25, 1323-1324 (2015).

45. Huson, D. H. & Scornavacca, C. Dendroscope 3: an interactive tool for rooted phylogenetic trees and networks. Syst Biol 61, 1061-1067 (2012).

46. Edgar, R. C. MUSCLE: multiple sequence alignment with high accuracy and high throughput. Nucleic Acids Res. 32, 1792-1797 (2004).

47. Gouet, P., Courcelle, E., Stuart, D. I. & Métoz, F. ESPript: analysis of multiple sequence alignments in PostScript. Bioinformatics 15, 305-308 (1999).

48. Narimatsu, Y. et al. A validated gRNA library for CRISPR/Cas9 targeting of the human glycosyltransferase genome. Glycobiology 28, 295-305 (2018).

49. Lonowski, L. A. et al. Genome editing using FACS enrichment of nuclease-expressing cells and indel detection by amplicon analysis. Nature Protocols 12, 581-603 (2017).

50. Pinto, R. et al. Precise integration of inducible transcriptional elements (PrlITE) enables absolute control of gene expression. Nucleic Acids Res. 45, e123-e123 (2017).

51. Yang, Z. et al. Fast and sensitive detection of indels induced by precise gene targeting. Nucleic Acids Res. 43, e59-e59 (2015).

52. Vakhrushev, S. Y., Dadimov, D. & Peter-Katalinić, J. Software platform for high-throughput glycomics. Anal. Chem. 81, 3252-3260 (2009).

53. Ricardo, S. et al. Detection of glyco-mucin profiles improves specificity of MUC16 and MUC1 biomarkers in ovarian serous tumours. Mol Oncol 9, 503-512 (2015).

54. Varki, A. Biological roles of glycans. Glycobiology 27, 3-49 (2017).

55. Palma, A. S., Feizi, T., Childs, R. A., Chai, W. & Liu, Y. The neoglycolipid (NGL)-based oligosaccharide microarray system poised to decipher the meta-glycome. Curr Opin Chem Biol 18, 87-94 (2014).

56. Hollingsworth, M. A. & Swanson, B. J. Mucins in cancer: protection and control of the cell surface. Nat. Rev. Cancer 4, 45-60 (2004).

57. Narimatsu, Y. et al. Exploring Regulation of Protein O-Glycosylation in Isogenic Human HEK293 Cells by Differential O-Glycoproteomics. Mol. Cell Proteomics 18, 1396-1409 (2019).

58. Steentoft, C. et al. Mining the O-glycoproteome using zinc-finger nuclease-glycoengineered SimpleCell lines. Nature Methods 8, 977-982 (2011).

59. Burchell, J., Taylor-Papadimitriou, J., Boshell, M., Gendler, S. & Duhig, T. A short sequence, within the amino acid tandem repeat of a cancer-associated mucin, contains immunodominant epitopes. Int. J. Cancer 44, 691-696 (1989).

60. Tarp, M. A. et al. Identification of a novel cancer-specific immunodominant glycopeptide epitope in the MUC1 tandem repeat. Glycobiology 17, 197-209 (2007).

61. Reis, C. A. et al. Development and characterization of an antibody directed to an alpha-N-acetyl-D-galactosamine glycosylated MUC2 peptide. Glycoconj. J. 15, 51-62 (1998).

62. Remmers, N. et al. Aberrant expression of mucin core proteins and o-linked glycans associated with progression of pancreatic cancer. Clin. Cancer Res. 19, 1981-1993 (2013).

63. Marcos, N. T. et al. Role of the Human ST6GalNAc-I and ST6GaINAc-II in the Synthesis of the Cancer-Associated Sialyl-Tn Antigen. Cancer Res. 64, 7050-7057 (2004).

64. Sewell, R. et al. The ST6GaINAc-I sialyltransferase localizes throughout the Golgi and is responsible for the synthesis of the tumor-associated sialyl-Tn O-glycan in human breast cancer. J. Biol. Chem. 281, 3586-3594 (2006).

65. Lathem, W. W., Bergsbaken, T., Witowski, S. E., Perna, N. T. & Welch, R. A. Acquisition of stcE, a C1 esterase inhibitor-specific metalloprotease, during the evolution of Escherichia coliO157:H7. J. Infect. Dis. 187, 1907-1914 (2003).

66. Szabady, R. L., Welch, R. A. Handbook of Proteolytic Enzymes Vol.3 Ch 286 (Academic Press, 30 Oct. 2012).

67. Szabady, R. L., Lokuta, M. A., Walters, K. B., Huttenlocher, A. & Welch, R. A. Modulation of neutrophil function by a secreted mucinase of Escherichia coli O157:H7. PLOS Pathog. 5, e1000320 (2009).

68. Johansson, M. E. V. et al. The inner of the two Muc2 mucin-dependent mucus layers in colon is devoid of bacteria. Proc. Natl. Acad. Sci. U.S.A. 105, 15064-15069 (2008).

69. Johansson, M. E. V., Larsson, J. M. H. & Hansson, G. C. The two mucus layers of colon are organized by the MUC2 mucin, whereas the outer layer is a legislator of host-microbial interactions. Proc. Natl. Acad. Sci. U.S.A. 108 Suppl 1, 4659-4665 (2011).

70. Holmén Larsson, J. M., Thomsson, K. A., Rodriguez-Piñeiro, A. M., Karlsson, H. & Hansson, G. C. Studies of mucus in mouse stomach, small intestine, and colon. III. Gastrointestinal Muc5ac and Muc2 mucin O-glycan patterns reveal a regiospecific distribution. Am. J. Physiol. Gastrointest. Liver Physiol. 305, G357-63 (2013).

71. Bergstrom, K. et al. Core 1- and 3-derived O-glycans collectively maintain the colonic mucus barrier and protect against spontaneous colitis in mice. Mucosal Immunol 10, 91-103 (2017).

72. Sharon, N. Lectins: carbohydrate-specific reagents and biological recognition molecules. J. Biol. Chem. 282, 2753-2764 (2007).

73. Altschul, S. F. et al. Gapped BLAST and PSI-BLAST: a new generation of protein database search programs. Nucleic Acids Res. 25, 3389-3402 (1997).

74. El-Gebali, S. et al. The Pfam protein families database in 2019. Nucleic Acids Res. 47, D427-D432 (2019).

75. Lombard, V., Golaconda Ramulu, H., Drula, E., Coutinho, P. M. & Henrissat, B. The carbohydrate-active enzymes database (CAZy) in 2013. Nucleic Acids Res. 42, D490-5 (2014).

### Items X

X1. A mucin-binding targeting agent comprising an isolated peptide selected from the group comprising

X409 peptide according to SEQ ID NO: 1,

HC1 CBM51 peptide according to SEQ ID NO: 2,

HC7 X408-FN3-CBM5 peptide according to SEQ ID NO:3,

HC11 Bacon-Bacon-CBM32 peptide according to SEQ ID NO: 4, and

HC12 Bacteroides thetaiotaomicron peptide according to SEQ ID NO: 5;

or a mucin-binding targeting agent having 80% sequence identity or more to any one of SEQ ID NO: 1 to 5.

X2. The mucin-binding targeting agent according to item X1 wherein the isolated peptide has a sequence identity of 95% or more to any one of SEQ ID NO:1 to 5.

X3. The mucin-binding targeting agent according to any one of items X1 or X2 wherein the peptide is catalytically inactive against mucins.

X4. The mucin-binding targeting agent according to any one of items X1 to X3 wherein the mucin to which the mucin-targeting agent binds is one or more of MUC2, MUC5AC, MUC5B, and MUC21.

X5. The mucin-binding targeting agent according to any one of items X1 to X4 further comprising a binding moiety.

X6. The mucin-binding targeting agent according to any one of items X1 to X5 further comprising a payload.

X7. The mucin-binding targeting agent according to items X5 or X6 wherein the payload is attached to the agent via the binding moiety.

X8. The mucin-binding targeting agent according to any one of items X5 to X7 wherein the binding moiety is selected from the group comprising esters, lipid anchors, biotin, streptavidin, antibodies, nanobodies, and peptide linkers.

X9. The mucin-binding targeting agent according to any one of items X6 to X8, wherein the payload is selected from the group comprising a therapeutic agent, a detectable marker, nanoparticle, liposome, vesicle and a stain.

X10. The mucin-binding targeting agent according to any of items X1-X9 for use as a medicament.

X11. A composition comprising the mucin-binding targeting agent according to any of items X1-X10.

X12. The composition according to item X11, wherein the composition is a pharmaceutical dosage form further comprising a pharmaceutically acceptable excipient and/or a pharmaceutically acceptable carrier.

X13. The mucin-binding targeting agent according to any of items X1-X10 for use in the treatment of a disease, illness, or disorder in a subject, wherein the disease, illness, or disorder is selected from the group of metabolic, endocrine, inflammatory, immunological diseases, illnesses, or disorders, or is a cancer or a neoplasia.

X14. A method of delivery of a payload to a tissue in a subject, said tissue expressing one or more of MUC2, MUCSAC, MUC5B, and MUC21, said method comprising administering to the subject a pharmaceutical composition comprising a mucin-binding targeting agent comprising an isolated X409 peptide according to SEQ ID NO: 1;

HC1 CBM51 peptide according to SEQ ID NO: 2;

HC7 X408-FN3-CBM5 peptide according to SEQ ID NO:3;

HC11 Bacon-Bacon-CBM32 peptide according to SEQ ID NO: 4; and

HC12 Bacteroides thetaiotaomicron peptide according to SEQ ID NO: 5;

or a mucin-binding targeting agent comprising a sequence having 80% sequence identity to any one of SEQ ID NO: 1 to 5, and a payload bound to said peptide.

X15. A method of preparing a mucin-binding targeting agent, the method comprising the step of providing an isolated

X409 peptide according to SEQ ID NO: 1;

HC1 CBM51 peptide according to SEQ ID NO: 2;

HC7 X408-FN3-CBM5 peptide according to SEQ ID NO:3;

HC11 Bacon-Bacon-CBM32 peptide according to SEQ ID NO: 4; and

HC12 Bacteroides thetaiotaomicron peptide according to SEQ ID NO: 5;

or a mucin-binding targeting agent having 80% sequence identity to any one of SEQ ID NO: 1 to 5.

### Items

1. A mucin-binding targeting agent comprising an isolated X409 peptide according to SEQ ID NO: 1 or a sequence having 65% sequence identity or more thereto.

2. A mucin-binding targeting agent comprising an isolated X409 peptide according to SEQ ID NO: 1 or a sequence having 75% sequence identity or more thereto.

3. A mucin-binding targeting agent comprising an isolated X409 peptide according to SEQ ID NO: 1 or a sequence having 80% sequence identity or more thereto.

4. A mucin-binding targeting agent comprising an isolated X409 peptide according to SEQ ID NO: 1 or a sequence having 85% sequence identity or more thereto.

5. A mucin-binding targeting agent comprising an isolated X409 peptide according to SEQ ID NO: 1 or a sequence having 90% sequence identity or more thereto.

6. A mucin-binding targeting agent comprising an isolated X409 peptide according to SEQ ID NO: 1 or a sequence having 95% sequence identity or more thereto.

7. A mucin-binding targeting agent comprising an isolated X409 peptide according to SEQ ID NO: 1 or a sequence having 96% sequence identity or more thereto.

8. A mucin-binding targeting agent comprising an isolated X409 peptide according to SEQ ID NO: 1 or a sequence having 97% sequence identity or more thereto.

9. A mucin-binding targeting agent comprising an isolated X409 peptide according to SEQ ID NO: 1 or a sequence having 98% sequence identity or more thereto.

10 A mucin-binding targeting agent comprising an isolated X409 peptide according to SEQ ID NO: 1 or a sequence having 99% sequence identity or more thereto.

11. A mucin-binding targeting agent comprising an isolated HC1 CBM51 peptide according to SEQ ID NO: 2 or a sequence having 65% sequence identity or more thereto.

12. A mucin-binding targeting agent comprising an isolated HC1 CBM51 peptide according to SEQ ID NO: 2 or a sequence having 75% sequence identity or more thereto.

13. A mucin-binding targeting agent comprising an isolated HC1 CBM51 peptide according to SEQ ID NO: 2 or a sequence having 80% sequence identity or more thereto.

14. A mucin-binding targeting agent comprising an isolated HC1 CBM51 peptide according to SEQ ID NO: 2 or a sequence having 85% sequence identity or more thereto.

15. A mucin-binding targeting agent comprising an isolated HC1 CBM51 peptide according to SEQ ID NO: 2 or a sequence having 90% sequence identity or more thereto.

16. A mucin-binding targeting agent comprising an isolated HC1 CBM51 peptide according to SEQ ID NO: 2 or a sequence having 95% sequence identity or more thereto.

17. A mucin-binding targeting agent comprising an isolated HC1 CBM51 peptide according to SEQ ID NO: 2 or a sequence having 96% sequence identity or more thereto.

18. A mucin-binding targeting agent comprising an isolated HC1 CBM51 peptide according to SEQ ID NO: 2 or a sequence having 97% sequence identity or more thereto.

19. A mucin-binding targeting agent comprising an isolated HC1 CBM51 peptide according to SEQ ID NO: 2 or a sequence having 98% sequence identity or more thereto.

20 A mucin-binding targeting agent comprising an isolated HC1 CBM51 peptide according to SEQ ID NO: 2 or a sequence having 99% sequence identity or more thereto.

3:
31. A mucin-binding targeting agent comprising an isolated HC7 X408-FN3-CBM5 peptide according to SEQ ID NO: 3 or a sequence having 65% sequence identity or more thereto.

32. A mucin-binding targeting agent comprising an isolated HC7 X408-FN3-CBM5 peptide according to SEQ ID NO: 3 or a sequence having 75% sequence identity or more thereto.

43. A mucin-binding targeting agent comprising an isolated HC7 X408-FN3-CBM5 peptide according to SEQ ID NO: 3 or a sequence having 80% sequence identity or more thereto.

44. A mucin-binding targeting agent comprising an isolated HC7 X408-FN3-CBM5 peptide according to SEQ ID NO: 3 or a sequence having 85% sequence identity or more thereto.

45. A mucin-binding targeting agent comprising an isolated HC7 X408-FN3-CBM5 peptide according to SEQ ID NO: 3 or a sequence having 90% sequence identity or more thereto.

46. A mucin-binding targeting agent comprising an isolated HC7 X408-FN3-CBM5 peptide according to SEQ ID NO: 3 or a sequence having 95% sequence identity or more thereto.

47. A mucin-binding targeting agent comprising an isolated HC7 X408-FN3-CBM5 peptide according to SEQ ID NO: 3 or a sequence having 96% sequence identity or more thereto.

48. A mucin-binding targeting agent comprising an isolated HC7 X408-FN3-CBM5 peptide according to SEQ ID NO: 3 or a sequence having 97% sequence identity or more thereto.

49. A mucin-binding targeting agent comprising an isolated HC7 X408-FN3-CBM5 peptide according to SEQ ID NO: 3 or a sequence having 98% sequence identity or more thereto.

50. A mucin-binding targeting agent comprising an isolated HC7 X408-FN3-CBM5 peptide according to SEQ ID NO: 3 or a sequence having 99% sequence identity or more thereto.

4:

51. A mucin-binding targeting agent comprising an isolated HC11 BACON-BACON-CBM32 peptide according to SEQ ID NO: 4 or a sequence having 65% sequence identity or more thereto.

52. A mucin-binding targeting agent comprising an isolated HC11 BACON-BACON-CBM32 peptide according to SEQ ID NO: 4 or a sequence having 75% sequence identity or more thereto.

53. A mucin-binding targeting agent comprising an isolated HC11 BACON-BACON-CBM32 peptide according to SEQ ID NO: 4 or a sequence having 80% sequence identity or more thereto.

54. A mucin-binding targeting agent comprising an isolated HC11 BACON-BACON-CBM32 peptide according to SEQ ID NO: 4 or a sequence having 85% sequence identity or more thereto.

55. A mucin-binding targeting agent comprising an isolated HC11 BACON-BACON-CBM32 peptide according to SEQ ID NO: 4 or a sequence having 90% sequence identity or more thereto.

56. A mucin-binding targeting agent comprising an isolated HC11 BACON-BACON-CBM32 peptide according to SEQ ID NO: 4 or a sequence having 95% sequence identity or more thereto.

57. A mucin-binding targeting agent comprising an isolated HC11 BACON-BACON-CBM32 peptide according to SEQ ID NO: 4 or a sequence having 96% sequence identity or more thereto.

58. A mucin-binding targeting agent comprising an isolated HC11 BACON-BACON-CBM32 peptide according to SEQ ID NO: 4 or a sequence having 97% sequence identity or more thereto.

59. A mucin-binding targeting agent comprising an isolated HC11 BACON-BACON-CBM32 peptide according to SEQ ID NO: 4 or a sequence having 98% sequence identity or more thereto.

60. A mucin-binding targeting agent comprising an isolated HC11 BACON-BACON-CBM32 peptide according to SEQ ID NO: 4 or a sequence having 99% sequence identity or more thereto.

61. A mucin-binding targeting agent comprising an isolated HC12 BACTEROIDES THETAIOTAOMICRON peptide according to SEQ ID NO: 5 or a sequence having 65% sequence identity or more thereto.

62. A mucin-binding targeting agent comprising an isolated HC12 BACTEROIDES THETAIOTAOMICRON peptide according to SEQ ID NO: 5 or a sequence having 75% sequence identity or more thereto.

63. A mucin-binding targeting agent comprising an isolated HC12 BACTEROIDES THETAIOTAOMICRON peptide according to SEQ ID NO: 5 or a sequence having 80% sequence identity or more thereto.

64. A mucin-binding targeting agent comprising an isolated HC12 BACTEROIDES THETAIOTAOMICRON peptide according to SEQ ID NO: 5 or a sequence having 85% sequence identity or more thereto.

65. A mucin-binding targeting agent comprising an isolated HC12 BACTEROIDES THETAIOTAOMICRON peptide according to SEQ ID NO: 5 or a sequence having 90% sequence identity or more thereto.

66. A mucin-binding targeting agent comprising an isolated HC12 BACTEROIDES THETAIOTAOMICRON peptide according to SEQ ID NO: 5 or a sequence having 95% sequence identity or more thereto.

67. A mucin-binding targeting agent comprising an isolated HC12 BACTEROIDES THETAIOTAOMICRON peptide according to SEQ ID NO: 5 or a sequence having 96% sequence identity or more thereto.

68. A mucin-binding targeting agent comprising an isolated HC12 BACTEROIDES THETAIOTAOMICRON peptide according to SEQ ID NO: 5 or a sequence having 97% sequence identity or more thereto.

69. A mucin-binding targeting agent comprising an isolated HC12 BACTEROIDES THETAIOTAOMICRON peptide according to SEQ ID NO: 5 or a sequence having 98% sequence identity or more thereto.

70 A mucin-binding targeting agent comprising an isolated HC12 BACTEROIDES THETAIOTAOMICRON peptide according to SEQ ID NO: 5 or a sequence having 99% sequence identity or more thereto.

71. The mucin-binding targeting agent according to items 1 to 70 wherein the peptide binds to one or more mucins of mammalian origin.

72. The mucin-binding targeting agent according to any one of items 1 to 71 wherein the peptide binds to one or more mucins of human origin.

73. The mucin-binding targeting agent according to any one of items 1 to 72 wherein the peptide is catalytically inactive against mucins.

74. The mucin-binding targeting agent according to any one of items 1 to 73 wherein the mucin to which the mucin-targeting agent binds is one or more of MUC2, MUC5AC, MUC5B, and MUC21.

75. The mucin-binding targeting agent according to any one of items 1 to 74 further comprising a binding moiety selected from the group comprising a peptide linker, an ester, a lipid anchor, avidin, streptavidin, and biotin.

76. The mucin-binding targeting agent according to item 75 wherein the binding moiety is a lipid anchor.

77. The mucin-binding targeting agent according to item 75 wherein the binding moiety is peptide linker.

78. The mucin-binding targeting agent according to item 75 wherein the binding moiety is an ester.

79. The mucin-binding targeting agent according to any one of items 1 to 78 further comprising a payload.

80. The mucin-binding targeting agent according to item 79 wherein the payload is attached to the agent via the binding moiety.

81. The mucin-binding targeting agent according to any one of items 79 or 80 wherein the payload is covalently attached to the binding moiety.

82. The mucin-binding targeting agent according to any one of items 79 to 81, wherein the payload is selected from the group comprising a therapeutic agent, a detectable marker, nanoparticle, a liposome, a vesicle and a stain.

83. The mucin-binding targeting agent according to item 82, wherein the payload is a therapeutic agent selected from the group comprising radioisotopes, enzymes, antibodies, receptors, RNA, DNA, proteins, therapeutic peptides, and oligonucleotides.

84. The mucin-binding targeting agent according to item 82 wherein the payload is a therapeutic peptide.

85. The mucin-binding targeting agent according to any of the preceding items for use as a medicament.

86. A composition comprising the mucin-binding targeting agent according to any of the preceding items.

87. The composition according to item 86, wherein the composition is a pharmaceutical dosage form further comprising a pharmaceutically acceptable excipient and/or a pharmaceutically acceptable carrier.

88. The composition according to item 87 wherein then composition further comprises a shell and/or an enteric coating.

89. The mucin-binding targeting agent according to any of the preceding items for use in the treatment of a disease, illness, or disorder in a subject, wherein the disease, illness, or disorder is selected from the group of from the group of inflammatory, immunological, endocrine, or metabolic disorders such as obesity or may be neurological, psychological or psychiatric or mood disorders, or disorders of the nervous system, or sexual disorders including reproductive disorders and disorders of the genital system, neoplastic disorders such as cancers, disorders involving dysfunction of mucous tissue or dysfunction of epithelial tissue, including disorders, diseases, and illnesses of the gastrointestinal tract, nasal disorders, disorders and diseases of the eye.

90. The mucin-binding targeting agent according to any of the preceding items, wherein the agent is for oral, rectal, vaginal, buccal, ocular, nasal, or inhalation administration.

91. A method of delivery of a payload to a tissue in a subject, said tissue expressing one or more of MUC2, MUC5AC, MUC5B, and MUC21, said method comprising administering to the subject a pharmaceutical composition comprising a mucin-binding targeting agent comprising an isolated peptide according to any one of SEQ ID NO: 1 to 5 or a sequence having 65% or more, such as 70% or more, such as 80% or more, such as 85% or more, such as 90 percent or more, such as 95% or more, such as 96% or more, such as 97% or more, such as 98% or more, such 99% or more, such as 99.5% or more thereto and a payload bound to said polypeptide

92. The method according to item 91, wherein the tissue is located in the intestinal tract.

93. A method of preparing a mucin-binding targeting agent, the method comprising the step of providing an isolated peptide according to any one of SEQ ID NO: 1 to 5, or a sequence having 65% or more, such as 70% or more, such as 80% or more, such as 85% or more, such as 90 percent or more, such as 95% or more, such as 96% or more, such as 97% or more, such as 98% or more, such 99% or more, such as 99.5% or more thereto.

94. The method according to item 93 further comprising the step of providing a binding moiety and linking the binding moiety and the polypeptide.

95. The method according to item 93 or 94 wherein the peptide and/or the binding moiety is/are produced recombinantly.

96. The method according to any one of items 93 to 95, further comprising a step of attaching a payload to the mucin-binding targeting agent.

97. A mucin-binding targeting agent comprising an isolated mucin-binding peptide sequence that binds an O-glycosylated mucin motif comprised of 5 or more consecutive O-glycans, and which targeting agent do not bind to non-glycosylated mucins independent of the O-glycan structures attached.

98. The mucin-binding targeting agent according to item 97 further comprising a binding moiety.

99. The mucin-binding targeting agent according to item 98 further wherein the binding moiety is selected from the group comprising a peptide linker, an ester, a lipid anchor, avidin, streptavidin, and biotin.

100. The mucin-binding targeting agent according to any one of items 97 to 99 further comprising a payload.

101. The mucin-binding targeting agent according to item 100 wherein the payload is selected from the group comprising a therapeutic agent, a detectable marker, nanoparticle, a liposome, a vesicle and a stain.

102. The mucin-binding targeting agent according to item 101 wherein the payload is a therapeutic agent.

103. A DNA sequence encoding any one of the peptides according to SEQ ID NO: 1 to 5 or a sequence having 65% or more, such as 70% or more, such as 80% or more, such as 85% or more, such as 90 percent or more, such as 95% or more, such as 96% or more, such as 97% or more, such as 98% or more, such 99% or more, such as 99.5% or more sequence identity thereto.

1. A mucin-binding targeting agent comprising an isolated peptide comprising
i) a X409 peptide according to SEQ ID NO: 1, or an isolated peptide having 75% sequence identity or more to SEQ ID NO: 1, and
ii) a payload.

2. The mucin-binding targeting agent according to claim 1, wherein the isolated peptide is catalytically inactive against mucins.

3. The mucin-binding targeting agent according to claim 1, wherein the mucin to which the mucin-binding targeting agent binds is one or more of MUC2, MUC5AC, MUC5B, and MUC21.

4. The mucin-binding targeting agent according to claim 1, wherein the binding of the mucin-binding targeting agent to MUC5AC has a dissociation constant of less than 1 μM, such as less than 500 nM, such as less than 250 nM, such as less than 100 nM, preferably less than 50 nM.

5. The mucin-binding targeting agent according to claim 1, wherein the binding of the mucin-binding targeting agent to MUC1 has a dissociation constant of more than 1 μM.

6. The mucin-binding targeting agent according to claim 1, wherein each mucin-binding targeting agent molecule binds only a single mucin molecule.

7. The mucin-binding targeting agent according to claim 1, wherein the isolated peptide is a X409 peptide selected from the group consisting of SEQ ID NO: 1, SEQ ID NO:73, SEQ ID NO: 74, and SEQ ID NO: 135.

8. The mucin-binding targeting agent according to claim 1 further comprising a binding moiety.

9. The mucin-binding targeting agent according to claim 8, wherein the payload is attached to the mucin-binding targeting agent via the binding moiety.

10. The mucin-binding targeting agent according to claim 8, wherein the binding moiety is selected from the group consisting of esters, lipid anchors, biotin, streptavidin, antibodies, nanobodies, and peptide linkers.

11. The mucin-binding targeting agent according to claim 1, wherein the payload is selected from the group consisting of a therapeutic agent, an enzyme, a vaccine, a peptide hormone, a small molecule drug, a detectable marker, nanoparticle, liposome, vesicle and a stain.

12. The mucin-binding targeting agent according to claim 11, wherein the enzyme is selected from the group consisting of proteases, lipases, phytases, amylase, xylanases, β-Glucanases, α-Galactosidases, mannanases, cellulases, hemicellulases, and pectinases.

13. A composition comprising the mucin-binding targeting agent according to claim 1.

14. The composition according to claim 13, wherein the composition is a pharmaceutical dosage form further comprising a pharmaceutically acceptable excipient and/or a pharmaceutically acceptable carrier.

15. The mucin-binding targeting agent according to claim 1 for use as a medicament.

16. The mucin-binding targeting agent according to claim 15 for use in the treatment of a disease, illness, or disorder in a subject, wherein the disease, illness, or disorder is selected from the group of metabolic, endocrine, inflammatory, immunological diseases, illnesses, or disorders, or is a cancer or a neoplasia.

17-103. (canceled)
